SEQUENCE LISTING (13 sequences)

SEQ ID NO: 1 - protein, 12 aa, Artificial Sequence
  Synthetic peptide
TSFAEYWNLLSP

SEQ ID NO: 2 - DNA, 1446 bp, Stevia rebaudiana
atggatgcaatggcaactactgagaaaaagcctcatgtgatcttcattccatttcctgcacaatctcacataaaggcaatgctaaagttagcacaactattacaccataagggattacagataactttcgtgaataccgacttcatccataatcaatttctggaatctagtggccctcattgtttggacggagccccagggtttagattcgaaacaattcctgacggtgtttcacattccccagaggcctccatcccaataagagagagtttactgaggtcaatagaaaccaactttttggatcgtttcattgacttggtcacaaaacttccagacccaccaacttgcataatctctgatggctttctgtcagtgtttactatcgacgctgccaaaaagttgggtatcccagttatgatgtactggactcttgctgcatgcggtttcatgggtttctatcacatccattctcttatcgaaaagggttttgctccactgaaagatgcatcatacttaaccaacggctacctggatactgttattgactgggtaccaggtatggaaggtataagacttaaagattttcctttggattggtctacagaccttaatgataaagtattgatgtttactacagaagctccacaaagatctcataaggtttcacatcatatctttcacacctttgatgaattggaaccatcaatcatcaaaaccttgtctctaagatacaatcatatctacactattggtccattacaattacttctagatcaaattcctgaagagaaaaagcaaactggtattacatccttacacggctactctttagtgaaagaggaaccagaatgttttcaatggctacaaagtaaagagcctaattctgtggtctacgtcaacttcggaagtacaacagtcatgtccttggaagatatgactgaatttggttggggccttgctaattcaaatcattactttctatggattatcaggtccaatttggtaataggggaaaacgccgtattacctccagaattggaggaacacatcaaaaagagaggtttcattgcttcctggtgttctcaggaaaaggtattgaaacatccttctgttggtggtttccttactcattgcggttggggctctacaatcgaatcactaagtgcaggagttccaatgatttgttggccatattcatgggaccaacttacaaattgtaggtatatctgtaaagagtgggaagttggattagaaatgggaacaaaggttaaacgtgatgaagtgaaaagattggttcaggagttgatgggggaaggtggccacaagatgagaaacaaggccaaagattggaaggaaaaagccagaattgctattgctcctaacgggtcatcctctctaaacattgataagatggtcaaagagattacagtcttagccagaaactaa

SEQ ID NO: 3 - protein, 660 aa, Stevia rebaudiana
MQSNSVKISPLDLVTALFSGKVLDTSNASESGESAMLPTIAMIMENRELLMILTTSVAVLIGCVVVLVWRRSSTKKSALEPPVIVVPKRVQEEEVDDGKKKVTVFFGTQTGTAEGFAKALVEEAKARYEKAVFKVIDLDDYAADDDEYEEKLKKESLAFFFLATYGDGEPTDNAARFYKWFTEGDAKGEWLNKLQYGVFGLGNRQYEHFNKIAKVVDDGLVEQGAKRLVPVGLGDDDQCIEDDFTAWKELVWPELDQLLRDEDDTTVATPYTAAVAEYRVVFHEKPDALSEDYSYTNGHAVHDAQHPCRSNVAVKKELHSPESDRSCTHLEFDISNTGLSYETGDHVGVYCENLSEVVNDAERLVGLPPDTYSSIHTDSEDGSPLGGASLPPPFPPCTLRKALTCYADVLSSPKKSALLALAAHATDPSEADRLKFLASPAGKDEYSQWIVASQRSLLEVMEAFPSAKPSLGVFFASVAPRLQPRYYSISSSPKMAPDRIHVTCALVYEKTPAGRIHKGVCSTWMKNAVPMTESQDCSWAPIYVRTSNFRLPSDPKVPVIMIGPGTGLAPFRGFLQERLALKEAGTDLGLSILFFGCRNRKVDFIYENELNNFVETGALSELIVAFSREGPTKEYVQHKMSEKASDIWNLLSEGAYLYVC

SEQ ID NO: 4 - DNA, 2124 bp, Stevia rebaudiana
atgcaatctaactccgtgaagatttcgccgcttgatctggtaactgcgctgtttagcggcaaggttttggacacatcgaacgcatcggaatcgggagaatctgctatgctgccgactatagcgatgattatggagaatcgtgagctgttgatgatactcacaacgtcggttgctgtattgatcggatgcgttgtcgttttggtgtggcggagatcgtctacgaagaagtcggcgttggagccaccggtgattgtggttccgaagagagtgcaagaggaggaagttgatgatggtaagaagaaagttacggttttcttcggcacccaaactggaacagctgaaggcttcgctaaggcacttgttgaggaagctaaagctcgatatgaaaaggctgtctttaaagtaattgatttggatgattatgctgctgatgacgatgagtatgaggagaaactaaagaaagaatctttggcctttttctttttggctacgtatggagatggtgagccaacagataatgctgccagattttataaatggtttactgagggagatgcgaaaggagaatggcttaataagcttcaatatggagtatttggtttgggtaacagacaatatgaacattttaacaagatcgcaaaagtggttgatgatggtcttgtagaacagggtgcaaagcgtcttgttcctgttggacttggagatgatgatcaatgtattgaagatgacttcaccgcatggaaagagttagtatggccggagttggatcaattacttcgtgatgaggatgacacaactgttgctactccatacacagctgctgttgcagaatatcgcgttgtttttcatgaaaaaccagacgcgctttctgaagattatagttatacaaatggccatgctgttcatgatgctcaacatccatgcagatccaacgtggctgtcaaaaaggaacttcatagtcctgaatctgaccggtcttgcactcatcttgaatttgacatctcgaacaccggactatcatatgaaactggggaccatgttggagtttactgtgaaaacttgagtgaagttgtgaatgatgctgaaagattagtaggattaccaccagacacttactcctccatccacactgatagtgaagacgggtcgccacttggcggagcctcattgccgcctcctttcccgccatgcactttaaggaaagcattgacgtgttatgctgatgttttgagttctcccaagaagtcggctttgcttgcactagctgctcatgccaccgatcccagtgaagctgatagattgaaatttcttgcatcccccgccggaaaggatgaatattctcaatggatagttgcaagccaaagaagtctccttgaagtcatggaagcattcccgtcagctaagccttcacttggtgttttctttgcatctgttgccccgcgcttacaaccaagatactactctatttcttcctcacccaagatggcaccggataggattcatgttacatgtgcattagtctatgagaaaacacctgcaggccgcatccacaaaggagtttgttcaacttggatgaagaacgcagtgcctatgaccgagagtcaagattgcagttgggccccaatatacgtccgaacatccaatttcagactaccatctgaccctaaggtcccggttatcatgattggacctggcactggtttggctccttttagaggtttccttcaagagcggttagctttaaaggaagccggaactgacctcggtttatccattttattcttcggatgtaggaatcgcaaagtggatttcatatatgaaaacgagcttaacaactttgtggagactggtgctctttctgagcttattgttgctttctcccgtgaaggcccgactaaggaatatgtgcaacacaagatgagtgagaaggcttcggatatctggaacttgctttctgaaggagcatatttatacgtatgtggtgatgccaaaggcatggccaaagatgtacatcgaaccctccacacaattgtgcaagaacagggatctcttgactcgtcaaaggcagaactctacgtgaagaatctacaaatgtcaggaagatacctccgtgacgtttggtaa

SEQ ID NO: 5 - DNA, 2506 bp, Arabidopsis thaliana
cgtcagtcatcaaggctaattcgtcgcgagttgctacgacgccgtttcggttgcttctggtttctttatgtctatcaaccttcgctcctccggttgttcgtctccgatctcagctactttggaacgaggattggactcagaagtacagacaagagctaacaatgtgagctttgagcaaacaaaggagaagattaggaagatgttggagaaagtggagctttctgtttcggcctacgatactagttgggtagcaatggttccatcaccgagctcccaaaatgctccacttttcccacagtgtgtgaaatggttattggataatcaacatgaagatggatcttggggacttgataaccatgaccatcaatctcttaagaaggatgtgttatcatctacactggctagtatcctcgcgttaaagaagtggggaattggtgaaagacaaataaacaagggtctccagtttattgagctgaattctgcattagtcactgatgaaaccatacagaaaccaacagggtttgatattatatttcctgggatgattaaatatgctagagatttgaatctgacgattccattgggctcagaagtggtggatgacatgatacgaaaaagagatctggatcttaaatgtgatagtgaaaagttttcaaagggaagagaagcatatctggcctatgttttagaggggacaagaaacctaaaagattgggatttgatagtcaaatatcaaaggaaaaatgggtcactgtttgattctccagccacaacagcagctgcttttactcagtttgggaatgatggttgtctccgttatctctgttctctccttcagaaattcgaggctgcagttccttcagtttatccatttgatcaatatgcacgccttagtataattgtcactcttgaaagcttaggaattgatagagatttcaaaaccgaaatcaaaagcatattggatgaaacctatagatattggcttcgtggggatgaagaaatatgtttggacttggccacttgtgctttggctttccgattattgcttgctcatggctatgatgtgtcttacgatccgctaaaaccatttgcagaagaatctggtttctctgatactttggaaggatatgttaagaatacgttttctgtgttagaattatttaaggctgctcaaagttatccacatgaatcagctttgaagaagcagtgttgttggactaaacaatatctggagatggaattgtccagctgggttaagacctctgttcgagataaatacctcaagaaagaggtcgaggatgctcttgcttttccctcctatgcaagcctagaaagatcagatcacaggagaaaaatactcaatggttctgctgtggaaaacaccagagttacaaaaacctcatatcgtttgcacaatatttgcacctctgatatcctgaagttagctgtggatgacttcaatttctgccagtccatacaccgtgaagaaatggaacgtcttgataggtggattgtggagaatagattgcaggaactgaaatttgccagacagaagctggcttactgttatttctctggggctgcaactttattttctccagaactatctgatgctcgtatatcgtgggccaaaggtggagtacttacaacggttgtagacgacttctttgatgttggagggtccaaagaagaactggaaaacctcatacacttggtcgaaaagtgggatttgaacggtgttcctgagtacagctcagaacatgttgagatcatattctcagttctaagggacaccattctcgaaacaggagacaaagcattcacctatcaaggacgcaatgtgacacaccacattgtgaaaatttggttggatctgctcaagtctatgttgagagaagccgagtggtccagtgacaagtcaacaccaagcttggaggattacatggaaaatgcgtacatatcatttgcattaggaccaattgtcctcccagctacctatctgatcggacctccacttccagagaagacagtcgatagccaccaatataatcagctctacaagctcgtgagcactatgggtcgtcttctaaatgacatacaaggttttaagagagaaagcgcggaagggaagctgaatgcggtttcattgcacatgaaacacgagagagacaatcgcagcaaagaagtgatcatagaatcgatgaaaggtttagcagagagaaagagggaagaattgcataagctagttttggaggagaaaggaagtgtggttccaagggaatgcaaagaagcgttcttgaaaatgagcaaagtgttgaacttattttacaggaaggacgatggattcacatcaaatgatctgatgagtcttgttaaatcagtgatctacgagcctgttagcttacagaaagaatctttaacttgatccaagttgatctggcaggtaaactcagtaaatgaaaataagactttggtcttcttctttgttgcttcagaacaagaagag

SEQ ID NO: 6 - DNA, 1503 bp, Stevia rebaudiana
atggaagcctcttacctatacatttctattttgcttttactggcatcatacctgttcaccactcaacttagaaggaagagcgctaatctaccaccaaccgtgtttccatcaataccaatcattggacacttatacttactcaaaaagcctctttatagaactttagcaaaaattgccgctaagtacggaccaatactgcaattacaactcggctacagacgtgttctggtgatttcctcaccatcagcagcagaagagtgctttaccaataacgatgtaatcttcgcaaatagacctaagacattgtttggcaaaatagtgggtggaacatcccttggcagtttatcctacggcgatcaatggcgtaatctaaggagagtagcttctatcgaaatcctatcagttcataggttgaacgaatttcatgatatcagagtggatgagaacagattgttaattagaaaacttagaagttcatcttctcctgttactcttataacagtcttttatgctctaacattgaacgtcattatgagaatgatctctggcaaaagatatttcgacagtggggatagagaattggaggaggaaggtaagagatttcgagaaatcttagacgaaacgttgcttctagccggtgcttctaatgttggcgactacttaccaatattgaactggttgggagttaagtctcttgaaaagaaattgatcgctttgcagaaaaagagagatgactttttccagggtttgattgaacaggttagaaaatctcgtggtgctaaagtaggcaaaggtagaaaaacgatgatcgaactcttattatctttgcaagagtcagaacctgagtactatacagatgctatgataagatcttttgtcctaggtctgctggctgcaggtagtgatacttcagcgggcactatggaatgggccatgagcttactggtcaatcacccacatgtattgaagaaagctcaagctgaaatcgatagagttatcggtaataacagattgattgacgagtcagacattggaaatatcccttacatcgggtgtattatcaatgaaactctaagactctatccagcagggccattgttgttcccacatgaaagttctgccgactgcgttatttccggttacaatatacctagaggtacaatgttaatcgtaaaccaatgggcgattcatcacgatcctaaagtctgggatgatcctgaaacctttaaacctgaaagatttcaaggattagaaggaactagagatggtttcaaacttatgccattcggttctgggagaagaggatgtccaggtgaaggtttggcaataaggctgttagggatgacactaggctcagtgatccaatgttttgattgggagagagtaggagatgagatggttgacatgacagaaggtttgggtgtcacacttcctaaggccgttccattagttgccaaatgtaagccacgttccgaaatgactaatctcctatccgaactttaa

SEQ ID NO: 7 - protein, 500 aa, Stevia rebaudiana
MEASYLYISILLLLASYLFTTQLRRKSANLPPTVFPSIPIIGHLYLLKKPLYRTLAKIAAKYGPILQLQLGYRRVLVISSPSAAEECFTNNDVIFANRPKTLFGKIVGGTSLGSLSYGDQWRNLRRVASIEILSVHRLNEFHDIRVDENRLLIRKLRSSSSPVTLITVFYALTLNVIMRMISGKRYFDSGDRELEEEGKRFREILDETLLLAGASNVGDYLPILNWLGVKSLEKKLIALQKKRDDFFQGLIEQVRKSRGAKVGKGRKTMIELLLSLQESEPEYYTDAMIRSFVLGLLAAGSDTSAGTMEWAMSLLVNHPHVLKKAQAEIDRVIGNNRLIDESDIGNIPYIGCIINETLRLYPAGPLLFPHESSADCVISGYNIPRGTMLIVNQWAIHHDPKVWDDPETFKPERFQGLEGTRDGFKLMPFGSGRRGCPGEGLAIRLLGMTLGSVIQCFDWERVGDEMVDMTEGLGVTLPKAVPLVAKCKPRSEMTNLLSEL

SEQ ID NO: 8 - DNA, 2489 bp, Zea mays
atggttttgtcttcttcttgtactacagtaccacacttatcttcattagctgtcgtgcaacttggtccttggagcagtaggattaaaaagaaaaccgatactgttgcagtaccagccgctgcaggaaggtggagaagggccttggctagagcacagcacacatcagaatccgcagctgtcgcaaagggcagcagtttgacccctatagtgagaactgacgctgagtcaaggagaacaagatggccaaccgatgacgatgacgccgaacctttagtggatgagatcagggcaatgcttacttccatgtctgatggtgacatttccgtgagcgcatacgatacagcctgggtcggattggttccaagattagacggcggtgaaggtcctcaatttccagcagctgtgagatggataagaaataaccagttgcctgacggaagttggggcgatgccgcattattctctgcctatgacaggcttatcaatacccttgcctgcgttgtaactttgacaaggtggtccctagaaccagagatgagaggtagaggactatcttttttgggtaggaacatgtggaaattagcaactgaagatgaagagtcaatgcctattggcttcgaattagcatttccatctttgatagagcttgctaagagcctaggtgtccatgacttcccttatgatcaccaggccctacaaggaatctactcttcaagagagatcaaaatgaagaggattccaaaagaagtgatgcataccgttccaacatcaatattgcacagtttggagggtatgcctggcctagattgggctaaactacttaaactacagagcagcgacggaagttttttgttctcaccagctgccactgcatatgctttaatgaataccggagatgacaggtgttttagctacatcgatagaacagtaaagaaattcaacggcggcgtccctaatgtttatccagtggatctatttgaacatatttgggccgttgatagacttgaaagattaggaatctccaggtacttccaaaaggagatcgaacaatgcatggattatgtaaacaggcattggactgaggacggtatttgttgggcaaggaactctgatgtcaaagaggtggacgacacagctatggcctttagacttcttaggttgcacggctacagcgtcagtcctgatgtgtttaaaaacttcgaaaagacggtgaatttttcgcatttgtcggacagtctaatcaagctgttaccggtatgtacaacttaaacagagcaagccagatatccttcccaggcgaggatgtgcttcatagagctggtgccttctcatatgagttcttgaggagaaaagaagcagagggagctttgagggacaagtggatcatttctaaagatctacctggtgaagttgtgtatactttggattttccatggtacggcaacttacctagagtcgaggccagagactacctagagcaatacggaggtggtgatgacgtttggattggcaagacattgtataggatgccacttgtaaacaatgatgtatatttggaattggcaagaatggatttcaaccactgccaggctttgcatcagttagagtggcaaggactaaaaagatggtatactgaaaataggttgatggactttggtgtcgcccaagaagatgcccttagagcttattttcttgcagccgcatctgtttacgagccttgtagagctgccgagaggcttgcatgggctagagccgcaatactagctaacgccgtgagcacccacttaagaaatagcccatcattcagagaaaggttagagcattctcttaggtgtagacctagtgaagagacagatggctcctggtttaactcctcaagtggctctgatgcagttttagtaaaggctgtcttaagacttactgattcattagccagggaagcacagccaatccatggaggtgacccagaagatattatacacaagttgttaagatctgcttgggccgagtgggttagggaaaaggcagacgctgccgatagcgtgtgcaatggtagttctgcagtagaacaagagggatcaagaatggtccatgataaacagacctgtctattattggctagaatgatcgaaatttctgccggtagggcagctggtgaagcagccagtgaggacggcgatagaagaataattcaattaacaggctccatctgcgacagtcttaagcaaaaaatgctagtttcacaggaccctgaaaaaaatgaagagatgatgtctcacgtggatgacgaattgaagttgaggattagagagttcgttcaatatttgcttagactaggtgaaaaaaagactggatctagcgaaaccaggcaaacatttttaagtatagtgaaatcatgttactatgctgctcattgcccacctcatgtcgttgatagacacattagtagagtgattttcgagccagtaagtgccgcaaagtaaccgcgg

SEQ ID NO: 9 - protein, 827 aa, Zea mays
MVLSSSCTTVPHLSSLAVVQLGPWSSRIKKKTDTVAVPAAAGRWRRALARAQHTSESAAVAKGSSLTPIVRTDAESRRTRWPTDDDDAEPLVDEIRAMLTSMSDGDISVSAYDTAWVGLVPRLDGGEGPQFPAAVRWIRNNQLPDGSWGDAALFSAYDRLINTLACVVTLTRWSLEPEMRGRGLSFLGRNMWKLATEDEESMPIGFELAFPSLIELAKSLGVHDFPYDHQALQGIYSSREIKMKRIPKEVMHTVPTSILHSLEGMPGLDWAKLLKLQSSDGSFLFSPAATAYALMNTGDDRCFSYIDRTVKKFNGGVPNVYPVDLFEHIWAVDRLERLGISRYFQKEIEQCMDYVNRHWTEDGICWARNSDVKEVDDTAMAFRLLRLHGYSVSPDVFKNFEKDGEFFAFVGQSNQAVTGMYNLNRASQISFPGEDVLHRAGAFSYEFLRRKEAEGALRDKWIISKDLPGEVVYTLDFPWYGNLPRVEARDYLEQYGGGDDVWIGKTLYRMPLVNNDVYLELARMDFNHCQALHQLEWQGLKRWYTENRLMDFGVAQEDALRAYFLAAASVYEPCRAAERLAWARAAILANAVSTHLRNSPSFRERLEHSLRCRPSEETDGSWFNSSSGSDAVLVKAVLRLTDSLAREAQPIHGGDPEDIIHKLLRSAWAEWVREKADAADSVCNGSSAVEQEGSRMVHDKQTCLLLARMIEISAGRAAGEAASEDGDRRIIQLTGSICDSLKQKMLVSQDPEKNEEMMSHVDDELKLRIREFVQYLLRLGEKKTGSSETRQTFLSIVKSCYYAAHCPPHVVDRHISRVIFEPVSAAK

SEQ ID NO: 10 - protein, 712 aa, Arabidopsis thaliana
MSSSSSSSTSMIDLMAAIIKGEPVIVSDPANASAYESVAAELSSMLIENRQFAMIVTTSIAVLIGCIVMLVWRRSGSGNSKRVEPLKPLVIKPREEEIDDGRKKVTIFFGTQTGTAEGFAKALGEEAKARYEKTRFKIVDLDDYAADDDEYEEKLKKEDVAFFFLATYGDGEPTDNAARFYKWFTEGNDRGEWLKNLKYGVFGLGNRQYEHFNKVAKVVDDILVEQGAQRLVQVGLGDDDQCIEDDFTAWREALWPELDTILREEGDTAVATPYTAAVLEYRVSIHDSEDAKFNDITLANGNGYTVFDAQHPYKANVAVKRELHTPESDRSCIHLEFDIAGSGLTMKLGDHVGVLCDNLSETVDEALRLLDMSPDTYFSLHAEKEDGTPISSSLPPPFPPCNLRTALTRYACLLSSPKKSALVALAAHASDPTEAERLKHLASPAGKDEYSKWVVESQRSLLEVMAEFPSAKPPLGVFFAGVAPRLQPRFYSISSSPKIAETRIHVTCALVYEKMPTGRIHKGVCSTWMKNAVPYEKSEKLFLGRPIFVRQSNFKLPSDSKVPIIMIGPGTGLAPFRGFLQERLALVESGVELGPSVLFFGCRNRRMDFIYEEELQRFVESGALAELSVAFSREGPTKEYVQHKMMDKASDIWNMISQGAYLYVCGDAKGMARDVHRSLHTIAQEQGSMDSTKAEGFVKNLQTSGRYLRDVW

SEQ ID NO: 11 - DNA, 2139 bp, Arabidopsis thaliana
atgtcttcctcttcctcttccagtacctctatgattgatttgatggctgctattattaaaggtgaaccagttatcgtctccgacccagcaaatgcctctgcttatgaatcagttgctgcagaattgtcttcaatgttgatcgaaaacagacaattcgccatgatcgtaactacatcaatcgctgttttgatcggttgtattgtcatgttggtatggagaagatccggtagtggtaattctaaaagagtcgaacctttgaaaccattagtaattaagccaagagaagaagaaatagatgacggtagaaagaaagttacaatatttttcggtacccaaactggtacagctgaaggttttgcaaaagccttaggtgaagaagctaaggcaagatacgaaaagactagattcaagatagtcgatttggatgactatgccgctgatgacgatgaatacgaagaaaagttgaagaaagaagatgttgcatttttctttttggcaacctatggtgacggtgaaccaactgacaatgcagccagattctacaaatggtttacagagggtaatgatcgtggtgaatggttgaaaaacttaaagtacggtgttttcggtttgggtaacagacaatacgaacatttcaacaaagttgcaaaggttgtcgacgatattttggtcgaacaaggtgctcaaagattagtccaagtaggtttgggtgacgatgaccaatgtatagaagatgactttactgcctggagagaagctttgtggcctgaattagacacaatcttgagagaagaaggtgacaccgccgttgctaccccatatactgctgcagtattagaatacagagtttccatccatgatagtgaagacgcaaagtttaatgatatcactttggccaatggtaacggttatacagttttcgatgcacaacacccttacaaagctaacgttgcagtcaagagagaattacatacaccagaatccgacagaagttgtatacacttggaatttgatatcgctggttccggtttaaccatgaagttgggtgaccatgtaggtgttttatgcgacaatttgtctgaaactgttgatgaagcattgagattgttggatatgtcccctgacacttattttagtttgcacgctgaaaaagaagatggtacaccaatttccagttctttaccacctccattccctccatgtaacttaagaacagccttgaccagatacgcttgcttgttatcatcccctaaaaagtccgccttggttgctttagccgctcatgctagtgatcctactgaagcagaaagattgaaacacttagcatctccagccggtaaagatgaatattcaaagtgggtagttgaatctcaaagatcattgttagaagttatggcagaatttccatctgccaagcctccattaggtgtcttctttgctggtgtagcacctagattgcaaccaagattctactcaatcagttcttcacctaagatcgctgaaactagaattcatgttacatgtgcattagtctacgaaaagatgccaaccggtagaattcacaagggtgtatgctctacttggatgaaaaatgctgttccttacgaaaaatcagaaaagttgttcttaggtagaccaatcttcgtaagacaatcaaacttcaagttgccttctgattcaaaggttccaataatcatgataggtcctggtacaggtttagccccattcagaggtttcttgcaagaaagattggctttagttgaatctggtgtcgaattaggtccttcagttttgttctttggttgtagaaacagaagaatggatttcatctatgaagaagaattgcaaagattcgtcgaatctggtgcattggccgaattatctgtagctttttcaagagaaggtccaactaaggaatacgttcaacataagatgatggataaggcatccgacatatggaacatgatcagtcaaggtgcttatttgtacgtttgcggtgacgcaaagggtatggccagagatgtccatagatctttgcacacaattgctcaagaacaaggttccatggatagtaccaaagctgaaggtttcgtaaagaacttacaaacttccggtagatacttgagagatgtctggtga

SEQ ID NO: 12 - DNA, 1377 bp, Stevia rebaudiana
atggaaaacaagaccgaaacaacagttagacgtaggcgtagaatcattctgtttccagtaccttttcaagggcacatcaatccaatactacaactagccaacgttttgtactctaaaggtttttctattacaatctttcacaccaatttcaacaaaccaaaaacatccaattacccacatttcacattcagattcatacttgataatgatccacaagatgaacgtatttcaaacttacctacccacggtcctttagctggaatgagaattccaatcatcaatgaacatggtgccgatgagcttagaagagaattagagttacttatgttggcatccgaagaggacgaggaagtctcttgtctgattactgacgctctatggtactttgcccaatctgtggctgatagtttgaatttgaggagattggtactaatgacatccagtctgtttaactttcacgctcatgttagtttaccacaatttgacgaattgggatacttggaccctgatgacaagactaggttagaggaacaggcctctggttttcctatgttgaaagtcaaagatatcaagtctgcctattctaattggcaaatcttgaaagagatcttaggaaagatgatcaaacagacaaaggcttcatctggagtgatttggaacagtttcaaagagttagaagagtctgaattggagactgtaatcagagaaattccagcaccttcattcctgataccattaccaaaacatttgactgcttcctcttcctctttgttggatcatgacagaacagtttttcaatggttggaccaacaaccacctagttctgttttgtacgtgtcatttggtagtacttctgaagtcgatgaaaaggacttccttgaaatcgcaagaggcttagtcgatagtaagcagtcattcctttgggtcgtgcgtccaggtttcgtgaaaggctcaacatgggtcgaaccacttccagatggttttctaggcgaaagaggtagaatagtcaaatgggttcctcaacaggaagttttagctcatggcgctattggggcattctggactcattccggatggaattcaactttagaatcagtatgcgaaggggtacctatgatcttttcagattttggtcttgatcaaccactgaacgcaagatacatgtctgatgttttgaaagtgggtgtatatctagaaaatggctgggaaaggggtgaaatagctaatgcaataagacgtgttatggttgatgaagagggggagtatatcagacaaaacgcaagagtgctgaagcaaaaggccgacgtttctctaatgaagggaggctcttcatacgaatccttagaatctcttgtttcctacatttcatcactgtaa

SEQ ID NO: 13 - protein, 473 aa, Stevia rebaudiana
MATSDSIVDDRKQLHVATFPWLAFGHILPYLQLSKLIAEKGHKVSFLSTTRNIQRLSSHISPLINVVQLTLPRVQELPEDAEATTDVHPEDIPYLKKASDGLQPEVTRFLEQHSPDWIIYDYTHYWLPSIAASLGISRAHFSVTTPWAIAYMGPSADAMINGSDGRTTVEDLTTPPKWFPFPTKVCWRKHDLARLVPYKAPGISDGYRMGLVLKGSDCLLSKCYHEFGTQWLPLLETLHQVPVVPVGLLPPEIPGDEKDETWVSIKKWLDGKQKGSVVYVALGSEVLVSQTEVVELALGLELSGLPFVWAYRKPKGPAKSDSVELPDGFVERTRDRGLVWTSWAPQLRILSHESVCGFLTHCGSGSIVEGLMFGHPLIMLPIFGDQPLNARLLEDKQVGIEIPRNEEDGCLTKESVARSLRSVVVEKEGEIYKANARELSKIYNDTKVEKEYVSQFVDYLEKNARAVAIDHES

(19) United States
(12) Patent Application Publication
(10) Pub. No.: US 2015/0342234 A1
(43) Pub. Date: Dec. 3, 2015

(54) Steviol Glycoside Compositions Sensory Properties

(71) Applicant: Evolva SA, Reinach (CH)

(72) Inventors: Paula Hicks, Bend, OR (US); Michael Mikkelsen, Vaerlose (DK); Jens Houghton-Larsen, Birkerod (DK); Jorgen Hansen, Frederiksberg (DK); Hans Peter Smits, Holte (DK); Jens Mueller, Berlin (DE)

(21) Appl. No.: 14/648,747

(22) PCT Filed: Dec. 4, 2013
(86) PCT No.: PCT/EP2013/075587

§ 371 Date: Jun. 1, 2015

Related U.S. Application Data
(60) Provisional application No. 61/733,693, filed on Dec. 5, 2012.

(51) Int. Cl. A23L 1/236; C12P 19/56

(52) U.S. Cl. CPC A23L 1/2363; C12P 19/56; A23V 2002/00

(57) ABSTRACT

Materials and methods for producing particular steviol glycosides, and high-purity compositions of particular steviol glycosides with improved sensory profilesare provided herein. The steviol glycosides can be, for example, rebaudioside D, rebaudioside A, and rebaudioside B, and can be produced by recombinant microorganisms.

### TECHNICAL FIELD

This disclosure relates to the recombinant production of steviol glycosides and isolation methods thereof. In particular, this disclosure relates to the production of steviol glycosides such as rebaudioside D, rebaudioside A, and rebaudioside B by recombinant hosts such as recombinant microorganisms, as well as methods for isolation or enrichment of particular steviol glycosides, and high-purity compositions of particular steviol glycosides with improved sensory profiles.

### BACKGROUND

Sweeteners are well known as ingredients used most commonly in the food, beverage, or confectionary industries. The sweetener can either be incorporated into a final food product during production or for stand-alone use, when appropriately diluted, as a tabletop sweetener or an at-home replacement for sugars in baking. Sweeteners include natural sweeteners such as sucrose, high fructose corn syrup, molasses, maple syrup, and honey and artificial sweeteners such as aspartame, saccharine and sucralose. Stevia extract is a natural sweetener that can be isolated and extracted from a perennial shrub, Stevia rebaudiana. Stevia is commonly grown in South America and Asia for commercial production of stevia extract. Stevia extract, purified to various degrees, is used commercially as a high intensity sweetener in foods and in blends or alone as a tabletop sweetener.

Extracts of the Stevia plant contain rebaudiosides and other steviol glycosides that contribute to the sweet flavor, although the amount of each glycoside often varies among different production batches. Existing commercial products are predominantly rebaudioside A with lesser amounts of other glycosides such as rebaudioside C, D, and F. Stevia extracts may also contain contaminants such as plant-derived compounds that contribute to off-flavors. These off-flavors can be more or less problematic depending on the food system or application of choice. Potential contaminants include pigments, lipids, proteins, phenolics, saccharides, spathulenol and other sesquiterpenes, labdane diterpenes, monoterpenes, decanoic acid, 8,11,14-eicosatrienoic acid, 2-methyloctadecane, pentacosane, octacosane, tetracosane, octadecanol, stigmasterol, β-sitosterol, α- and β-amyrin, lupeol, β-amryin acetate, pentacyclic triterpenes, centauredin, quercitin, epi-alpha-cadinol, carophyllenes and derivatives, beta-pinene, beta-sitosterol, and gibberellin.

### SUMMARY

Typically, stevioside and rebaudioside A are the primary compounds in commercially-produced stevia extracts. Stevioside is reported to have a more bitter and less sweet taste than rebaudioside A. The composition of stevia extract can vary from lot to lot depending on the soil and climate in which the plants are grown. Depending upon the sourced plant, the climate conditions, and the extraction process, the amount of rebaudioside A in commercial preparations is reported to vary from 20 to 97% of the total steviol glycoside content. Other steviol glycosides are present in varying amounts in stevia extracts. For example, rebaudioside B is typically present at less than 1-2%, whereas rebaudioside C can be present at levels as high as 7-15%. Rebaudioside D is typically present in levels of 2% or less, and rebaudioside F is typically present in compositions at 3.5% or less of the total steviol glycosides. The amount of the minor steviol glycosides affects the flavor profile of a Stevia extract. The materials and methods described herein can be useful for producing steviol glycoside compositions having increased amounts of one or more compounds (e.g., rebaudioside A) for use, for example, as non-caloric sweeteners with functional and sensory properties superior to those of many high-potency sweeteners.

This document describes materials and methods that can be used to produce steviol glycoside compositions, e.g., compositions enriched for particular steviol glycosides. Such compositions exhibit improved sensory profiles relative to compositions extracted from Stevia.

In one aspect, this document features a composition that includes at least 90% w/w rebaudioside A and food products that include such a composition. The composition has one or more of the following properties: a statistically significant decrease in a sweetness build score relative to a Stevia-derived rebaudioside A; a statistically significant decrease in an artificial sweetness score relative to a Stevia-derived rebaudioside A; a statistically significant decrease in a bitterness score relative to a Stevia-derived rebaudioside A; or a statistically significant decrease in two-minute acid score relative to a Stevia-derived rebaudioside A, where the scores determined in a standardized sensory panel evaluation. The rebaudioside A can be produced in a recombinant microorganism such as a yeast (e.g., Saccharomyces cerevisiae).

This document also features a method for producing a steviol glycoside product. The method includes fermenting a recombinant microorganism (e.g., Saccharomyces cerevisiae) capable of producing at least 1 g/L of the steviol glycoside in a culture medium or carrying out biocatalysis in a reaction mixture with one or more of the enzymes listed in Sections I-A, I-B, I-C, or I-D of the specification, to produce the steviol glycoside; and purifying the steviol glycoside from the culture medium or from the reaction mixture, using one or more purification steps selected from the group consisting of: (i) fractionation on an adsorbent resin; (ii) fractionation on a reversed phase resin; (iii) crystallization; and (iv) a drying step, thereby producing the steviol glycoside product having a statistically significant difference in at least one sensory attribute relative to a Stevia-derived steviol glycoside product. The sensory attribute evaluated in a standardized sensory panel evaluation. The steviol glycoside product can include at least 90% (e.g., at least 95% or 98%) w/w rebaudioside A. The method can include a crystallization step selected from the group consisting of anti-solvent crystallization, temperature-based crystallization, and evaporative crystallization. The method can include fractionation on a synthetic polyaromatic gel. The method can include fractionation on a reversed phase resin using medium pressure liquid chromatography.

In another aspect, this document features a composition comprising a non-plant-derived steviol glycoside with one or more improved taste characteristics as compared to a plant-derived preparation of the steviol glycoside, wherein the purity of the steviol glycoside is at least 90%.

Unless otherwise defined, all technical and scientific terms used herein have the same meaning as commonly understood by one of ordinary skill in the art to which the invention belongs. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and are not intended to be limiting. Other features and advantages of the invention will be apparent from the following detailed description. Applicants reserve the right to alternatively claim any disclosed invention using the transitional phrase “comprising,” “consisting essentially of,” or “consisting of,” according to standard practice in patent law.

### DESCRIPTION OF DRAWINGS

FIG. 1 shows the chemical structures of various steviol glycosides.

FIGS. 2A-D show representative pathways for the biosynthesis of steviol glycosides from steviol.

FIG. 3 shows a LC-ELSD-MS profile of crude fermentation broth.

FIG. 4 shows a LC-ELSD profile after HP-20, showing Reb A eluting at 10.3 minutes.

FIG. 5 shows a MPLC profile of fraction C-1713-11, showing Reb A eluting at 10.4 minutes.

FIG. 6 shows a MPLC profile of fraction C-1713-12, showing Reb A eluting at 10.4 minutes.

FIG. 7 shows a LC-MS-ELSD profile of fraction C-1713-11-12.

FIG. 8 shows an NMR plot of fraction C-1713-11-12.

FIG. 9 shows a spider plot showing sensory analysis profiles after the initial sip and after the third sip. * indicates a significant difference at 95% confidence level.

FIG. 10 is a spider plot showing sensory analysis profiles at the 20 second time point. * indicates a significant difference at 95% confidence level.

### DETAILED DESCRIPTION

### I. Steviol and Steviol Glycoside Biosynthesis Polypeptides

### A. Steviol Biosynthesis Polypeptides

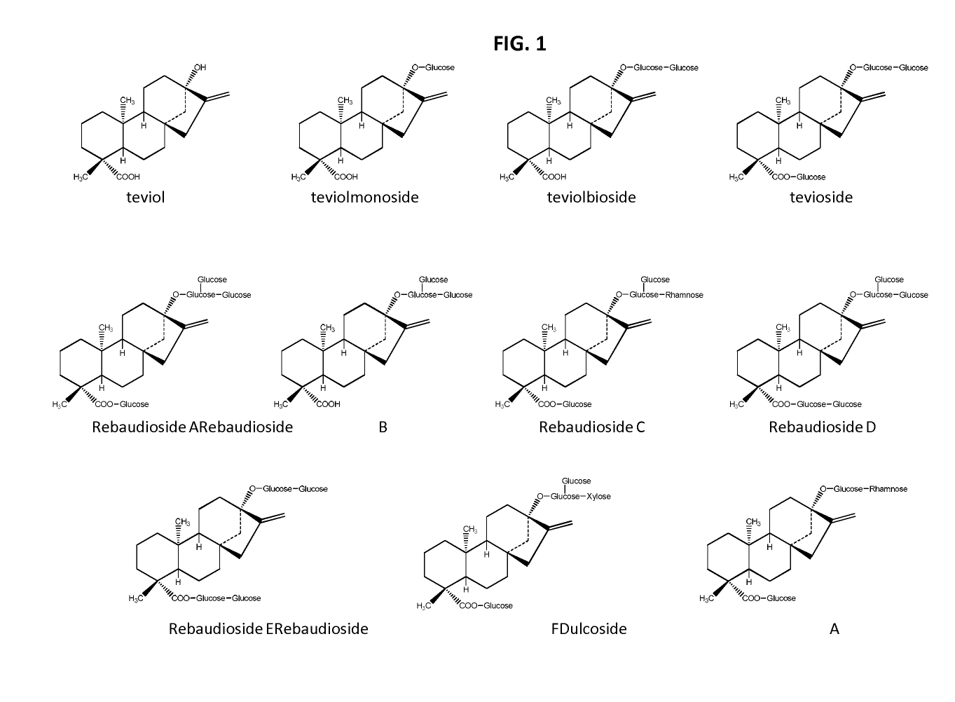

Chemical structures for several of the compounds found in Stevia extracts are shown in FIG. 1, including the diterpene steviol and various steviol glycosides. CAS numbers are shown in Table 1 below. See also, Steviol Glycosides Chemical and Technical Assessment 69th JECFA, prepared by Harriet Wallin, Food Agric. Org. (2007).

TABLE 1

| COMPOUND | CAS # |
|---|---|
| Steviol | 471-80-7 |
| Rebaudioside A | 58543-16-1 |
| Steviolbioside | 41093-60-1 |
| Stevioside | 57817-89-7 |
| Rebaudioside B | 58543-17-2 |
| Rebaudioside C | 63550-99-2 |
| Rebaudioside D | 63279-13-0 |
| Rebaudioside E | 63279-14-1 |
| Rebaudioside F | 438045-89-7 |
| Rubusoside | 63849-39-4 |
| Dulcoside A | 64432-06-0 |

It has been discovered that expression of certain genes in a microorganism confers the ability to synthesize steviol glycosides upon that host. As discussed in more detail below, one or more of such genes may be present naturally in a host. Typically, however, one or more of such genes are recombinant genes that have been transformed into a host that does not naturally possess them.

The biochemical pathway to produce steviol involves formation of geranylgeranyl diphosphate, cyclization to (−) copalyl diphosphate, followed by oxidation and hydroxylation to form steviol. Thus, conversion of geranylgeranyl diphosphate to steviol in a recombinant microorganism involves the expression of a gene encoding a kaurene synthase (KS), a gene encoding a kaurene oxidase (KO), and a gene encoding a steviol synthetase (KAH). Steviol synthetase also is known as kaurenoic acid 13-hydroxylase.

Suitable KS polypeptides are known. For example, suitable KS enzymes include those made by Stevia rebaudiana, Zea mays, Populus trichocarpa, and Arabidopsis thaliana. See, Table 2 and PCT Application Nos. PCT/US2012/050021 and PCT/US2011/038967, which are incorporated herein by reference in their entirety.

TABLE 2
KS Clones

| Enzyme Source | | Accession | Construct | Length |
|---|---|---|---|---|
| Organism | gi Number | Number | Name | (nts) |
| Stevia rebaudiana | 4959241 | AAD34295 | MM-12 | 2355 |
| Stevia rebaudiana | 4959239 | AAD34294 | MM-13 | 2355 |
| Zea mays | 162458963 | NP_001105097 | MM-14 | 1773 |
| Populus trichocarpa | 224098838 | XP_002311286 | MM-15 | 2232 |
| Arabidopsis thaliana | 3056724 | AF034774 | EV-70 | 2358 |

Suitable KO polypeptides are known. For example, suitable KO enzymes include those made by Stevia rebaudiana, Arabidopsis thaliana, Gibberella fujikoroi and Trametes versicolor. See, Table 3 and PCT Application Nos. PCT/US2012/050021 and PCT/US2011/038967, which are incorporated herein by reference in their entirety.

TABLE 3
KO Clones

| Enzyme Source | gi | Accession | Construct | Length |
|---|---|---|---|---|
| Organism | Number | Number | Name | (nts) |
| Stevia rebaudiana | 76446107 | ABA42921 | MM-18 | 1542 |
| Arabidopsis thaliana | 3342249 | AAC39505 | MM-19 | 1530 |
| Gibberella fujikoroi | 4127832 | CAA76703 | MM-20 | 1578 |
| Trametes versicolor | 14278967 | BAB59027 | MM-21 | 1500 |

Suitable KAH polypeptides are known. For example, suitable KAH enzymes include those made by Stevia rebaudiana, Arabidopsis thaliana, Vitis vinifera and Medicago trunculata. See, e.g., Table 4, PCT Application Nos. PCT/US2012/050021 and PCT/US2011/038967, U.S. Patent Publication Nos. 2008/0271205 and 2008/0064063, and Genbank Accession No. gi 189098312, which are incorporated herein by reference in their entirety. The steviol synthetase from Arabidopsis thaliana is classified as a CYP714A2.

TABLE 4
KAH Clones

| Enzyme | | | | | |
|---|---|---|---|---|---|
| Source | | Accession | Plasmid | Construct | Length |
| Organism | gi Number | Number | Name | Name | (nts) |
| Stevia | | —* | pMUS35 | MM-22 | 1578 |
| rebaudiana | | | | | |
| Stevia | 189418962 | ACD93722 | pMUS36 | MM-23 | 1431 |
| rebaudiana | | | | | |
| Arabidopsis | 15238644 | NP_197872 | pMUS37 | MM-24 | 1578 |
| thaliana | | | | | |
| Vitis vinifera | 225458454 | XP_002282091 | pMUS38 | MM-25 | 1590 |
| Medicago | 84514135 | ABC59076 | pMUS39 | MM-26 | 1440 |
| trunculata | | | | | |

* = Sequence is shown in U.S. patent Publication No. 2008-0064063.

In addition, a KAH polypeptide from Stevia rebaudiana that was identified as described in PCT Application No. PCT/US2012/050021 is particularly useful in a recombinant host. Nucleotide sequences encoding S. rebaudiana KAH (SrKAHe1) and S. rebaudiana KAH that has been codon-optimized for expression in yeast are set forth in the same PCT application, as is the encoded amino acid sequence of the S. rebaudiana KAH. The S. rebaudiana KAH shows significantly higher steviol synthase activity as compared to the Arabidopsis thaliana ent-kaurenoic acid hydroxylase described by Yamaguchi et al. (U.S. Patent Publication No. 2008/0271205 A1) when expressed in S. cerevisiae. The S. rebaudiana KAH polypeptide has less than 20% identity to the KAH from U.S. Patent Publication No. 2008/0271205, and less than 35% identity to the KAH from U.S. Patent Publication No. 2008/0064063.

In some embodiments, a recombinant microorganism contains a recombinant gene encoding a KO and/or a KAH polypeptide. Such microorganisms also typically contain a recombinant gene encoding a cytochrome P450 reductase (CPR) polypeptide, since certain combinations of KO and/or KAH polypeptides require expression of an exogenous CPR polypeptide. In particular, the activity of a KO and/or a KAH polypeptide of plant origin can be significantly increased by the inclusion of a recombinant gene encoding an exogenous CPR polypeptide. Suitable CPR polypeptides are known. For example, suitable CPR enzymes include those made by Stevia rebaudiana and Arabidopsis thaliana. See, e.g., Table 5 and PCT Application Nos. PCT/US2012/050021 and PCT/US2011/038967, which are incorporated herein by reference in their entirety.

TABLE 5
CPR Clones

| Enzyme Source | gi | Accession | Plasmid | Construct | Length |
|---|---|---|---|---|---|
| Organism | Number | Number | Name | Name | (nts) |
| Stevia | 93211213 | ABB88839 | pMUS40 | MM-27 | 2133 |
| rebaudiana | | | | | |
| Arabidopsis | 15233853 | NP_194183 | pMUS41 | MM-28 | 2079 |
| thaliana | | | | | |
| Giberella | 32562989 | CAE09055 | pMUS42 | MM-29 | 2142 |
| fujikuroi | | | | | |

For example, the steviol synthase encoded by SrKAHe1 is activated by the S. cerevisiae CPR encoded by gene NCP1 (YHR042W). Even better activation of the steviol synthase encoded by SrKAHe1 is observed when the Arabidopsis thaliana CPR encoded by the gene ATR2 or the S. rebaudiana CPR encoded by the gene CPR8 are co-expressed. Amino acid sequence of the S. cerevisiae, A. thaliana (from ATR1 and ATR2 genes) and S. rebaudiana CPR polypeptides (from CPR7 and CPR8 genes) are shown in PCT Application No. PCT/US2012/050021.

For example, the yeast gene DPP1 and/or the yeast gene LPP1 can be disrupted or deleted such that the degradation of farnesyl pyrophosphate (FPP) to farnesol is reduced and the degradation of geranylgeranylpyrophosphate (GGPP)) to geranylgeraniol (GGOH) is reduced. Alternatively, the promoter or enhancer elements of an endogenous gene encoding a phosphatase can be altered such that the expression of their encoded proteins is altered. Homologous recombination can be used to disrupt an endogenous gene. For example, a “gene replacement” vector can be constructed in such a way to include a selectable marker gene. The selectable marker gene can be operably linked, at both 5′ and 3′ end, to portions of the gene of sufficient length to mediate homologous recombination. The selectable marker can be one of any number of genes that complement host cell auxotrophy, provide antibiotic resistance, or result in a color change. Linearized DNA fragments of the gene replacement vector then are introduced into the cells using methods well known in the art (see below). Integration of the linear fragments into the genome and the disruption of the gene can be determined based on the selection marker and can be verified by, for example, Southern blot analysis. Subsequent to its use in selection, a selectable marker can be removed from the genome of the host cell by, e.g., Cre-loxP systems (see, e.g., Gossen et al. Ann. Rev. Genetics 36:153-173 (2002); and U.S. Publication No. 2006/0014264). Alternatively, a gene replacement vector can be constructed in such a way as to include a portion of the gene to be disrupted, where the portion is devoid of any endogenous gene promoter sequence and encodes none, or an inactive fragment of, the coding sequence of the gene. An “inactive fragment” is a fragment of the gene that encodes a protein having, e.g., less than about 10% (e.g., less than about 9%, less than about 8%, less than about 7%, less than about 6%, less than about 5%, less than about 4%, less than about 3%, less than about 2%, less than about 1%, or 0%) of the activity of the protein produced from the full-length coding sequence of the gene. Such a portion of the gene is inserted in a vector in such a way that no known promoter sequence is operably linked to the gene sequence, but that a stop codon and a transcription termination sequence are operably linked to the portion of the gene sequence. This vector can be subsequently linearized in the portion of the gene sequence and transformed into a cell. By way of single homologous recombination, this linearized vector is then integrated in the endogenous counterpart of the gene.

Expression in a recombinant microorganism of these genes results in the conversion of geranylgeranyl diphosphate to steviol.

### B. Steviol Glycoside Biosynthesis Polypeptides

A recombinant host described herein can convert steviol to a steviol glycoside. Such a host (e.g., microorganism) contains genes encoding one or more UDP Glycosyl Transferases, also known as UGTs. UGTs transfer a monosaccharide unit from an activated nucleotide sugar to an acceptor moiety, in this case, an —OH or —COOH moiety on steviol or steviol derivative. UGTs have been classified into families and subfamilies based on sequence homology. Li et al. J. Biol. Chem. 276:4338-4343 (2001).

### B.1 Rubusoside Biosynthesis Polypeptides

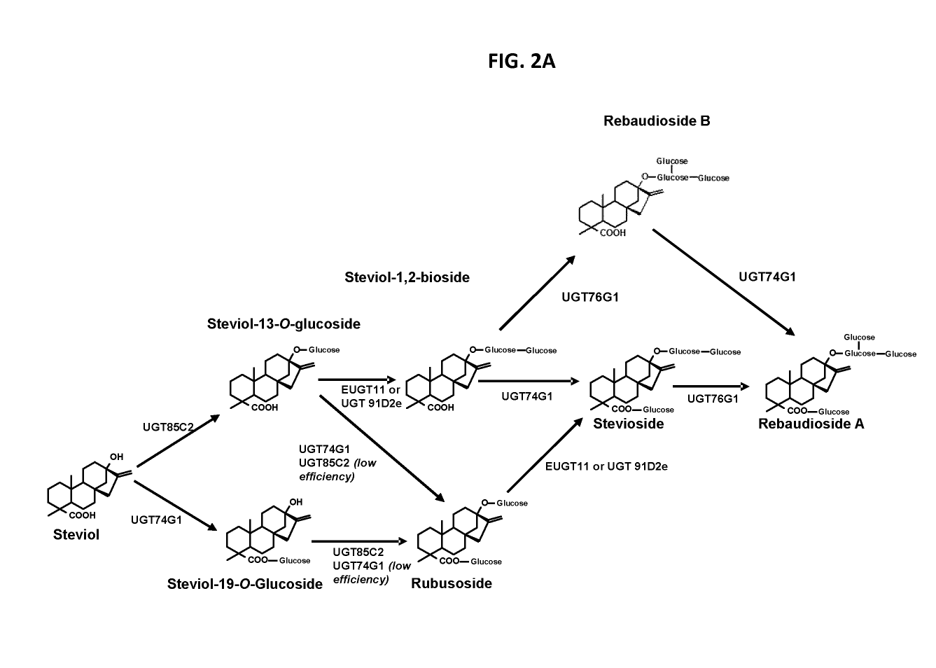

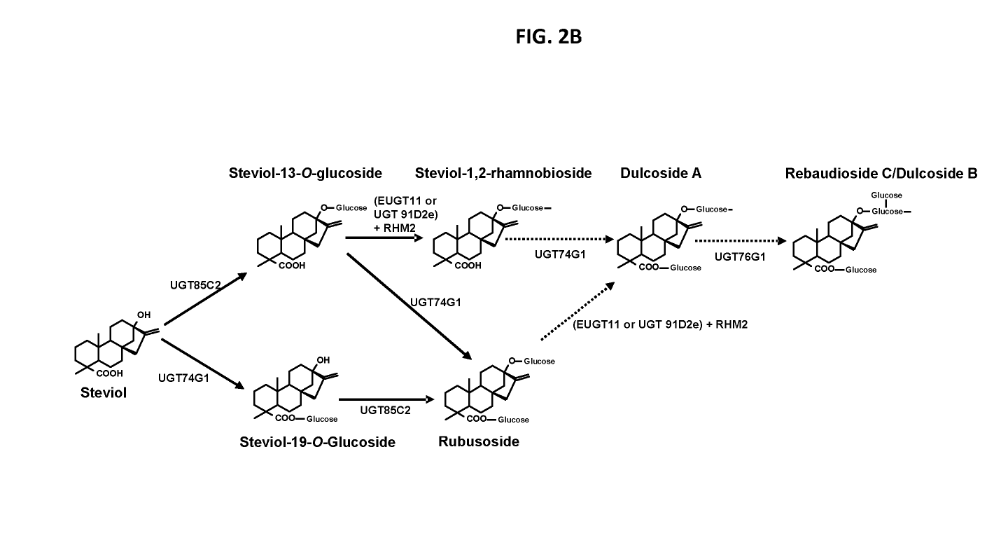

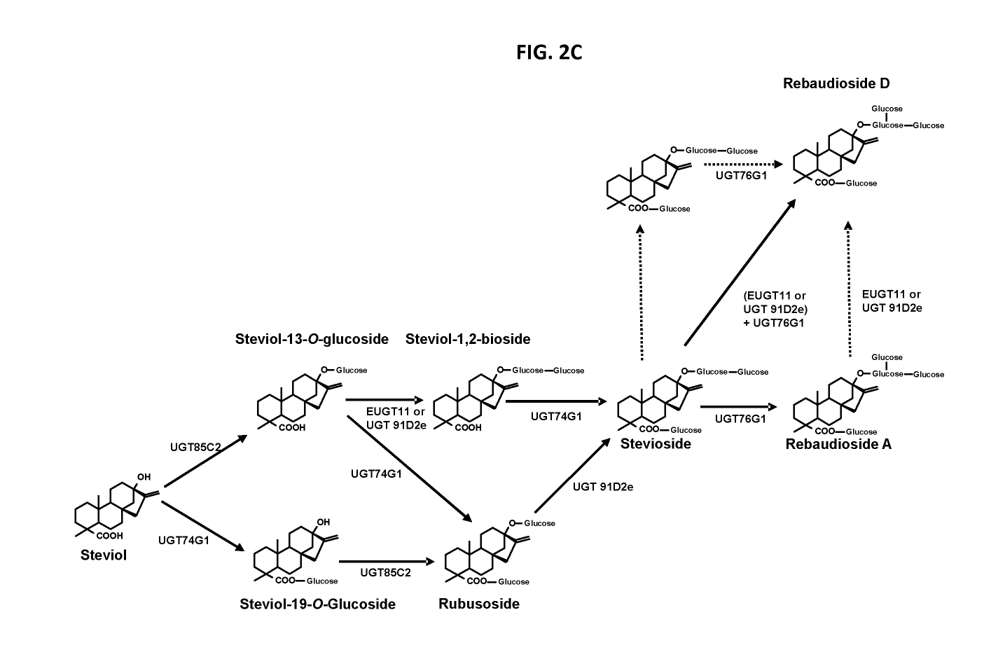

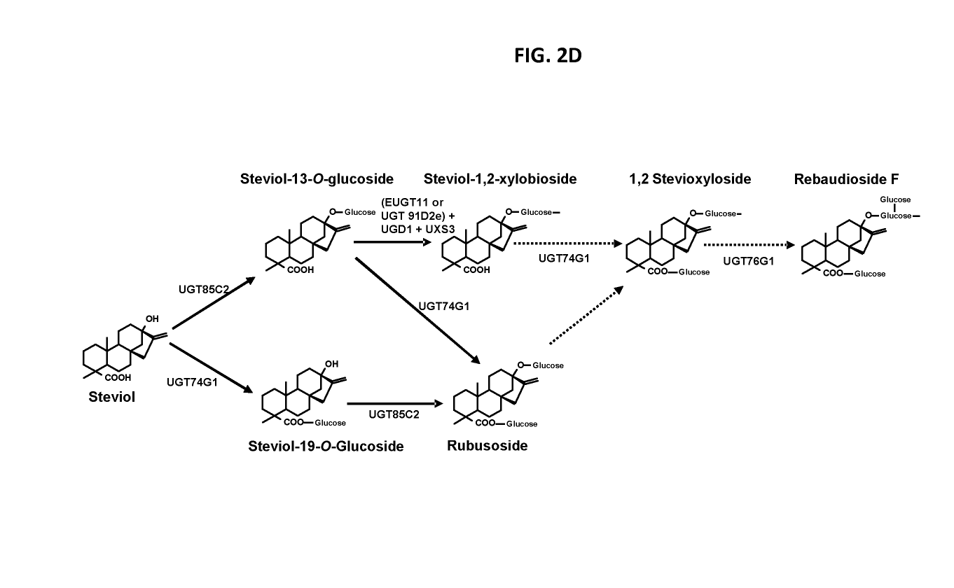

The biosynthesis of rubusoside involves glycosylation of the 13-OH and the 19-COOH of steviol. See FIG. 2A. Conversion of steviol to rubusoside in a recombinant host such as a microorganism can be accomplished by the expression of gene(s) encoding UGTs 85C2 and 74G1, which transfer a glucose unit to the 13-OH or the 19-COOH, respectively, of steviol.

A suitable UGT85C2 functions as a uridine 5′-diphospho glucosyl:steviol 13-OH transferase, and a uridine 5′-diphospho glucosyl:steviol-19-O-glucoside 13-OH transferase. Functional UGT85C2 polypeptides also may catalyze glucosyl transferase reactions that utilize steviol glycoside substrates other than steviol and steviol-19-O-glucoside.

A suitable UGT74G1 polypeptide functions as a uridine 5′-diphospho glucosyl: steviol 19-COOH transferase and a uridine 5′-diphospho glucosyl: steviol-13-O-glucoside 19-COOH transferase. Functional UGT74G1 polypeptides also may catalyze glycosyl transferase reactions that utilize steviol glycoside substrates other than steviol and steviol-13-O-glucoside, or that transfer sugar moieties from donors other than uridine diphosphate glucose.

A recombinant microorganism expressing a functional UGT74G1 and a functional UGT85C2 can make rubusoside and both steviol monosides (i.e., steviol 13-O-monoglucoside and steviol 19-O-monoglucoside) when steviol is used as a feedstock in the medium. One or more of such genes may be present naturally in the host. Typically, however, such genes are recombinant genes that have been transformed into a host (e.g., microorganism) that does not naturally possess them.

As used herein, the term recombinant host is intended to refer to a host, the genome of which has been augmented by at least one incorporated DNA sequence. Such DNA sequences include but are not limited to genes that are not naturally present, DNA sequences that are not normally transcribed into RNA or translated into a protein (“expressed”), and other genes or DNA sequences which one desires to introduce into the non-recombinant host. It will be appreciated that typically the genome of a recombinant host described herein is augmented through the stable introduction of one or more recombinant genes. Generally, the introduced DNA is not originally resident in the host that is the recipient of the DNA, but it is within the scope of the invention to isolate a DNA segment from a given host, and to subsequently introduce one or more additional copies of that DNA into the same host, e.g., to enhance production of the product of a gene or alter the expression pattern of a gene. In some instances, the introduced DNA will modify or even replace an endogenous gene or DNA sequence by, e.g., homologous recombination or site-directed mutagenesis. Suitable recombinant hosts include microorganisms.

The term “recombinant gene” refers to a gene or DNA sequence that is introduced into a recipient host, regardless of whether the same or a similar gene or DNA sequence may already be present in such a host. “Introduced,” or “augmented” in this context, is known in the art to mean introduced or augmented by the hand of man. Thus, a recombinant gene may be a DNA sequence from another species, or may be a DNA sequence that originated from or is present in the same species, but has been incorporated into a host by recombinant methods to form a recombinant host. It will be appreciated that a recombinant gene that is introduced into a host can be identical to a DNA sequence that is normally present in the host being transformed, and is introduced to provide one or more additional copies of the DNA to thereby permit overexpression or modified expression of the gene product of that DNA.

Suitable UGT74G1 and UGT85C2 polypeptides include those made by Stevia rebaudiana. Genes encoding functional UGT74G1 and UGT85C2 polypeptides from Stevia are reported in Richman et al. Plant J. 41: 56-67 (2005). Amino acid sequences of S. rebaudiana UGT74G1 and UGT85C2 polypeptides are set forth in SEQ ID NOs: 1 and 3, respectively, of PCT Application No. PCT/US2012/050021, as are nucleotide sequences that encode UGT74G1 and UGT85C2 and that have been optimized for expression in yeast, and DNA 2.0 codon-optimized sequence for UGTs 85C2, 91 D2e, 74G1 and 76G1. See also the UGT85C2 and UGT74G1 variants described below in the “Functional Homolog” section. For example, a UGT85C2 polypeptide can contain substitutions at positions 65, 71, 270, 289, and 389 can be used (e.g., A65S, E71Q, T270M, Q289H, and A389V).

In some embodiments, the recombinant host is a microorganism. The recombinant microorganism can be grown on media containing steviol in order to produce rubusoside. In other embodiments, however, the recombinant microorganism expresses one or more recombinant genes involved in steviol biosynthesis, e.g., a CDPS gene, a KS gene, a KO gene and/or a KAH gene. Suitable CDPS polypeptides are known. For example, suitable CDPS enzymes include those made by Stevia rebaudiana, Streptomyces clavuligerus, Bradyrhizobium japonicum, Zea mays, and Arabidopsis. See, e.g., Table 6 and PCT Application Nos. PCT/US2012/050021 and PCT/US2011/038967, which are incorporated herein by reference in their entirety.

In some embodiments, CDPS polypeptides that lack a chloroplast transit peptide at the amino terminus of the unmodified polypeptide can be used. For example, the first 150 nucleotides from the 5′ end of the Zea mays CDPS coding sequence shown in FIG. 14 of PCT Publication No. PCT/US2012/050021 can be removed. Doing so removes the amino terminal 50 residues of the amino acid sequence, which encode a chloroplast transit peptide. The truncated CDPS gene can be fitted with a new ATG translation start site and operably linked to a promoter, typically a constitutive or highly expressing promoter. When a plurality of copies of the truncated coding sequence are introduced into a microorganism, expression of the CDPS polypeptide from the promoter results in an increased carbon flux towards ent-kaurene biosynthesis.

TABLE 6
CDPS Clones

| Enzyme Source | | Accession | Plasmid | Construct | Length |
|---|---|---|---|---|---|
| Organism | gi Number | Number | Name | Name | (nts) |
| Stevia | 2642661 | AAB87091 | pMUS22 | MM-9 | 2364 |
| rebaudiana | | | | | |
| Streptomyces | 197705855 | EDY51667 | pMUS23 | MM-10 | 1584 |
| cla vuligerus | | | | | |
| Bradyrhizobium | 529968 | AAC28895.1 | pMUS24 | MM-11 | 1551 |
| japonicum | | | | | |
| Zea mays | 50082774 | AY562490 | | EV65 | 2484 |
| Arabidopsis | 18412041 | NM_116512 | | EV64 | 2409 |
| thaliana | | | | | |

CDPS-KS bifunctional proteins also can be used. Nucleotide sequences encoding the CDPS-KS bifunctional enzymes shown in Table 7 were modified for expression in yeast (see PCT Application Nos. PCT/US2012/050021). A bifunctional enzyme from Gibberella fujikuroi also can be used.

TABLE 7
CDPS-KS Clones

| Enzyme Source | | Accession | Construct | Length |
|---|---|---|---|---|
| Organism | gi Number | Number | Name | (nts) |
| Phomopsis amygdali | 186704306 | BAG30962 | MM-16 | 2952 |
| Physcomitrella patens | 146325986 | BAF61135 | MM-17 | 2646 |
| Gibberella fujikuroi | 62900107 | Q9UVY5.1 | | 2859 |

Thus, a microorganism containing a CDPS gene, a KS gene, a KO gene and a KAH gene in addition to a UGT74G1 and a UGT85C2 gene is capable of producing both steviol monosides and rubusoside without the necessity for using steviol as a feedstock.

In some embodiments, the recombinant microorganism further expresses a recombinant gene encoding a geranylgeranyl diphosphate synthase (GGPPS). Suitable GGPPS polypeptides are known. For example, suitable GGPPS enzymes include those made by Stevia rebaudiana, Gibberella fujikuroi, Mus musculus, Thalassiosira pseudonana, Streptomyces clavuligerus, Sulfulobus acidocaldarius, Synechococcus sp. and Arabidopsis thaliana. See, Table 8 and PCT Application Nos. PCT/US2012/050021 and PCT/US2011/038967, which are incorporated herein by reference in their entirety.

TABLE 8
GGPPS Clones

| Enzyme | | | | Con- | |
|---|---|---|---|---|---|
| Source | | Accession | Plasmid | struct | Length |
| Organism | gi Number | Number | Name | Name | (nts) |
| Stevia | 90289577 | ABD92926 | pMUS14 | MM-1 | 1086 |
| rebaudiana | | | | | |
| Gibberella | 3549881 | CAA75568 | pMUS15 | MM-2 | 1029 |
| fujikuroi | | | | | |
| Mus musculus | 47124116 | AAH69913 | pMUS16 | MM-3 | 903 |
| Thalassiosira | 223997332 | XP_002288339 | pMUS17 | MM-4 | 1020 |
| pseudonana | | | | | |
| Streptomyces | 254389342 | ZP_05004570 | pMUS18 | MM-5 | 1068 |
| clavuligerus | | | | | |
| Sulfulobus | 506371 | BAA43200 | pMUS19 | MM-6 | 993 |
| acidocaldarius | | | | | |
| Synechococcus | 86553638 | ABC98596 | pMUS20 | MM-7 | 894 |
| sp. | | | | | |
| Arabidopsis | 15234534 | NP_195399 | pMUS21 | MM-8 | 1113 |
| thaliana | | | | | |

In some embodiments, the recombinant microorganism further can express recombinant genes involved in diterpene biosynthesis or production of terpenoid precursors, e.g., genes in the methylerythritol 4-phosphate (MEP) pathway or genes in the mevalonate (MEV) pathway discussed below, have reduced phosphatase activity, and/or express a sucrose synthase (SUS) as discussed herein.

### B.2 Rebaudioside A, Rebaudioside D, and Rebaudioside E Biosynthesis Polypeptides

The biosynthesis of rebaudioside A involves glucosylation of the aglycone steviol. Specifically, rebaudioside A can be formed by glucosylation of the 13-OH of steviol which forms the 13-O-steviolmonoside, glucosylation of the C-2′ of the 13-O-glucose of steviolmonoside which forms steviol-1,2-bioside, glucosylation of the C-19 carboxyl of steviol-1,2-bioside which forms stevioside, and glucosylation of the C-3′ of the C-13-O-glucose of stevioside. The order in which each glucosylation reaction occurs can vary. See FIG. 2A.

The biosynthesis of rebaudioside E and/or rebaudioside D involves glucosylation of the aglycone steviol. Specifically, rebaudioside E can be formed by glucosylation of the 13-OH of steviol which forms steviol-13-O-glucoside, glucosylation of the C-2′ of the 13-O-glucose of steviol-13-O-glucoside which forms the steviol-1,2-bioside, glucosylation of the C-19 carboxyl of the 1,2-bioside to form 1,2-stevioside, and glucosylation of the C-2′ of the 19-0-glucose of the 1,2-stevioside to form rebaudioside E. Rebaudioside D can be formed by glucosylation of the C-3′ of the C-13-O-glucose of rebaudioside E. The order in which each glycosylation reaction occurs can vary. For example, the glucosylation of the C-2′ of the 19-0-glucose may be the last step in the pathway, wherein Rebaudioside A is an intermediate in the pathway. See FIG. 2C.

It has been discovered that conversion of steviol to rebaudioside A, rebaudioside D, and/or rebaudioside E in a recombinant host can be accomplished by expressing the following functional UGTs: EUGT11, 74G1, 85C2, and 76G1, and optionally 91D2. Thus, a recombinant microorganism expressing combinations of these four or five UGTs can make rebaudioside A and rebaudioside D when steviol is used as a feedstock. Typically, one or more of these genes are recombinant genes that have been transformed into a microorganism that does not naturally possess them. It has also been discovered that UGTs designated herein as SM12UGT can be substituted for UGT91 D2.

In some embodiments, less than five (e.g., one, two, three, or four) UGTs are expressed in a host. For example, a recombinant microorganism expressing a functional EUGT11 can make rebaudioside D when rebaudioside A is used as a feedstock. A recombinant microorganism expressing two functional UGTs, EUGT11 and 76G1, and optionally a functional 91D12, can make rebaudioside D when rubusoside or 1,2-stevioside is used as a feedstock. As another alternative, a recombinant microorganism expressing three functional UGTs, EUGT11, 74G1, 76G1, and optionally 91D2, can make rebaudioside D when fed the monoside, steviol-13-O-glucoside, in the medium. Similarly, conversion of steviol-19-O-glucoside to rebaudioside D in a recombinant microorganism can be accomplished by the expression of genes encoding UGTs EUGT11, 85C2, 76G1, and optionally 91D2, when fed steviol-19-O-glucoside. Typically, one or more of these genes are recombinant genes that have been transformed into a host that does not naturally possess them.

Suitable UGT74G1 and UGT85C2 polypeptides include those discussed above. A suitable UGT76G1 adds a glucose moiety to the C-3′ of the C-13-O-glucose of the acceptor molecule, a steviol 1,2 glycoside. Thus, UGT76G1 functions, for example, as a uridine 5′-diphospho glucosyl: steviol 13-O-1,2 glucoside C-3′ glucosyl transferase and a uridine 5′-diphospho glucosyl: steviol-19-O-glucose, 13-O-1,2 bioside C-3′ glucosyl transferase. Functional UGT76G1 polypeptides may also catalyze glucosyl transferase reactions that utilize steviol glycoside substrates that contain sugars other than glucose, e.g., steviol rhamnosides and steviol xylosides. See, FIGS. 2A, 2B, 2C and 2D. Suitable UGT76G1 polypeptides include those made by S. rebaudiana and reported in Richman et al. Plant J. 41: 56-67 (2005). The amino acid sequence of a S. rebaudiana UGT76G1 polypeptide is set forth in PCT Publication No. PCT/US2012/050021, as is a nucleotide sequence that encodes the UGT76G1 polypeptide and is optimized for expression in yeast. See also the UGT76G1 variants set forth in the “Functional Homolog” section.

A suitable EUGT11 or UGT91D2 polypeptide functions as a uridine 5′-diphospho glucosyl: steviol-13-O-glucoside transferase (also referred to as a steviol-13-monoglucoside 1,2-glucosylase), transferring a glucose moiety to the C-2′ of the 13-O-glucose of the acceptor molecule, steviol-13-O-glucoside.

A suitable EUGT11 or UGT91D2 polypeptide also functions as a uridine 5′-diphospho glucosyl: rubusoside transferase transferring a glucose moiety to the C-2′ of the 13-O-glucose of the acceptor molecule, rubusoside, to produce stevioside. EUGT11 polypeptides also can transfer a glucose moiety to the C-2′ of the 19-O-glucose of the acceptor molecule, rubusoside, to produce a 19-O-1,2-diglycosylated rubusoside.

Functional EUGT11 or UGT91D2 polypeptides also can catalyze reactions that utilize steviol glycoside substrates other than steviol-13-O-glucoside and rubusoside. For example, a functional EUGT11 polypeptide may utilize stevioside as a substrate, transferring a glucose moiety to the C-2′ of the 19-O-glucose residue to produce Rebaudioside E. Functional EUGT11 and UGT91D2 polypeptides may also utilize Rebaudioside A as a substrate, transferring a glucose moiety to the C-2′ of the 19-O-glucose residue of Rebaudioside A to produce Rebaudioside D. EUGT11 can convert Rebaudioside A to Rebaudioside D at a rate that is least 20 times faster (e.g., as least 25 times or at least 30 times faster) than the corresponding rate of UGT91 D2e (SEQ ID NO: 5 of PCT Application N. PCT/US2012/050021) when the reactions are performed under similar conditions, i.e., similar time, temperature, purity, and substrate concentration. As such, EUGT11 produces greater amounts of RebD than UGT91 D2e when incubated under similar conditions.

In addition, a functional EUGT11 exhibits significant C-2′ 19-O-diglycosylation activity with rubusoside or stevioside as substrates, whereas UGT91D2e has no detectable diglycosylation activity with these substrates. Thus, a functional EUGT11 can be distinguished from UGT91 D2e by the differences in steviol glycoside substrate-specificity.

A functional EUGT11 or UGT91 D2 polypeptide typically does not transfer a glucose moiety to steviol compounds having a 1,3-bound glucose at the C-13 position, i.e., transfer of a glucose moiety to steviol 1,3-bioside and 1,3-stevioside does not occur.

Functional EUGT11 and UGT91D2 polypeptides can transfer sugar moieties from donors other than uridine diphosphate glucose. For example, a functional EUGT11 or UGT91D2 polypeptide can act as a uridine 5′-diphospho D-xylosyl: steviol-13-O-glucoside transferase, transferring a xylose moiety to the C-2′ of the 13-O-glucose of the acceptor molecule, steviol-13-O-glucoside. As another example, a functional EUGT11 or UGT91D2 polypeptide can act as a uridine 5′-diphospho L-rhamnosyl: steviol-13-O-glucoside transferase, transferring a rhamnose moiety to the C-2′ of the 13-O-glucose of the acceptor molecule, steviol-13-O-glucoside

Suitable EUGT11 polypeptides can include the EUGT11 polypeptide from Oryza sativa (GenBank Accession No. AC133334). For example, an EUGT11 polypeptide can have an amino acid sequence with at least 70% sequence identity (e.g., at least 75, 80, 85, 90, 95, 96, 97, 98, or 99% sequence identity) to the amino acid sequence set forth in SEQ ID NO:152 of PCT Application No. PCT/US2012/050021.

Suitable functional UGT91D2 polypeptides include those disclosed herein, e.g., the polypeptides designated UGT91 D2e and UGT91 D2m. The amino acid sequence of an exemplary UGT91D2e polypeptide from Stevia rebaudiana is set forth in SEQ ID NO: 5 of PCT Application No. PCT/US2012/050021, which also discloses the S. rebaudiana nucleotide sequence encoding the polypeptide, a nucleotide sequence that encodes the polypeptide and that has been codon optimized for expression in yeast, the amino acid sequences of exemplary UGT91D2m polypeptides from S. rebaudiana, and nucleic acid sequences encoding the exemplary UGT91D2m polypeptides. UGT91D2 variants containing a substitution at amino acid residues 206, 207, and 343 also can be used. For example, the amino acid sequence having G206R, Y207C, and W343R mutations with respect to wild-type UGT92D2e can be used. In addition, a UGT91D2 variant containing substitutions at amino acid residues 211 and 286 can be used. For example, a UGT91 D2 variant can include a substitution of a methionine for leucine at position 211 and a substitution of an alanine for valine at position 286.

As indicated above, UGTs designated herein as SM12UGT can be substituted for UGT91D2. Suitable functional SM12UGT polypeptides include those made by Ipomoea purpurea (Japanese morning glory) and described in Morita et al. Plant J. 42: 353-363 (2005). The amino acid sequence encoding the I. purpurea IP3GGT polypeptide is set forth in PCT Application No. PCT/US2012/050021, as is a nucleotide sequence that encodes the polypeptide and that has been codon optimized for expression in yeast. Another suitable SM12UGT polypeptide is a Bp94B1 polypeptide having an R25S mutation. See Osmani et al. Plant Phys. 148: 1295-1308 (2008) and Sawada et al. J. Biol. Chem. 280: 899-906 (2005). The amino acid sequence of the Bellis perennis (red daisy) UGT94B1 polypeptide is set forth in PCT Application No. PCT/US2012/050021, as is a nucleotide sequence that encodes the polypeptide and that has been codon optimized for expression in yeast.

In some embodiments, the recombinant microorganism is grown on media containing steviol-13-O-glucoside or steviol-19-O-glucoside in order to produce rebaudioside A and/or rebaudioside D. In such embodiments, the microorganism contains and expresses genes encoding a functional EUGT11, a functional UGT74G1, a functional UGT85C2, a functional UGT76G1, and an optional functional UGT91D2, and is capable of accumulating rebaudioside A and rebaudioside D when steviol, one or both of the steviolmonosides, or rubusoside is used as feedstock.

In other embodiments, the recombinant microorganism is grown on media containing rubusoside in order to produce rebaudioside A and/or rebaudioside D. In such embodiments, the microorganism contains and expresses genes encoding a functional EUGT11, a functional UGT76G1, and an optional functional UGT91D2, and is capable of producing rebaudioside A and/or rebaudioside D when rubusoside is used as feedstock.

In other embodiments the recombinant microorganism expresses one or more genes involved in steviol biosynthesis, e.g., a CDPS gene, a KS gene, a KO gene and/or a KAH gene. Thus, for example, a microorganism containing a CDPS gene, a KS gene, a KO gene and a KAH gene, in addition to a EUGT11, a UGT74G1, a UGT85C2, a UGT76G1, and optionally a functional UGT91 D2 (e.g., UGT91 D2e), is capable of producing rebaudioside A, rebaudioside D, and/or rebaudioside E without the necessity for including steviol in the culture media.

In some embodiments, the recombinant host further contains and expresses a recombinant GGPPS gene in order to provide increased levels of the diterpene precursor geranylgeranyl diphosphate, for increased flux through the steviol biosynthetic pathway. In some embodiments, the recombinant host further contains a construct to silence the expression of non-steviol pathways consuming geranylgeranyl diphosphate, ent-Kaurenoic acid or farnesyl pyrophosphate, thereby providing increased flux through the steviol and steviol glycosides biosynthetic pathways. For example, flux to sterol production pathways such as ergosterol may be reduced by downregulation of the ERG9 gene. In cells that produce gibberellins, gibberellin synthesis may be downregulated to increase flux of ent-kaurenoic acid to steviol. In carotenoid-producing organisms, flux to steviol may be increased by downregulation of one or more carotenoid biosynthetic genes. In some embodiments, the recombinant microorganism further can express recombinant genes involved in diterpene biosynthesis or production of terpenoid precursors, e.g., genes in the MEP or MEV) pathways, have reduced phosphatase activity, and/or express a SUS.

One with skill in the art will recognize that by modulating relative expression levels of different UGT genes, a recombinant host can be tailored to specifically produce steviol glycoside products in a desired proportion. Transcriptional regulation of steviol biosynthesis genes and steviol glycoside biosynthesis genes can be achieved by a combination of transcriptional activation and repression using techniques known to those in the art. For in vitro reactions, one with skill in the art will recognize that addition of different levels of UGT enzymes in combination or under conditions which impact the relative activities of the different UGTS in combination will direct synthesis towards a desired proportion of each steviol glycoside. One with skill in the art will recognize that a higher proportion of rebaudioside D or E or more efficient conversion to rebaudioside D or E can be obtained with a diglycosylation enzyme that has a higher activity for the 19-O-glucoside reaction as compared to the 13-O-glucoside reaction (substrates rebaudioside A and stevioside).

In some embodiments, a recombinant host such as a microorganism produces rebaudioside D-enriched steviol glycoside compositions that have greater than at least 3% rebaudioside D by weight total steviol glycosides, e.g., at least 4% rebaudioside D at least 5% rebaudioside D, 10-20% rebaudioside D, 20-30% rebaudioside D, 30-40% rebaudioside D, 40-50% rebaudioside D, 50-60% rebaudioside D, 60-70% rebaudioside D, 70-80% rebaudioside D. In some embodiments, a recombinant host such as a microorganism produces steviol glycoside compositions that have at least 90% rebaudioside D, e.g., 90-99% rebaudioside D. Other steviol glycosides present may include those depicted in FIG. 2C such as steviol monosides, steviol glucobiosides, rebaudioside A, rebaudioside E, and stevioside. In some embodiments, the rebaudioside D-enriched composition produced by the host (e.g., microorganism) can be further purified and the rebaudioside D or rebaudioside E so purified can then be mixed with other steviol glycosides, flavors, or sweeteners to obtain a desired flavor system or sweetening composition. For instance, a rebaudioside D-enriched composition produced by a recombinant host can be combined with a rebaudioside A, C, or F-enriched composition produced by a different recombinant host, with rebaudioside A, F, or C purified from a Stevia extract, or with rebaudioside A, F, or C produced in vitro.

In some embodiments, rebaudioside A, rebaudioside D, rebaudioside B, steviol monoglucosides, steviol-1,2-bioside, rubusoside, stevioside, or rebaudioside E can be produced using in vitro methods while supplying the appropriate UDP-sugar and/or a cell-free system for regeneration of UDP-sugars. See, for example, Jewett et al. Molecular Systems Biology, Vol. 4, article 220 (2008); Masada et al. FEBS Lett. 581: 2562-2566 (2007). In some embodiments, sucrose and a sucrose synthase may be provided in the reaction vessel in order to regenerate UDP-glucose from the UDP generated during glycosylation reactions. The sucrose synthase can be from any suitable organism. For example, a sucrose synthase coding sequence from Arabidopsis thaliana, Stevia rebaudiana, or Coffea arabica can be cloned into an expression plasmid under control of a suitable promoter, and expressed in a host such as a microorganism.

Conversions requiring multiple reactions may be carried out together, or stepwise. For example, rebaudioside D may be produced from rebaudioside A that is commercially available as an enriched extract or produced via biosynthesis, with the addition of stoichiometric or excess amounts of UDP-glucose and EUGT11. As an alternative, rebaudioside D may be produced from steviol glycoside extracts that are enriched for stevioside and rebaudioside A, using EUGT11 and a suitable UGT76G1 enzyme. In some embodiments, phosphatases are used to remove secondary products and improve the reaction yields. UGTs and other enzymes for in vitro reactions may be provided in soluble forms or in immobilized forms.

In some embodiments, rebaudioside A, rebaudioside D, or rebaudioside E can be produced using whole cells that are fed raw materials that contain precursor molecules such as steviol and/or steviol glycosides, including mixtures of steviol glycosides derived from plant extracts. The raw materials may be fed during cell growth or after cell growth. The whole cells may be in suspension or immobilized. The whole cells may be entrapped in beads, for example calcium or sodium alginate beads. The whole cells may be linked to a hollow fiber tube reactor system. The whole cells may be concentrated and entrapped within a membrane reactor system. The whole cells may be in fermentation broth or in a reaction buffer. In some embodiments, a permeabilizing agent is utilized for efficient transfer of substrate into the cells. In some embodiments, the cells are permeabilized with a solvent such as toluene, or with a detergent such as Triton-X or Tween. In some embodiments, the cells are permeabilized with a surfactant, for example a cationic surfactant such as cetyltrimethylammonium bromide (CTAB). In some embodiments, the cells are permeabilized with periodic mechanical shock such as electroporation or a slight osmotic shock. The cells can contain one recombinant UGT or multiple recombinant UGTs. For example, the cells can contain UGT 76G1 and EUGT11 such that mixtures of stevioside and RebA are efficiently converted to RebD. In some embodiments, the whole cells are the host cells described in section III A. In some embodiments, the whole cells are a Gram-negative bacterium such as E. coli. In some embodiments, the whole cell is a Gram-positive bacterium such as Bacillus. In some embodiments, the whole cell is a fungal species such as Aspergillus, or a yeast such as Saccharomyces. In some embodiments, the term “whole cell biocatalysis” is used to refer to the process in which the whole cells are grown as described above (e.g., in a medium and optionally permeabilized) and a substrate such as rebA or stevioside is provided and converted to the end product using the enzymes from the cells. The cells may or may not be viable, and may or may not be growing during the bioconversion reactions. In contrast, in fermentation, the cells are cultured in a growth medium and fed a carbon and energy source such as glucose and the end product is produced with viable cells.

### B.3 Dulcoside A and Rebaudioside C Biosynthesis Polypeptides

The biosynthesis of rebaudioside C and/or dulcoside A involves glucosylation and rhamnosylation of the aglycone steviol. Specifically, dulcoside A can be formed by glucosylation of the 13-OH of steviol which forms steviol-13-O-glucoside, rhamnosylation of the C-2′ of the 13-O-glucose of steviol-13-O-glucoside which forms the 1,2 rhamnobioside, and glucosylation of the C-19 carboxyl of the 1,2 rhamnobioside. Rebaudioside C can be formed by glucosylation of the C-3′ of the C-13-O-glucose of dulcoside A. The order in which each glycosylation reaction occurs can vary. See FIG. 2B.

It has been discovered that conversion of steviol to dulcoside A in a recombinant host can be accomplished by the expression of gene(s) encoding the following functional UGTs: 85C2, EUGT11 and/or 91D2e, and 74G1. Thus, a recombinant microorganism expressing these three or four UGTs and a rhamnose synthetase can make dulcoside A when fed steviol in the medium. Alternatively, a recombinant microorganism expressing two UGTs, EUGT11 and 74G1, and rhamnose synthetase can make dulcoside A when fed the monoside, steviol-13-O-glucoside or steviol-19-O-glucoside, in the medium. Similarly, conversion of steviol to rebaudioside C in a recombinant microorganism can be accomplished by the expression of gene(s) encoding UGTs 85C2, EUGT11, 74G1, 76G1, optionally 91D2e, and rhamnose synthetase when fed steviol, by the expression of genes encoding UGTs EUGT11 and/or 91D2e, 74G1, and 76G1, and rhamnose synthetase when fed steviol-13-O-glucoside, by the expression of genes encoding UGTs 85C2, EUGT11 and/or 91D2e, 76G1, and rhamnose synthetase when fed steviol-19-O-glucoside, or by the expression of genes encoding UGTs EUGT11 and/or 91D2e, 76G1, and rhamnose synthetase when fed rubusoside. Typically, one or more of these genes are recombinant genes that have been transformed into a microorganism that does not naturally possess them.

Suitable EUGT11, UGT91D2, UGT74G1, UGT76G1 and UGT85C2 polypeptides include the functional UGT polypeptides discussed herein. Rhamnose synthetase provides increased amounts of the UDP-rhamnose donor for rhamnosylation of the steviol compound acceptor. Suitable rhamnose synthetases include those made by Arabidopsis thaliana, such as the product of the A. thaliana RHM2 gene.

In some embodiments, a UGT79B3 polypeptide is substituted for a UGT91D2 polypeptide. Suitable UGT79B3 polypeptides include those made by Arabidopsis thaliana, which are capable of rhamnosylation of steviol 13-O-monoside in vitro. A. thaliana UGT79B3 can rhamnosylate glucosylated compounds to form 1,2-rhamnosides. The amino acid sequence of an Arabidopsis thaliana UGT79B3 is set forth in PCT Application No. PCT/US2012/050021, as is a nucleotide sequence encoding the amino acid sequence.

In some embodiments, rebaudioside C can be produced using in vitro methods while supplying the appropriate UDP-sugar and/or a cell-free system for regeneration of UDP-sugars. See, for example, “An integrated cell-free metabolic platform for protein production and synthetic biology” by Jewett M C, Calhoun K A, Voloshin A, Wuu J J and Swartz J R in Molecular Systems Biology, 4, article 220 (2008); Masada et al. FEBS Lett. 581: 2562-2566 (2007). In some embodiments, sucrose and a sucrose synthase may be provided in the reaction vessel in order to regenerate UDP-glucose from UDP during the glycosylation reactions. The sucrose synthase can be from any suitable organism. For example, a sucrose synthase coding sequence from Arabidopsis thaliana, Stevia rebaudiana, or Coffea arabica can be cloned into an expression plasmid under control of a suitable promoter, and expressed in a microorganism. In some embodiments a RHM2 enzyme (Rhamnose synthase) may also be provided, with NADPH, to generate UDP-rhamnose from UDP-glucose.

Reactions may be carried out together, or stepwise. For instance, rebaudioside C may be produced from rubusoside with the addition of stoichiometric amounts of UDP-rhamnose and EUGT11, followed by addition of UGT76G1 and an excess or stoichiometric supply of UDP-glucose. In some embodiments, phosphatases are used to remove secondary products and improve the reaction yields. UGTs and other enzymes for in vitro reactions may be provided in soluble forms or immobilized forms. In some embodiments, rebaudioside C, Dulcoside A, or other steviol rhamnosides can be produced using whole cells as discussed above. The cells can contain one recombinant UGT or multiple recombinant UGTs. For example, the cells can contain UGT 76G1 and EUGT11 such that mixtures of stevioside and RebA are efficiently converted to RebD. In some embodiments, the whole cells are the host cells described in section III A.

In other embodiments, the recombinant host expresses one or more genes involved in steviol biosynthesis, e.g., a CDPS gene, a KS gene, a KO gene and/or a KAH gene. Thus, for example, a microorganism containing a CDPS gene, a KS gene, a KO gene and a KAH gene, in addition to a UGT85C2, a UGT74G1, a EUGT11 gene, optionally a UGT91D2e gene, and a UGT76G1 gene, is capable of producing rebaudioside C without the necessity for including steviol in the culture media. In addition, the recombinant host typically expresses an endogenous or a recombinant gene encoding a rhamnose synthetase. Such a gene is useful in order to provide increased amounts of the UDP-rhamnose donor for rhamnosylation of the steviol compound acceptor. Suitable rhamnose synthetases include those made by Arabidopsis thaliana, such as the product of the A. thaliana RHM2 gene.

One with skill in the art will recognize that by modulating relative expression levels of different UGT genes as well as modulating the availability of UDP-rhamnose, a recombinant host can be tailored to specifically produce steviol and steviol glycoside products in a desired proportion. Transcriptional regulation of steviol biosynthesis genes and steviol glycoside biosynthesis genes can be achieved by a combination of transcriptional activation and repression using techniques known to those in the art. For in vitro reactions, one with skill in the art will recognize that addition of different levels of UGT enzymes in combination or under conditions which impact the relative activities of the different UGTS in combination will direct synthesis towards a desired proportion of each steviol glycoside.

In some embodiments, the recombinant host further contains and expresses a recombinant GGPPS gene in order to provide increased levels of the diterpene precursor geranylgeranyl diphosphate, for increased flux through the rebaudioside A biosynthetic pathway. In some embodiments, the recombinant host further contains a construct to silence or reduce the expression of non-steviol pathways consuming geranylgeranyl diphosphate, ent-Kaurenoic acid or farnesyl pyrophosphate, thereby providing increased flux through the steviol and steviol glycosides biosynthetic pathways. For example, flux to sterol production pathways such as ergosterol may be reduced by downregulation of the ERG9 gene. In cells that produce gibberellins, gibberellin synthesis may be downregulated to increase flux of ent-kaurenoic acid to steviol. In carotenoid-producing organisms, flux to steviol may be increased by downregulation of one or more carotenoid biosynthetic genes.

In some embodiments, the recombinant host further contains and expresses recombinant genes involved in diterpene biosynthesis or production of terpenoid precursors, e.g., genes in the MEP or MEV pathway, have reduced phosphatase activity, and/or express a SUS.

In some embodiments, a recombinant host such as a microorganism produces steviol glycoside compositions that have greater than at least 15% rebaudioside C of the total steviol glycosides, e.g., at least 20% rebaudioside C, 30-40% rebaudioside C, 40-50% rebaudioside C, 50-60% rebaudioside C, 60-70% rebaudioside C, 70-80% rebaudioside C, 80-90% rebaudioside C. In some embodiments, a recombinant host such as a microorganism produces steviol glycoside compositions that have at least 90% rebaudioside C, e.g., 90-99% rebaudioside C. Other steviol glycosides present may include those depicted in FIGS. 2A and B such as steviol monosides, steviol glucobiosides, steviol rhamnobiosides, rebaudioside A, and Dulcoside A. In some embodiments, the rebaudioside C-enriched composition produced by the host can be further purified and the rebaudioside C or Dulcoside A so purified may then be mixed with other steviol glycosides, flavors, or sweeteners to obtain a desired flavor system or sweetening composition. For instance, a rebaudioside C-enriched composition produced by a recombinant microorganism can be combined with a rebaudioside A, F, or D-enriched composition produced by a different recombinant microorganism, with rebaudioside A, F, or D purified from a Stevia extract, or with rebaudioside A, F, or D produced in vitro.

### B. 4 Rebaudioside F Biosynthesis Polypeptides

The biosynthesis of rebaudioside F involves glucosylation and xylosylation of the aglycone steviol. Specifically, rebaudioside F can be formed by glucosylation of the 13-OH of steviol which forms steviol-13-O-glucoside, xylosylation of the C-2′ of the 13-O-glucose of steviol-13-O-glucoside which forms steviol-1,2-xylobioside, glucosylation of the C-19 carboxyl of the 1,2-xylobioside to form 1,2-stevioxyloside, and glucosylation of the C-3′ of the C-13-O-glucose of 1,2-stevioxyloside to form rebaudioside F. The order in which each glycosylation reaction occurs can vary. See FIG. 2D.

It has been discovered that conversion of steviol to rebaudioside F in a recombinant host can be accomplished by the expression of genes encoding the following functional UGTs: 85C2, EUGT11 and/or 91D2e, 74G1, and 76G1, along with endogenous or recombinantly expressed UDP-glucose dehydrogenase and UDP-glucuronic acid decarboxylase. Thus, a recombinant microorganism expressing these four or five UGTs along with endogenous or recombinant UDP-glucose dehydrogenase and UDP-glucuronic acid decarboxylase can make rebaudioside F when fed steviol in the medium. Alternatively, a recombinant microorganism expressing two functional UGTs, EUGT11 or 91D2e, and 76G1, can make rebaudioside F when fed rubusoside in the medium. As another alternative, a recombinant microorganism expressing a functional UGT 76G1 can make rebaudioside F when fed 1,2 steviorhamnoside. As another alternative, a recombinant microorganism expressing 74G1, EUGT11 and/or 91D2e, 76G1, and can make rebaudioside F when fed the monoside, steviol-13-O-glucoside, in the medium. Similarly, conversion of steviol-19-O-glucoside to rebaudioside F in a recombinant microorganism can be accomplished by the expression of genes encoding UGTs 85C2, EUGT11 and/or 91D2e, and 76G1, when fed steviol-19-O-glucoside. Typically, one or more of these genes are recombinant genes that have been transformed into a host that does not naturally possess them.

Suitable EUGT11, UGT91D2, UGT74G1, UGT76G1 and UGT85C2 polypeptides include the functional UGT polypeptides discussed herein. In some embodiments, a UGT79B3 polypeptide is substituted for a UGT91, as discussed above. UDP-glucose dehydrogenase and UDP-glucuronic acid decarboxylase provide increased amounts of the UDP-xylose donor for xylosylation of the steviol compound acceptor. Suitable UDP-glucose dehydrogenases and UDP-glucuronic acid decarboxylases include those made by Arabidopsis thaliana or Cryptococcus neoformans. For example, suitable UDP-glucose dehydrogenase and UDP-glucuronic acid decarboxylases polypeptides can be encoded by the A. thaliana UGD1 gene and UXS3 gene, respectively. See, Oka and Jigami, FEBS J. 273: 2645-2657 (2006).

In some embodiments rebaudioside F can be produced using in vitro methods while supplying the appropriate UDP-sugar and/or a cell-free system for regeneration of UDP-sugars. See, for example, Jewett et al. Molecular Systems Biology, Vol. 4, article 220 (2008); Masada et al. FEBS Lett. 581: 2562-2566 (2007). In some embodiments, sucrose and a sucrose synthase are provided in the reaction vessel in order to regenerate UDP-glucose from UDP during the glycosylation reactions. The sucrose synthase can be from any suitable organism. For example, a sucrose synthase coding sequence from Arabidopsis thaliana, Stevia rebaudiana, or Coffea arabica can be cloned into an expression plasmid under control of a suitable promoter, and expressed in a microorganism. In some embodiments, UDP-xylose can be produced from UDP-glucose by supplying suitable enzymes, for example, the Arabidopsis thaliana UGD1 (UDP-glucose dehydrogenase) and UXS3 (UDP-glucuronic acid decarboxylase) enzymes along with NAD+ cofactor.

Reactions may be carried out together, or stepwise. For instance, rebaudioside F may be produced from rubusoside with the addition of stoichiometric amounts of UDP-xylose and EUGT11, followed by addition of UGT76G1 and an excess or stoichiometric supply of UDP-glucose. In some embodiments, phosphatases are used to remove secondary products and improve the reaction yields. UGTs and other enzymes for in vitro reactions may be provided in soluble forms or immobilized forms. In some embodiments, rebaudioside F or other steviol xylosides can be produced using whole cells as discussed above. For example, the cells may contain UGT 76G1 and EUGT11 such that mixtures of stevioside and RebA are efficiently converted to RebD. In some embodiments, the whole cells are the host cells described in section III A.

In other embodiments, the recombinant host expresses one or more genes involved in steviol biosynthesis, e.g., a CDPS gene, a KS gene, a KO gene and/or a KAH gene. Thus, for example, a microorganism containing a CDPS gene, a KS gene, a KO gene and a KAH gene, in addition to a EUGT11, UGT85C2, a UGT74G1, an optional UGT91D2 gene, and a UGT76G1 gene, is capable of producing rebaudioside F without the necessity for including steviol in the culture media. In addition, the recombinant host typically expresses an endogenous or a recombinant gene encoding a UDP-glucose dehydrogenase and a UDP-glucuronic acid decarboxylase. Such genes are useful in order to provide increased amounts of the UDP-xylose donor for xylosylation of the steviol compound acceptor. Suitable UDP-glucose dehydrogenases and UDP-glucuronic acid decarboxylases include those made by Arabidopsis thaliana or Cryptococcus neoformans. For example, suitable UDP-glucose dehydrogenase and UDP-glucuronic acid decarboxylases polypeptides can be encoded by the A. thaliana UGD1 gene and UXS3 gene, respectively. See, Oka and Jigami FEBS J. 273:2645-2657 (2006).

One with skill in the art will recognize that by modulating relative expression levels of different UGT genes as well as modulating the availability of UDP-xylose, a recombinant microorganism can be tailored to specifically produce steviol and steviol glycoside products in a desired proportion. Transcriptional regulation of steviol biosynthesis genes can be achieved by a combination of transcriptional activation and repression using techniques known to those in the art. For in vitro reactions, one with skill in the art will recognize that addition of different levels of UGT enzymes in combination or under conditions which impact the relative activities of the different UGTS in combination will direct synthesis towards a desired proportion of each steviol glycosides.

In some embodiments, the recombinant host further contains and expresses a recombinant GGPPS gene in order to provide increased levels of the diterpene precursor geranylgeranyl diphosphate, for increased flux through the steviol biosynthetic pathway. In some embodiments, the recombinant host further contains a construct to silence the expression of non-steviol pathways consuming geranylgeranyl diphosphate, ent-Kaurenoic acid or farnesyl pyrophosphate, thereby providing increased flux through the steviol and steviol glycosides biosynthetic pathways. For example, flux to sterol production pathways such as ergosterol may be reduced by downregulation of the ERG9 gene. See, the ERG9 section below and Examples 24-25. In cells that produce gibberellins, gibberellin synthesis may be downregulated to increase flux of ent-kaurenoic acid to steviol. In carotenoid-producing organisms, flux to steviol may be increased by downregulation of one or more carotenoid biosynthetic genes. In some embodiments, the recombinant host further contains and expresses recombinant genes involved in diterpene biosynthesis, e.g., genes in the MEP pathway discussed below.

In some embodiments, a recombinant host such as a microorganism produces rebaudioside F-enriched steviol glycoside compositions that have greater than at least 4% rebaudioside F by weight total steviol glycosides, e.g., at least 5% rebaudioside F, at least 6% of rebaudioside F, 10-20% rebaudioside F, 20-30% rebaudioside F, 30-40% rebaudioside F, 40-50% rebaudioside F, 50-60% rebaudioside F, 60-70% rebaudioside F, 70-80% rebaudioside F. In some embodiments, a recombinant host such as a microorganism produces steviol glycoside compositions that have at least 90% rebaudioside F, e.g., 90-99% rebaudioside F. Other steviol glycosides present may include those depicted in FIGS. 2A and D such as steviol monosides, steviol glucobiosides, steviol xylobiosides, rebaudioside A, stevioxyloside, rubusoside and stevioside. In some embodiments, the rebaudioside F-enriched composition produced by the host can be mixed with other steviol glycosides, flavors, or sweeteners to obtain a desired flavor system or sweetening composition. For instance, a rebaudioside F-enriched composition produced by a recombinant microorganism can be combined with a rebaudioside A, C, or D-enriched composition produced by a different recombinant microorganism, with rebaudioside A, C, or D purified from a Stevia extract, or with rebaudioside A, C, or D produced in vitro.

### C. Other Polypeptides

Genes for additional polypeptides whose expression facilitates more efficient or larger scale production of steviol or a steviol glycoside can also be introduced into a recombinant host. For example, a recombinant microorganism can also contain one or more genes encoding a geranylgeranyl diphosphate synthase (GGPPS, also referred to as GGDPS). As another example, the recombinant host can contain one or more genes encoding a rhamnose synthetase, or one or more genes encoding a UDP-glucose dehydrogenase and/or a UDP-glucuronic acid decarboxylase. As another example, a recombinant host can also contain one or more genes encoding a cytochrome P450 reductase (CPR). Expression of a recombinant CPR facilitates the cycling of NADP+ to regenerate NADPH, which is utilized as a cofactor for terpenoid biosynthesis. Other methods can be used to regenerate NADHP levels as well. In circumstances where NADPH becomes limiting; strains can be further modified to include exogenous transhydrogenase genes. See, e.g., Sauer et al. J. Biol. Chem. 279: 6613-6619 (2004). Other methods are known to those with skill in the art to reduce or otherwise modify the ratio of NADH/NADPH such that the desired cofactor level is increased.

As another example, the recombinant host can contain one or more genes encoding one or more enzymes in the MEP pathway or the mevalonate pathway. Such genes are useful because they can increase the flux of carbon into the diterpene biosynthesis pathway, producing geranylgeranyl diphosphate from isopentenyl diphosphate and dimethylallyl diphosphate generated by the pathway. The geranylgeranyl diphosphate so produced can be directed towards steviol and steviol glycoside biosynthesis due to expression of steviol biosynthesis polypeptides and steviol glycoside biosynthesis polypeptides.

As another example the recombinant host can contain one or more genes encoding a sucrose synthase, and additionally can contain sucrose uptake genes if desired. The sucrose synthase reaction can be used to increase the UDP-glucose pool in a fermentation host, or in a whole cell bioconversion process. This regenerates UDP-glucose from UDP produced during glycosylation and sucrose, allowing for efficient glycosylation. In some organisms, disruption of the endogenous invertase is advantageous to prevent degradation of sucrose. For example, the S. cerevisiae SUC2 invertase may be disrupted. The sucrose synthase (SUS) can be from any suitable organism. For example, a sucrose synthase coding sequence from, without limitation, Arabidopsis thaliana, Stevia rebaudiana, or Coffea arabica can be cloned into an expression plasmid under control of a suitable promoter, and expressed in a microorganism. The sucrose synthase can be expressed in such a strain in combination with a sucrose transporter (e.g., the A. thaliana SUC1 transporter or a functional homolog thereof) and one or more UGTs (e.g., one or more of UGT85C2, UGT74G1, UGT76G1, and UGT91 D2e, EUGT11 or functional homologs thereof). Culturing the host in a medium that contains sucrose can promote production of UDP-glucose, as well as one or more glucosides (e.g., steviol glycosides).

In addition, a recombinant host can have reduced phosphatase activity as discussed herein.

### D. Functional Homologs

Functional homologs of the polypeptides described above are also suitable for use in producing steviol or steviol glycosides in a recombinant host. A functional homolog is a polypeptide that has sequence similarity to a reference polypeptide, and that carries out one or more of the biochemical or physiological function(s) of the reference polypeptide. A functional homolog and the reference polypeptide may be natural occurring polypeptides, and the sequence similarity may be due to convergent or divergent evolutionary events. As such, functional homologs are sometimes designated in the literature as homologs, or orthologs, or paralogs. Variants of a naturally occurring functional homolog, such as polypeptides encoded by mutants of a wild type coding sequence, may themselves be functional homologs. Functional homologs can also be created via site-directed mutagenesis of the coding sequence for a polypeptide, or by combining domains from the coding sequences for different naturally-occurring polypeptides (“domain swapping”). Techniques for modifying genes encoding functional UGT polypeptides described herein are known and include, inter alia, directed evolution techniques, site-directed mutagenesis techniques and random mutagenesis techniques, and can be useful to increase specific activity of a polypeptide, alter substrate specificity, alter expression levels, alter subcellular location, or modify polypeptide:polypeptide interactions in a desired manner. Such modified polypeptides are considered functional homologs. The term “functional homolog” is sometimes applied to the nucleic acid that encodes a functionally homologous polypeptide.

Functional homologs can be identified by analysis of nucleotide and polypeptide sequence alignments. For example, performing a query on a database of nucleotide or polypeptide sequences can identify homologs of steviol or steviol glycoside biosynthesis polypeptides. Sequence analysis can involve BLAST, Reciprocal BLAST, or PSI-BLAST analysis of nonredundant databases using a GGPPS, a CDPS, a KS, a KO or a KAH amino acid sequence as the reference sequence. Amino acid sequence is, in some instances, deduced from the nucleotide sequence. Those polypeptides in the database that have greater than 40% sequence identity are candidates for further evaluation for suitability as a steviol or steviol glycoside biosynthesis polypeptide. Amino acid sequence similarity allows for conservative amino acid substitutions, such as substitution of one hydrophobic residue for another or substitution of one polar residue for another. If desired, manual inspection of such candidates can be carried out in order to narrow the number of candidates to be further evaluated. Manual inspection can be performed by selecting those candidates that appear to have domains present in steviol biosynthesis polypeptides, e.g., conserved functional domains.

Conserved regions can be identified by locating a region within the primary amino acid sequence of a steviol or a steviol glycoside biosynthesis polypeptide that is a repeated sequence, forms some secondary structure (e.g., helices and beta sheets), establishes positively or negatively charged domains, or represents a protein motif or domain. See, e.g., the Pfam web site describing consensus sequences for a variety of protein motifs and domains on the World Wide Web at sanger.ac.uk/Software/Pfam/ and pfam.janelia.org/. The information included at the Pfam database is described in Sonnhammer et al. Nucl. Acids Res. 26: 320-322 (1998); Sonnhammer et al. Proteins 28: 405-420 (1997); and Bateman et al. Nucl. Acids Res. 27: 260-262 (1999). Conserved regions also can be determined by aligning sequences of the same or related polypeptides from closely related species. Closely related species preferably are from the same family. In some embodiments, alignment of sequences from two different species is adequate.

Typically, polypeptides that exhibit at least about 40% amino acid sequence identity are useful to identify conserved regions. Conserved regions of related polypeptides exhibit at least 45% amino acid sequence identity (e.g., at least 50%, at least 60%, at least 70%, at least 80%, or at least 90% amino acid sequence identity). In some embodiments, a conserved region exhibits at least 92%, 94%, 96%, 98%, or 99% amino acid sequence identity.

For example, polypeptides suitable for producing steviol glycosides in a recombinant host include functional homologs of EUGT11, UGT91D2e, UGT91D2m, UGT85C, and UGT76G. Such homologs have greater than 90% (e.g., at least 95% or 99%) sequence identity to the amino acid sequence of EUGT11, UGT91D2e, UGT91D2m, UGT85C, or UGT76G as set forth in PCT Application No. PCT/US2012/050021. Variants of EUGT11, UGT91D2, UGT85C, and UGT76G polypeptides typically have 10 or fewer amino acid substitutions within the primary amino acid sequence, e.g., 7 or fewer amino acid substitutions, 5 or conservative amino acid substitutions, or between 1 and 5 substitutions. However, in some embodiments, variants of EUGT11, UGT91D2, UGT85C, and UGT76G polypeptides can have 10 or more amino acid substitutions (e.g., 10, 15, 20, 25, 30, 35, 10-20, 10-35, 20-30, or 25-35 amino acid substitutions). The substitutions may be conservative, or in some embodiments, non-conservative. Non-limiting examples of non-conservative changes in UGT91D2e polypeptides include glycine to arginine and tryptophan to arginine. Non-limiting examples of non-conservative substitutions in UGT76G polypeptides include valine to glutamic acid, glycine to glutamic acid, glutamine to alanine, and serine to proline. Non-limiting examples of changes to UGT85C polypeptides include histidine to aspartic acid, proline to serine, lysine to threonine, and threonine to arginine.

In some embodiments, a useful UGT91D2 homolog can have amino acid substitutions (e.g., conservative amino acid substitutions) in regions of the polypeptide that are outside of predicted loops, e.g., residues 20-26, 39-43, 88-95, 121-124, 142-158, 185-198, and 203-214 are predicted loops in the N-terminal domain and residues 381-386 are predicted loops in the C-terminal domain of SEQ ID NO:5 as set forth in PCT Application No. PCT/US2012/050021. For example, a useful UGT91D2 homolog can include at least one amino acid substitution at residues 1-19, 27-38, 44-87, 96-120, 125-141, 159-184, 199-202, 215-380, or 387-473. In some embodiments, a UGT91D2 homolog can have an amino acid substitution at one or more residues selected from the group consisting of residues 30, 93, 99, 122, 140, 142, 148, 153, 156, 195, 196, 199, 206, 207, 211, 221, 286, 343, 427, and 438. For example, a UGT91 D2 functional homolog can have an amino acid substitution at one or more of residues 206, 207, and 343, such as an arginine at residue 206, a cysteine at residue 207, and an arginine at residue 343. See, SEQ ID NO:95 of PCT Application No. PCT/US2012/050021. Other functional homologs of UGT91D2 can have one or more of the following: a tyrosine or phenylalanine at residue 30, a proline or glutamine at residue 93, a serine or valine at residue 99, a tyrosine or a phenylalanine at residue 122, a histidine or tyrosine at residue 140, a serine or cysteine at residue 142, an alanine or threonine at residue 148, a methionine at residue 152, an alanine at residue 153, an alanine or serine at residue 156, a glycine at residue 162, a leucine or methionine at residue 195, a glutamic acid at residue 196, a lysine or glutamic acid at residue 199, a leucine or methionine at residue 211, a leucine at residue 213, a serine or phenylalanine at residue 221, a valine or isoleucine at residue 253, a valine or alanine at residue 286, a lysine or asparagine at residue 427, an alanine at residue 438, and either an alanine or threonine at residue 462. In another embodiment, a UGT91 D2 functional homolog contains a methionine at residue 211 and an alanine at residue 286.

In some embodiments, a useful UGT85C homolog can have one or more amino acid substitutions at residues 9, 10, 13, 15, 21, 27, 60, 65, 71, 87, 91, 220, 243, 270, 289, 298, 334, 336, 350, 368, 389, 394, 397, 418, 420, 440, 441, 444, and 471 of SEQ ID NO:3 as set forth in PCT Application No. PCT/US2012/050021. Non-limiting examples of useful UGT85C homologs include polypeptides having substitutions at residue 65 (e.g., a serine at residue 65), at residue 65 in combination with residue 15 (a leucine at residue 15), 270 (e.g., a methionine, arginine, or alanine at residue 270), 418 (e.g., a valine at residue 418), 440 (e.g., an aspartic acid at residue at residue 440), or 441 (e.g., an asparagine at residue 441); residues 13 (e.g., a phenylalanine at residue 13), 15, 60 (e.g., an aspartic acid at residue 60), 270, 289 (e.g., a histidine at residue 289), and 418; substitutions at residues 13, 60, and 270; substitutions at residues 60 and 87 (e.g., a phenylalanine at residue 87); substitutions at residues 65, 71 (e.g., a glutamine at residue 71), 220 (e.g., a threonine at residue 220), 243 (e.g., a tryptophan at residue 243), and 270; substitutions at residues 65, 71, 220, 243, 270, and 441; substitutions at residues 65, 71, 220, 389 (e.g., a valine at residue 389), and 394 (e.g., a valine at residue 394); substitutions at residues 65, 71, 270, and 289; substitutions at residues 220, 243, 270, and 334 (e.g., a serine at residue 334); or substitutions at residues 270 and 289. The following amino acid mutations did not result in a loss of activity in 85C2 polypeptides: V13F, F15L, H60D, A65S, E71Q, I87F, K220T, R243W, T270M, T270R, Q289H, L334S, A389V, I394V, P397S, E418V, G440D, and H441N. Additional mutations that were seen in active clones include K9E, K10R, Q21H, M27V, L91P, Y298C, K350T, H368R, G420R, L431P, R444G, and M471T. In some embodiments, an UGT85C2 contains substitutions at positions 65 (e.g., a serine), 71 (a glutamine), 270 (a methionine), 289 (a histidine), and 389 (a valine).

The amino acid sequence of Stevia rebaudiana UGTs 74G1,76G1 and 91D2e with N-terminal, in-frame fusions of the first 158 amino acids of human MDM2 protein, and Stevia rebaudiana UGT85C2 with an N-terminal in-frame fusion of 4 repeats of the synthetic PMI peptide (4 X TSFAEYWNLLSP, SEQ ID NO:1) as set forth in SEQ ID NOs: 90, 88, 94, and 92 of PCT Application No. PCT/US2012/050021.

In some embodiments, a useful UGT76G homolog can have one or more amino acid substitutions at residues 29, 74, 87, 91, 116, 123, 125, 126, 130, 145, 192, 193, 194, 196, 198, 199, 200, 203, 204, 205, 206, 207, 208, 266, 273, 274, 284, 285, 291, 330, 331, and 346 of SEQ ID NO:7 as set forth in PCT Application No. PCT/US2012/050021. Non-limiting examples of useful UGT76G homologs include polypeptides having substitutions at residues 74, 87, 91, 116, 123, 125, 126, 130, 145, 192, 193, 194, 196, 198, 199, 200, 203, 204, 205, 206, 207, 208, and 291; residues 74, 87, 91, 116, 123, 125, 126, 130, 145, 192, 193, 194, 196, 198, 199, 200, 203, 204, 205, 206, 207, 208, 266, 273, 274, 284, 285, and 291; or residues 74, 87, 91, 116, 123, 125, 126, 130, 145, 192, 193, 194, 196, 198, 199, 200, 203, 204, 205, 206, 207, 208, 266, 273, 274, 284, 285, 291, 330, 331, and 346. See, Table 9.

TABLE 9

| Clone | Mutations |
|---|---|
| 76G_G7 | M29I, V74E, V87G, L91P, G116E, A123T, Q125A, I126L, |
| | T130A, V145M, C192S, S193A, F194Y, M196N, K198Q, |
| | K199I, Y200L, Y203I, F204L, E205G, N206K, I207M, T208I, |
| | P266Q, S273P, R274S, G284T, T285S, 287-3 by deletion, |
| | L330V, G331A, L346I |
| 76G_H12 | M29I, V74E, V87G, L91P, G116E, A123T, Q125A, I126L, |
| | T130A, V145M, C192S, S193A, F194Y, M196N, K198Q, |
| | K1991, Y200L, Y203I, F204L, E205G, N206K, I207M, T208I, |
| | P266Q, S273P, R274S, G284T, T285S, 287-3 by deletion |
| 76G_C4 | M29I, V74E, V87G, L91P, G116E, A123T, Q125A, I126L, |
| | T130A, V145M, C192S, S193A, F194Y, M196N, K198Q, |
| | K199I, Y200L, Y203I, F204L, E205G, N206K, I207M, T208I |

Methods to modify the substrate specificity of, for example, EUGT11 or UGT91D2e, are known to those skilled in the art, and include without limitation site-directed/rational mutagenesis approaches, random directed evolution approaches and combinations in which random mutagenesis/saturation techniques are performed near the active site of the enzyme. For example see Osmani et al. Phytochemistry 70: 325-347 (2009).

A candidate sequence typically has a length that is from 80 percent to 200 percent of the length of the reference sequence, e.g., 82, 85, 87, 89, 90, 93, 95, 97, 99, 100, 105, 110, 115, 120, 130, 140, 150, 160, 170, 180, 190, or 200 percent of the length of the reference sequence. A functional homolog polypeptide typically has a length that is from 95 percent to 105 percent of the length of the reference sequence, e.g., 90, 93, 95, 97, 99, 100, 105, 110, 115, or 120 percent of the length of the reference sequence, or any range between. A percent identity for any candidate nucleic acid or polypeptide relative to a reference nucleic acid or polypeptide can be determined as follows. A reference sequence (e.g., a nucleic acid sequence or an amino acid sequence) is aligned to one or more candidate sequences using the computer program ClustalW (version 1.83, default parameters), which allows alignments of nucleic acid or polypeptide sequences to be carried out across their entire length (global alignment). Chenna et al., Nucleic Acids Res., 31(13):3497-500 (2003).

ClustalW calculates the best match between a reference and one or more candidate sequences, and aligns them so that identities, similarities and differences can be determined. Gaps of one or more residues can be inserted into a reference sequence, a candidate sequence, or both, to maximize sequence alignments. For fast pairwise alignment of nucleic acid sequences, the following default parameters are used: word size: 2; window size: 4; scoring method: percentage; number of top diagonals: 4; and gap penalty: 5. For multiple alignment of nucleic acid sequences, the following parameters are used: gap opening penalty: 10.0; gap extension penalty: 5.0; and weight transitions: yes. For fast pairwise alignment of protein sequences, the following parameters are used: word size: 1; window size: 5; scoring method: percentage; number of top diagonals: 5; gap penalty: 3. For multiple alignment of protein sequences, the following parameters are used: weight matrix: blosum; gap opening penalty: 10.0; gap extension penalty: 0.05; hydrophilic gaps: on; hydrophilic residues: Gly, Pro, Ser, Asn, Asp, Gln, Glu, Arg, and Lys; residue-specific gap penalties: on. The ClustalW output is a sequence alignment that reflects the relationship between sequences. ClustalW can be run, for example, at the Baylor College of Medicine Search Launcher site on the World Wide Web (searchlauncher.bcm.tmc.edu/multi-align/multi-align.html) and at the European Bioinformatics Institute site on the World Wide Web (ebi.ac.uk/clustalw).

To determine percent identity of a candidate nucleic acid or amino acid sequence to a reference sequence, the sequences are aligned using ClustalW, the number of identical matches in the alignment is divided by the length of the reference sequence, and the result is multiplied by 100. It is noted that the percent identity value can be rounded to the nearest tenth. For example, 78.11, 78.12, 78.13, and 78.14 are rounded down to 78.1, while 78.15, 78.16, 78.17, 78.18, and 78.19 are rounded up to 78.2.

It will be appreciated that functional UGTs can include additional amino acids that are not involved in glucosylation or other enzymatic activities carried out by the enzyme, and thus such a polypeptide can be longer than would otherwise be the case. For example, a EUGT11 polypeptide can include a purification tag (e.g., HIS tag or GST tag), a chloroplast transit peptide, a mitochondrial transit peptide, an amyloplast peptide, signal peptide, or a secretion tag added to the amino or carboxy terminus. In some embodiments, a EUGT11 polypeptide includes an amino acid sequence that functions as a reporter, e.g., a green fluorescent protein or yellow fluorescent protein.

### II. Steviol and Steviol Glycoside Biosynthesis Nucleic Acids

A recombinant gene encoding a polypeptide described herein comprises the coding sequence for that polypeptide, operably linked in sense orientation to one or more regulatory regions suitable for expressing the polypeptide. Because many microorganisms are capable of expressing multiple gene products from a polycistronic mRNA, multiple polypeptides can be expressed under the control of a single regulatory region for those microorganisms, if desired. A coding sequence and a regulatory region are considered to be operably linked when the regulatory region and coding sequence are positioned so that the regulatory region is effective for regulating transcription or translation of the sequence. Typically, the translation initiation site of the translational reading frame of the coding sequence is positioned between one and about fifty nucleotides downstream of the regulatory region for a monocistronic gene.

In many cases, the coding sequence for a polypeptide described herein is identified in a species other than the recombinant host, i.e., is a heterologous nucleic acid. Thus, if the recombinant host is a microorganism, the coding sequence can be from other prokaryotic or eukaryotic microorganisms, from plants or from animals. In some case, however, the coding sequence is a sequence that is native to the host and is being reintroduced into that organism. A native sequence can often be distinguished from the naturally occurring sequence by the presence of non-natural sequences linked to the exogenous nucleic acid, e.g., non-native regulatory sequences flanking a native sequence in a recombinant nucleic acid construct. In addition, stably transformed exogenous nucleic acids typically are integrated at positions other than the position where the native sequence is found.

“Regulatory region” refers to a nucleic acid having nucleotide sequences that influence transcription or translation initiation and rate, and stability and/or mobility of a transcription or translation product. Regulatory regions include, without limitation, promoter sequences, enhancer sequences, response elements, protein recognition sites, inducible elements, protein binding sequences, 5′ and 3′ untranslated regions (UTRs), transcriptional start sites, termination sequences, polyadenylation sequences, introns, and combinations thereof. A regulatory region typically comprises at least a core (basal) promoter. A regulatory region also may include at least one control element, such as an enhancer sequence, an upstream element or an upstream activation region (UAR). A regulatory region is operably linked to a coding sequence by positioning the regulatory region and the coding sequence so that the regulatory region is effective for regulating transcription or translation of the sequence. For example, to operably link a coding sequence and a promoter sequence, the translation initiation site of the translational reading frame of the coding sequence is typically positioned between one and about fifty nucleotides downstream of the promoter. A regulatory region can, however, be positioned as much as about 5,000 nucleotides upstream of the translation initiation site, or about 2,000 nucleotides upstream of the transcription start site.

The choice of regulatory regions to be included depends upon several factors, including, but not limited to, efficiency, selectability, inducibility, desired expression level, and preferential expression during certain culture stages. It is a routine matter for one of skill in the art to modulate the expression of a coding sequence by appropriately selecting and positioning regulatory regions relative to the coding sequence. It will be understood that more than one regulatory region may be present, e.g., introns, enhancers, upstream activation regions, transcription terminators, and inducible elements.

One or more genes can be combined in a recombinant nucleic acid construct in “modules” useful for a discrete aspect of steviol and/or steviol glycoside production. Combining a plurality of genes in a module, particularly a polycistronic module, facilitates the use of the module in a variety of species. For example, a steviol biosynthesis gene cluster, or a UGT gene cluster, can be combined in a polycistronic module such that, after insertion of a suitable regulatory region, the module can be introduced into a wide variety of species. As another example, a UGT gene cluster can be combined such that each UGT coding sequence is operably linked to a separate regulatory region, to form a UGT module. Such a module can be used in those species for which monocistronic expression is necessary or desirable. In addition to genes useful for steviol or steviol glycoside production, a recombinant construct typically also contains an origin of replication, and one or more selectable markers for maintenance of the construct in appropriate species.

It will be appreciated that because of the degeneracy of the genetic code, a number of nucleic acids can encode a particular polypeptide; i.e., for many amino acids, there is more than one nucleotide triplet that serves as the codon for the amino acid. Thus, codons in the coding sequence for a given polypeptide can be modified such that optimal expression in a particular host is obtained, using appropriate codon bias tables for that host (e.g., microorganism). SEQ ID NOs:18-25, 34-36, 40-43, 48-49, 52-55, 60-64, 70-72, and 154 of PCT Application No. PCT/US2012/050021 set forth nucleotide sequences encoding certain enzymes for steviol and steviol glycoside biosynthesis, modified for increased expression in yeast. As isolated nucleic acids, these modified sequences can exist as purified molecules and can be incorporated into a vector or a virus for use in constructing modules for recombinant nucleic acid constructs.

In some cases, it is desirable to inhibit one or more functions of an endogenous polypeptide in order to divert metabolic intermediates towards steviol or steviol glycoside biosynthesis. For example, it may be desirable to downregulate synthesis of sterols in a yeast strain in order to further increase steviol or steviol glycoside production, e.g., by downregulating squalene epoxidase. As another example, it may be desirable to inhibit degradative functions of certain endogenous gene products, e.g., glycohydrolases that remove glucose moieties from secondary metabolites or phosphatases as discussed herein. As another example, expression of membrane transporters involved in transport of steviol glycosides can be inhibited, such that secretion of glycosylated steviosides is inhibited. Such regulation can be beneficial in that secretion of steviol glycosides can be inhibited for a desired period of time during culture of the microorganism, thereby increasing the yield of glycoside product(s) at harvest. In such cases, a nucleic acid that inhibits expression of the polypeptide or gene product may be included in a recombinant construct that is transformed into the strain. Alternatively, mutagenesis can be used to generate mutants in genes for which it is desired to inhibit function.

### III. Hosts

A number of prokaryotes and eukaryotes are suitable for use in constructing the recombinant microorganisms described herein, e.g., gram-negative bacteria, yeast and fungi. A species and strain selected for use as a steviol or steviol glycoside production strain is first analyzed to determine which production genes are endogenous to the strain and which genes are not present. Genes for which an endogenous counterpart is not present in the strain are assembled in one or more recombinant constructs, which are then transformed into the strain in order to supply the missing function(s).

Exemplary prokaryotic and eukaryotic species are described in more detail below. However, it will be appreciated that other species may be suitable. For example, suitable species may be in a genus selected from the group consisting of Agaricus, Aspergillus, Bacillus, Candida, Corynebacterium, Escherichia, Fusarium/Gibberella, Kluyveromyces, Laetiporus, Lentinus, Phaffia, Phanerochaete, Pichia, Physcomitrella, Rhodoturula, Saccharomyces, Schizosaccharomyces, Sphaceloma, Xanthophyllomyces and Yarrowia. Exemplary species from such genera include Lentinus tigrinus, Laetiporus sulphureus, Phanerochaete chrysosporium, Pichia pastoris, Physcomitrella patens, Rhodoturula glutinis 32, Rhodoturula mucilaginosa, Phaffia rhodozyma UBV-AX, Xanthophyllomyces dendrorhous, Fusarium fujikuroilGibberella fujikuroi, Candida utilis and Yarrowia lipolytica. In some embodiments, a microorganism can be an Ascomycete such as Gibberella fujikuroi, Kluyveromyces lactis, Schizosaccharomyces pombe, Aspergillus niger, or Saccharomyces cerevisiae. In some embodiments, a microorganism can be a prokaryote such as Escherichia coli, Rhodobacter sphaeroides, or Rhodobacter capsulatus. It will be appreciated that certain microorganisms can be used to screen and test genes of interest in a high throughput manner, while other microorganisms with desired productivity or growth characteristics can be used for large-scale production of steviol glycosides.

Saccharomyces cerevisiae

Saccharomyces cerevisiae is a widely used chassis organism in synthetic biology, and can be used as the recombinant microorganism platform. There are libraries of mutants, plasmids, detailed computer models of metabolism and other information available for S. cerevisiae, allowing for rational design of various modules to enhance product yield. Methods are known for making recombinant microorganisms.

A steviol biosynthesis gene cluster can be expressed in yeast using any of a number of known promoters. Strains that overproduce terpenes are known and can be used to increase the amount of geranylgeranyl diphosphate available for steviol and steviol glycoside production.

Aspergillus Spp.

Aspergillus species such as A. oryzae, A. niger and A. sojae are widely used microorganisms in food production, and can also be used as the recombinant microorganism platform. Nucleotide sequences are available for genomes of A. nidulans, A. fumigatus, A. oryzae, A. clavatus, A. flavus, A. niger, and A. terreus, allowing rational design and modification of endogenous pathways to enhance flux and increase product yield. Metabolic models have been developed for Aspergillus, as well as transcriptomic studies and proteomics studies. A. niger is cultured for the industrial production of a number of food ingredients such as citric acid and gluconic acid, and thus species such as A. niger are generally suitable for the production of food ingredients such as steviol and steviol glycosides.

Escherichia coli

Escherichia coli, another widely used platform organism in synthetic biology, can also be used as the recombinant microorganism platform. Similar to Saccharomyces, there are libraries of mutants, plasmids, detailed computer models of metabolism and other information available for E. coli, allowing for rational design of various modules to enhance product yield. Methods similar to those described above for Saccharomyces can be used to make recombinant E. coli microorganisms.

Agaricus, Gibberella, and Phanerochaete Spp.

Agaricus, Gibberella, and Phanerochaete spp. can be useful because they are known to produce large amounts of gibberellin in culture. Thus, the terpene precursors for producing large amounts of steviol and steviol glycosides are already produced by endogenous genes. Thus, modules containing recombinant genes for steviol or steviol glycoside biosynthesis polypeptides can be introduced into species from such genera without the necessity of introducing mevalonate or MEP pathway genes.

Arxula adeninivorans (Blastobotrys adeninivorans)

Arxula adeninivorans is a dimorphic yeast (it grows as a budding yeast like the baker's yeast up to a temperature of 42° C., above this threshold it grows in a filamentous form) with unusual biochemical characteristics. It can grow on a wide range of substrates and can assimilate nitrate. It has successfully been applied to the generation of strains that can produce natural plastics or the development of a biosensor for estrogens in environmental samples.

Yarrowia lipolytica

Yarrowia lipolytica is a dimorphic yeast (see Arxula adeninivorans) that can grow on a wide range of substrates. It has a high potential for industrial applications but there are no recombinant products commercially available yet.

Rhodobacter Spp.

Rhodobacter can be use as the recombinant microorganism platform. Similar to E. coli, there are libraries of mutants available as well as suitable plasmid vectors, allowing for rational design of various modules to enhance product yield. Isoprenoid pathways have been engineered in membraneous bacterial species of Rhodobacter for increased production of carotenoid and CoQ10. See, U.S. Patent Publication Nos. 20050003474 and 20040078846. Methods similar to those described above for E. coli can be used to make recombinant Rhodobacter microorganisms.

Candida boidinii

Candida boidinii is a methylotrophic yeast (it can grow on methanol). Like other methylotrophic species such as Hansenula polymorpha and Pichia pastoris, it provides an excellent platform for the production of heterologous proteins. Yields in a multigram range of a secreted foreign protein have been reported. A computational method, IPRO, recently predicted mutations that experimentally switched the cofactor specificity of Candida boidinii xylose reductase from NADPH to NADH.

Hansenula polymorpha (Pichia angusta)

Hansenula polymorpha is another methylotrophic yeast (see Candida boidinii). It can furthermore grow on a wide range of other substrates; it is thermo-tolerant and can assimilate nitrate (see also Kluyveromyces lactis). It has been applied to the production of hepatitis B vaccines, insulin and interferon alpha-2a for the treatment of hepatitis C, furthermore to a range of technical enzymes.

Kluyveromyces lactis

Kluyveromyces lactis is a yeast regularly applied to the production of kefir. It can grow on several sugars, most importantly on lactose which is present in milk and whey. It has successfully been applied among others to the production of chymosin (an enzyme that is usually present in the stomach of calves) for the production of cheese. Production takes place in fermenters on a 40,000 L scale.

Pichia pastoris

Pichia pastoris is a methylotrophic yeast (see Candida boidinii and Hansenula polymorpha). It provides an efficient platform for the production of foreign proteins. Platform elements are available as a kit and it is worldwide used in academia for the production of proteins. Strains have been engineered that can produce complex human N-glycan (yeast glycans are similar but not identical to those found in humans).

Physcomitrella Spp.

Physcomitrella mosses, when grown in suspension culture, have characteristics similar to yeast or other fungal cultures. This genera is becoming an important type of cell for production of plant secondary metabolites, which can be difficult to produce in some other types of cells.

### IV. Methods of Producing Steviol Glycosides

Recombinant microorganisms described herein can be used in methods to produce steviol or steviol glycosides. For example, the method can include growing the recombinant microorganism in a culture medium under conditions in which steviol and/or steviol glycoside biosynthesis genes are expressed. The recombinant microorganism may be grown in a fed batch or continuous process. Typically, the recombinant microorganism is grown in a fermentor at a defined temperature(s) for a desired period of time. Depending on the particular microorganism used in the method, other recombinant genes such as isopentenyl biosynthesis genes and terpene synthase and cyclase genes may also be present and expressed. Levels of substrates and intermediates, e.g., isopentenyl diphosphate, dimethylallyl diphosphate, geranylgeranyl diphosphate, kaurene and kaurenoic acid, can be determined by extracting samples from culture media for analysis according to published methods.

After the recombinant microorganism has been grown in culture for the desired period of time, steviol and/or one or more steviol glycosides can then be recovered from the culture using various techniques known in the art. In some embodiments, a permeabilizing agent can be added to aid the feedstock entering into the host and product getting out. For example, a crude lysate of the cultured microorganism can be centrifuged to obtain a supernatant. The resulting supernatant can then be applied to a chromatography column, e.g., a C-18 column, and washed with water to remove hydrophilic compounds, followed by elution of the compound(s) of interest with a solvent such as methanol. The compound(s) can then be further purified by preparative HPLC. See also WO 2009/140394.

The amount of steviol glycoside (e.g., rebaudioside A or rebaudioside D) produced can be from about 1 mg/L to about 2000 mg/L, e.g., about 1 to about 10 mg/L, about 3 to about 10 mg/L, about 5 to about 20 mg/L, about 10 to about 50 mg/L, about 10 to about 100 mg/L, about 25 to about 500 mg/L, about 100 to about 1,500 mg/L, or about 200 to about 1,000 mg/L, at least about 1,000 mg/L, at least about 1,200 mg/L, at least about at least 1,400 mg/L, at least about 1,600 mg/L, at least about 1,800 mg/L, or at least about 2,000 mg/L. In general, longer culture times will lead to greater amounts of product. Thus, the recombinant microorganism can be cultured for from 1 day to 7 days, from 1 day to 5 days, from 3 days to 5 days, about 3 days, about 4 days, or about 5 days.

It will be appreciated that the various genes and modules discussed herein can be present in two or more recombinant microorganisms rather than a single microorganism. When a plurality of recombinant microorganisms is used, they can be grown in a mixed culture to produce steviol and/or steviol glycosides. For example, a first microorganism can comprise one or more biosynthesis genes for producing steviol while a second microorganism comprises steviol glycoside biosynthesis genes. Alternatively, the two or more microorganisms each can be grown in a separate culture medium and the product of the first culture medium, e.g., steviol, can be introduced into second culture medium to be converted into a subsequent intermediate, or into an end product such as rebaudioside A. The product produced by the second, or final microorganism is then recovered. It will also be appreciated that in some embodiments, a recombinant microorganism is grown using nutrient sources other than a culture medium and utilizing a system other than a fermentor.

Steviol glycosides do not necessarily have equivalent performance in different food systems. It is therefore desirable to have the ability to direct the synthesis to steviol glycoside compositions of choice. Recombinant hosts described herein can produce compositions that are selectively enriched for specific steviol glycosides (e.g., rebaudioside D) and have a consistent taste profile. Thus, the recombinant microorganisms described herein can facilitate the production of compositions that are tailored to meet the sweetening profile desired for a given food product and that have a proportion of each steviol glycoside that is consistent from batch to batch. Microorganisms described herein do not produce the undesired plant byproducts found in Stevia extracts. Thus, steviol glycoside compositions produced by the recombinant microorganisms described herein are distinguishable from compositions derived from Stevia plants.

Additional steps can be taken to obtain purified steviol glycosides, and compositions that are selectively enriched for one or more steviol glycosides (e.g., rebaudioside A, as described in the Examples below). For example, various adsorption steps, chromatography steps, crystallization steps, or combinations thereof can be used to purify or enrich for a steviol glycoside. In some embodiments (e.g., as described below), an extract or culture supernatant can be subjected to chromatography using a large-pore polymeric resin such as, for example, HP-20L or Amberlite XAD. The steviol glycoside can be eluted from the column using a suitable solvent (e.g., methanol, ethanol, water, or methanol/water or ethanol/water solutions, such as 5%, 10%, 15%, 20%, 25%, 30%, 35%. 40%, 45%, 50%, 55%, 60%, 65%, 70%, 75%, 80%, 85%, 90%, or 95% methanol/water), and fractions can be analyzed using high pressure liquid chromatography (HPLC), for example. In some cases, the water can be removed using one or more evaporation steps. In some cases, the polymeric resin can be used as an adsorbent resin.

In some cases, an extract or supernatant can be subjected to a preliminary step such as de-fatting (e.g., with hexane) or removal of polyphenols prior to further purification. In other cases, de-fatting with a nonpolar solvent is not employed.

In addition or alternatively, enrichment, concentration, or purification can be achieved by adsorbing fractions onto a carrier material (e.g., a diatomaceous earth material such as CELITE®).

In addition or alternatively, enrichment or purification can be achieved using medium pressure liquid chromatography (MPLC) fractionation. Concentrated fractions can be subjected to MPLC using, for example, methanol, acetonitrile, methanol/water, or acetonitrile/water (e.g., 5%, 10%, 15%, 20%, 25%, 30%, 35%. 40%, 45%, 50%, 55%, 60%, 65%, 70%, 75%, 80%, 85%, 90%, or 95% methanol/water or acetonitrile/water) as an eluent. The stationary phase may be a reversed phase resin (e.g., POLYGOPREP® 60-50 RP-18). A silica resin or other reversed-phase resin also may be useful for such purification steps, with the proper solvent and gradients.

Other purification techniques that take advantage of the hydrophobicity of impurities as compared to the target molecule and are amenable to large-scale chromatography can be used. For example, orthogonal chromatography methods such as hydrophobic interaction liquid chromatography (HILIC) and chromatography using the custom polymeric resin Uni PMM50-Carb (Nano-Micro Technology Company) may be useful to further purify a steviol glycoside such as RebA. Further, other types of chromatography methods can be used as alternatives to fractionation chromatography, including batch chromatography (adsorb/desorb), and simulated moving bed chromatography where higher resolution is needed. Further, methods such as those disclosed in U.S. Publication No. 2011/0087011 may be useful.

Crystallization of pooled chromatographic fractions containing a target steviol glycoside can be performed if desired. Typically, pooled fractions are evaporated to remove chromatography solvent, redissolved in a suitable crystallization solvent, and evaporated to remove crystallization solvent and permit crystallization of the glycoside. Suitable crystallization solvents include ethanol, methanol and dioxane. In addition to evaporative crystallization, other crystallization techniques that can be used include temperature-based crystallization and anti-solvent based crystallization. In some cases, repeated crystallizations can be used to increase the purity of the target steviol glycoside with respect to impurities from the fermentation process.

The purity of chromatography fractions or crystallized products can be assessed using, for example, analytical chromatography, and/or NMR. Additional techniques to remove remaining impurities can be used if desired.

Those skilled in the art will recognize that other techniques are commonly employed in the food industry, for example, decolorization with activated charcoal or another decolorization adsorbent can be employed if desired.

### V. Steviol Glycosides, Compositions, and Food Products

Steviol glycosides and steviol glycoside compositions produced and purified as described herein have improved sensory profiles relative to Stevia-derived glycosides. For example, such glycosides can have a faster sweetness build (i.e., a shorter time to maximum sweetness intensity), have an immediate sweetness onset (i.e., immediate perception of sweetness), have less artificial sweetness, have a less bitter taste, or have a less acidic taste than the corresponding steviol glycoside extracted from a Stevia plant. Less artificial sweetness refers to the intensity of flavor that is associated with known artificial sweeteners. Bitter taste is assessed as the taste of caffeine, and can be scored as having no perception of bitterness to very intense bitterness. Acidic taste is assessed as the taste of citric acid, and can be scored as having no perception of acidity to very intense acidity. Such characteristics can be assessed using trained sensory panels as described in the Examples.

The flavor characteristics of steviol glycosides and steviol glycoside compositions can be evaluated by a sensory panel using techniques known in the art. There are also sensory testing facilities that offer various sensory evaluation services. For example, North Carolina State University Sensory Service Center, Ohio State University Sensory Science Group, the Sensory Laboratory at Oregon State University, Monell Chemical Senses Center in Philadelphia, The National Food Lab (Livermore, Calif.), or Sensory Dimensions (Reading, United Kingdom) can be used to evaluated the flavor characteristics of steviol glycosides and steviol glycoside compositions.

Steviol glycosides and compositions obtained by the methods disclosed herein can be used to make food products, dietary supplements and sweetener compositions. For example, substantially pure steviol or steviol glycoside such as rebaudioside A or rebaudioside D can be included in food products such as ice cream, carbonated beverages, fruit juices, yogurts, baked goods, chewing gums, hard and soft candies, and sauces. Substantially pure steviol or steviol glycoside can also be included in non-food products such as pharmaceutical products, medicinal products, dietary supplements and nutritional supplements. Substantially pure steviol or steviol glycosides may also be included in animal feed products for both the agriculture industry and the companion animal industry. Alternatively, a mixture of steviol and/or steviol glycosides can be made by culturing recombinant microorganisms separately, each producing a specific steviol or steviol glycoside, recovering the steviol or steviol glycoside in substantially pure form from each microorganism and then combining the compounds to obtain a mixture containing each compound in the desired proportion. The recombinant microorganisms, plants, and plant cells described herein permit more precise and consistent mixtures to be obtained compared to current Stevia products. In another alternative, a substantially pure steviol or steviol glycoside can be incorporated into a food product along with other sweeteners, e.g. saccharin, dextrose, sucrose, fructose, erythritol, aspartame, sucralose, monatin, or acesulfame potassium. The weight ratio of steviol or steviol glycoside relative to other sweeteners can be varied as desired to achieve a satisfactory taste in the final food product. See, e.g., U.S. Patent Publication No. 2007/0128311. In some embodiments, the steviol or steviol glycoside may be provided with a flavor (e.g., citrus) as a flavor modulator. For example, Rebaudioside C can be used as a sweetness enhancer or sweetness modulator, in particular for carbohydrate based sweeteners, such that the amount of sugar can be reduced in the food product.

Compositions produced by a recombinant microorganism described herein can be incorporated into food products. For example, a steviol glycoside composition produced by a recombinant microorganism can be incorporated into a food product in an amount ranging from about 20 mg steviol glycoside/kg food product to about 1800 mg steviol glycoside/kg food product on a dry weight basis, depending on the type of steviol glycoside and food product. For example, a steviol glycoside composition produced by a recombinant microorganism can be incorporated into a dessert, cold confectionary (e.g., ice cream), dairy product (e.g., yogurt), or beverage (e.g., a carbonated beverage) such that the food product has a maximum of 500 mg steviol glycoside/kg food on a dry weight basis. A steviol glycoside composition produced by a recombinant microorganism can be incorporated into a baked good (e.g., a biscuit) such that the food product has a maximum of 300 mg steviol glycoside/kg food on a dry weight basis. A steviol glycoside composition produced by a recombinant microorganism can be incorporated into a sauce (e.g., chocolate syrup) or vegetable product (e.g., pickles) such that the food product has a maximum of 1000 mg steviol glycoside/kg food on a dry weight basis. A steviol glycoside composition produced by a recombinant microorganism can be incorporated into a bread such that the food product has a maximum of 160 mg steviol glycoside/kg food on a dry weight basis. A steviol glycoside composition produced by a recombinant microorganism, plant, or plant cell can be incorporated into a hard or soft candy such that the food product has a maximum of 1600 mg steviol glycoside/kg food on a dry weight basis. A steviol glycoside composition produced by a recombinant microorganism, plant, or plant cell can be incorporated into a processed fruit product (e.g., fruit juices, fruit filling, jams, and jellies) such that the food product has a maximum of 1000 mg steviol glycoside/kg food on a dry weight basis.

For example, such a steviol glycoside composition can have from 90-99% rebaudioside A and an undetectable amount of stevia plant-derived contaminants, and be incorporated into a food product at from 25-1600 mg/kg, e.g., 100-500 mg/kg, 25-100 mg/kg, 250-1000 mg/kg, 50-500 mg/kg or 500-1000 mg/kg on a dry weight basis.

Such a steviol glycoside composition can be a rebaudioside B-enriched composition having greater than 3% rebaudioside B and be incorporated into the food product such that the amount of rebaudioside B in the product is from 25-1600 mg/kg, e.g., 100-500 mg/kg, 25-100 mg/kg, 250-1000 mg/kg, 50-500 mg/kg or 500-1000 mg/kg on a dry weight basis. Typically, the rebaudioside B-enriched composition has an undetectable amount of stevia plant-derived contaminants.

Such a steviol glycoside composition can be a rebaudioside C-enriched composition having greater than 15% rebaudioside C and be incorporated into the food product such that the amount of rebaudioside C in the product is from 20-600 mg/kg, e.g., 100-600 mg/kg, 20-100 mg/kg, 20-95 mg/kg, 20-250 mg/kg, 50-75 mg/kg or 50-95 mg/kg on a dry weight basis. Typically, the rebaudioside C-enriched composition has an undetectable amount of stevia plant-derived contaminants.

Such a steviol glycoside composition can be a rebaudioside D-enriched composition having greater than 3% rebaudioside D and be incorporated into the food product such that the amount of rebaudioside D in the product is from 25-1600 mg/kg, e.g., 100-500 mg/kg, 25-100 mg/kg, 250-1000 mg/kg, 50-500 mg/kg or 500-1000 mg/kg on a dry weight basis. Typically, the rebaudioside D-enriched composition has an undetectable amount of stevia plant-derived contaminants.

Such a steviol glycoside composition can be a rebaudioside E-enriched composition having greater than 3% rebaudioside E and be incorporated into the food product such that the amount of rebaudioside E in the product is from 25-1600 mg/kg, e.g., 100-500 mg/kg, 25-100 mg/kg, 250-1000 mg/kg, 50-500 mg/kg or 500-1000 mg/kg on a dry weight basis. Typically, the rebaudioside E-enriched composition has an undetectable amount of stevia plant-derived contaminants.

Such a steviol glycoside composition can be a rebaudioside F-enriched composition having greater than 4% rebaudioside F and be incorporated into the food product such that the amount of rebaudioside F in the product is from 25-1000 mg/kg, e.g., 100-600 mg/kg, 25-100 mg/kg, 25-95 mg/kg, 50-75 mg/kg or 50-95 mg/kg on a dry weight basis. Typically, the rebaudioside F-enriched composition has an undetectable amount of stevia plant-derived contaminants.

Such a steviol glycoside composition can be a dulcoside A-enriched composition having greater than 4% dulcoside A and be incorporated into the food product such that the amount of dulcoside A in the product is from 25-1000 mg/kg, e.g., 100-600 mg/kg, 25-100 mg/kg, 25-95 mg/kg, 50-75 mg/kg or 50-95 mg/kg on a dry weight basis. Typically, the dulcoside A-enriched composition has an undetectable amount of stevia plant-derived contaminants.

Such a steviol glycoside composition can be a composition enriched for rubusoside xylosylated on either of the two positions—the 13-O-glucose or the 19-O-glucose. Such a composition can have greater than 4% of the xylosylated rubusoside compound, and can be incorporated into the food product such that the amount of xylosylated rubusoside compound in the product is from 25-1000 mg/kg, e.g., 100-600 mg/kg, 25-100 mg/kg, 25-95 mg/kg, 50-75 mg/kg or 50-95 mg/kg on a dry weight basis. Typically, the xylosylated rubusoside enriched composition has an undetectable amount of stevia plant-derived contaminants.

Such a steviol glycoside composition can be a composition enriched for compounds rhamnosylated on either of the two positions—the 13-O-glucose or the 19-O-glucose, or compounds containing one rhamnose and multiple glucoses (e.g., steviol 13-O-1,3-diglycoside-1,2-rhamnoside). Such a composition can have greater than 4% of the rhamnosylated compound, and can be incorporated into the food product such that the amount of rhamnosylated compound in the product is from 25-1000 mg/kg, e.g., 100-600 mg/kg, 25-100 mg/kg, 25-95 mg/kg, 50-75 mg/kg or 50-95 mg/kg on a dry weight basis. Typically, the composition enriched for rhamnosylated compounds has as an undetectable amount of stevia plant-derived contaminants.

In some embodiments, a substantially pure steviol or steviol glycoside is incorporated into a tabletop sweetener or “cup-for-cup” product. Such products typically are diluted to the appropriate sweetness level with one or more bulking agents, e.g., maltodextrins, known to those skilled in the art. Steviol glycoside compositions enriched for rebaudioside A, rebaudioside C, rebaudioside D, rebaudioside E, rebaudioside F, dulcoside A, or rhamnosylated or xylosylated compounds, can be package in a sachet, for example, at from 10,000 to 30,000 mg steviol glycoside/kg product on a dry weight basis, for tabletop use.

### EXAMPLES

### Example 1

### Strain Engineering of EFSC 2772

Strain Construction of Saccharomyces cerevisiae EFSC2772

EFSC2772 yeast strain is derived from a wild type Saccharomyces cerevisiae strain containing three auxotrophic modifications, namely the deletions of URA3, LEU2 and HIS3. The strain can be manipulated using standard genetic methods and can be used as a regular diploid or haploid yeast strain. EFSC2772 has been converted to a steviol glycoside producing yeast by genomic-integration of four DNA constructs. Each construct contains multiple genes that were introduced into the yeast genome by homologous recombination. Furthermore, construct one and two were assembled by homologous recombination.

The first construct contains eight genes and is inserted in the DPP1 locus and disrupts and partially deletes DPP1 (phosphatase). The DNA inserted contains: the Ashbya gossypii TEF promoter expressing the natMX gene (selectable marker) followed by the TEF terminator from A. gossypii; Gene Art codon optimized Stevia rebaudiana UGT85C2 (SEQ ID NO:2, codes for GenBank AAR06916.1) expressed from the native yeast GPD1 promoter and followed by the native yeast CYC1 terminator; S. rebaudiana CPR-8 (SEQ ID NOS:3 and 4) expressed using the native yeast TPI1 promoter followed by the native yeast TDH1 terminator; Arabidopsis thaliana Kaurene synthase (GenBank AEE36246.1 coded for by SEQ ID NO:5) expressed from the native yeast PDC1 promoter and followed by the native yeast FBA1 terminator; Synechococcus sp. GGPPS (GenBank ABC98596.1, gene sequence 86553638) expressed using the native yeast TEF2 promoter and followed by the native yeast PGI1 terminator; DNA2.0 codon-optimized S. rebaudiana KAHe1 (SEQ ID NOS:6 and 7), expressed from the native yeast TEF1 promoter and followed by the native yeast ENO2 terminator; S. rebaudiana KO-1 (GenBank ABA42921.1, gi 76446107) expressed using the native yeast FBA1 promoter and followed by the native yeast TDH2 terminator; and Zea mays truncated CDPS (SEQ ID NOS:8 and 9) expressed using the native yeast PGK1 promoter and followed by the native yeast ADH2 terminator.

The second construct was inserted at the YPRCΔ15 locus and contains the TEF1 promoter from A. gossypii in front of the kanMX gene (selectable marker) followed by the TEF1 terminator from A. gossypii, the Gene Art codon optimized A. thaliana ATR2 (SEQ ID NOS:10 and 11) expressed from the native yeast PGK1 promoter followed by the native yeast ADH2 terminator, S. rebaudiana UGT74G1 (GenBank AAR06920.1) expressed from the native yeast TPI1 promoter followed by the native yeast TDH1 terminator, Gene Art codon-optimized S. rebaudiana UGT76G1 (SEQ ID NO:12, codes for GenBank AAR06912) expressed from the native yeast TEF1 promoter followed by the native yeast ENO2 terminator, and GeneArt codon-optimized S. rebaudiana UGT91D2e-b (SEQ ID NO:13 with the following changes: C631A/T857C (nucleotide numbering) and amino acid modifications L211M and V286A) expressed from the native yeast GPD1 promoter and followed by the native yeast CYC1 terminator.

The first and the second construct were combined in the same spore clone by mating and dissection. This yeast strain was subsequently transformed with construct three and four in two successive events.

Construct three was integrated between genes PRP5 and YBR238C and contained the Kluyveromyces lactis LEU2 promoter expressing the K. lactis LEU2 gene followed by the LEU2 terminator from K. lactis, the native yeast GPD1 promoter expressing the DNA2.0-optimized S. rebaudiana KAHe1 followed by the native yeast CYC1 terminator, and the native yeast TPI1 promoter expressing the Zea mays truncated CDPS followed by the native yeast TPI1 terminator. Construct four was integrated in the genome between genes ECM3 and YOR093C with an expression cassette containing the TEF promoter from A. gossypii expressing the K. pneumoniae hphMX gene followed by the TEF1 terminator from A. gossypii, Synechococcus sp. GGPPS expressed from the native yeast GPD1 promoter followed by the native yeast CYC1 terminator, and the native yeast TPI1 promoter expressing the A. thaliana Kaurene synthase followed by the native yeast TPI1 terminator.

The strain was made prototrophic by introduction of the two plasmids p413TEF (public domain CEN/ARS shuttle plasmid with HIS3 marker) and p416-TEF (public domain CEN/ARS shuttle plasmid with URA3 marker) by transformation, and designated EFSC2772.

SEQ ID NO: 2 (Stevia rebaudiana UGT85C2)
ATGGATGCAATGGCAACTACTGAGAAAAAGCCTCATGTGATCTTCATTCCATTTCCTGC
ACAATCTCACATAAAGGCAATGCTAAAGTTAGCACAACTATTACACCATAAGGGATTACA
GATAACTTTCGTGAATACCGACTTCATCCATAATCAATTTCTGGAATCTAGTGGCCCTCA
TTGTTTGGACGGAGCCCCAGGGTTTAGATTCGAAACAATTCCTGACGGTGTTTCACATT
CCCCAGAGGCCTCCATCCCAATAAGAGAGAGTTTACTGAGGTCAATAGAAACCAACTTT
TTGGATCGTTTCATTGACTTGGTCACAAAACTTCCAGACCCACCAACTTGCATAATCTCT
GATGGCTTTCTGTCAGTGTTTACTATCGACGCTGCCAAAAAGTTGGGTATCCCAGTTAT
GATGTACTGGACTCTTGCTGCATGCGGTTTCATGGGTTTCTATCACATCCATTCTCTTAT
CGAAAAGGGTTTTGCTCCACTGAAAGATGCATCATACTTAACCAACGGCTACCTGGATA
CTGTTATTGACTGGGTACCAGGTATGGAAGGTATAAGACTTAAAGATTTTCCTTTGGATT
GGTCTACAGACCTTAATGATAAAGTATTGATGTTTACTACAGAAGCTCCACAAAGATCTC
ATAAGGTTTCACATCATATCTTTCACACCTTTGATGAATTGGAACCATCAATCATCAAAA
CCTTGTCTCTAAGATACAATCATATCTACACTATTGGTCCATTACAATTACTTCTAGATCA
AATTCCTGAAGAGAAAAAGCAAACTGGTATTACATCCTTACACGGCTACTCTTTAGTGAA
AGAGGAACCAGAATGTTTTCAATGGCTACAAAGTAAAGAGCCTAATTCTGTGGTCTACG
TCAACTTCGGAAGTACAACAGTCATGTCCTTGGAAGATATGACTGAATTTGGTTGGGGC
CTTGCTAATTCAAATCATTACTTTCTATGGATTATCAGGTCCAATTTGGTAATAGGGGAA
AACGCCGTATTACCTCCAGAATTGGAGGAACACATCAAAAAGAGAGGTTTCATTGCTTC
CTGGTGTTCTCAGGAAAAGGTATTGAAACATCCTTCTGTTGGTGGTTTCCTTACTCATTG
CGGTTGGGGCTCTACAATCGAATCACTAAGTGCAGGAGTTCCAATGATTTGTTGGCCAT
ATTCATGGGACCAACTTACAAATTGTAGGTATATCTGTAAAGAGTGGGAAGTTGGATTA
GAAATGGGAACAAAGGTTAAACGTGATGAAGTGAAAAGATTGGTTCAGGAGTTGATGG
GGGAAGGTGGCCACAAGATGAGAAACAAGGCCAAAGATTGGAAGGAAAAAGCCAGAAT
TGCTATTGCTCCTAACGGGTCATCCTCTCTAAACATTGATAAGATGGTCAAAGAGATTAC
AGTCTTAGCCAGAAACTAA
SEQ ID NO: 3 (Stevia rebaudiana CPR8)
MQSNSVKISPLDLVTALFSGKVLDTSNASESGESAMLPTIAMIMENRELLMILTTSVAVLIGC
VVVLVWRRSSTKKSALEPPVIVVPKRVQEEEVDDGKKKVTVFFGTQTGTAEGFAKALVEEA
KARYEKAVFKVIDLDDYAADDDEYEEKLKKESLAFFFLATYGDGEPTDNAARFYKWFTEGD
AKGEWLNKLQYGVFGLGNRQYEHFNKIAKVVDDGLVEQGAKRLVPVGLGDDDQCIEDDFT
AWKELVWPELDQLLRDEDDTTVATPYTAAVAEYRVVFHEKPDALSEDYSYTNGHAVHDAQ
HPCRSNVAVKKELHSPESDRSCTHLEFDISNTGLSYETGDHVGVYCENLSEVVNDAERLVG
LPPDTYSSIHTDSEDGSPLGGASLPPPFPPCTLRKALTCYADVLSSPKKSALLALAAHATDP
SEADRLKFLASPAGKDEYSQWIVASQRSLLEVMEAFPSAKPSLGVFFASVAPRLQPRYYSIS
SSPKMAPDRIHVTCALVYEKTPAGRIHKGVCSTWMKNAVPMTESQDCSWAPIYVRTSNFR
LPSDPKVPVIMIGPGTGLAPFRGFLQERLALKEAGTDLGLSILFFGCRNRKVDFIYENELNNF
VETGALSELIVAFSREGPTKEYVQHKMSEKASDIWNLLSEGAYLYVC
SEQ ID NO: 4 (Stevia rebaudiana CPR8; CPR
cloned from Stevia rebaudiana cDNA)
ATGCAATCTAACTCCGTGAAGATTTCGCCGCTTGATCTGGTAACTGCGCTGTTTAGCGG
CAAGGTTTTGGACACATCGAACGCATCGGAATCGGGAGAATCTGCTATGCTGCCGACT
ATAGCGATGATTATGGAGAATCGTGAGCTGTTGATGATACTCACAACGTCGGTTGCTGT
ATTGATCGGATGCGTTGTCGTTTTGGTGTGGCGGAGATCGTCTACGAAGAAGTCGGCG
TTGGAGCCACCGGTGATTGTGGTTCCGAAGAGAGTGCAAGAGGAGGAAGTTGATGATG
GTAAGAAGAAAGTTACGGTTTTCTTCGGCACCCAAACTGGAACAGCTGAAGGCTTCGCT
AAGGCACTTGTTGAGGAAGCTAAAGCTCGATATGAAAAGGCTGTCTTTAAAGTAATTGA
TTTGGATGATTATGCTGCTGATGACGATGAGTATGAGGAGAAACTAAAGAAAGAATCTT
TGGCCTTTTTCTTTTTGGCTACGTATGGAGATGGTGAGCCAACAGATAATGCTGCCAGA
TTTTATAAATGGTTTACTGAGGGAGATGCGAAAGGAGAATGGCTTAATAAGCTTCAATAT
GGAGTATTTGGTTTGGGTAACAGACAATATGAACATTTTAACAAGATCGCAAAAGTGGTT
GATGATGGTCTTGTAGAACAGGGTGCAAAGCGTCTTGTTCCTGTTGGACTTGGAGATGA
TGATCAATGTATTGAAGATGACTTCACCGCATGGAAAGAGTTAGTATGGCCGGAGTTGG
ATCAATTACTTCGTGATGAGGATGACACAACTGTTGCTACTCCATACACAGCTGCTGTT
GCAGAATATCGCGTTGTTTTTCATGAAAAACCAGACGCGCTTTCTGAAGATTATAGTTAT
ACAAATGGCCATGCTGTTCATGATGCTCAACATCCATGCAGATCCAACGTGGCTGTCAA
AAAGGAACTTCATAGTCCTGAATCTGACCGGTCTTGCACTCATCTTGAATTTGACATCTC
GAACACCGGACTATCATATGAAACTGGGGACCATGTTGGAGTTTACTGTGAAAACTTGA
GTGAAGTTGTGAATGATGCTGAAAGATTAGTAGGATTACCACCAGACACTTACTCCTCC
ATCCACACTGATAGTGAAGACGGGTCGCCACTTGGCGGAGCCTCATTGCCGCCTCCTT
TCCCGCCATGCACTTTAAGGAAAGCATTGACGTGTTATGCTGATGTTTTGAGTTCTCCC
AAGAAGTCGGCTTTGCTTGCACTAGCTGCTCATGCCACCGATCCCAGTGAAGCTGATA
GATTGAAATTTCTTGCATCCCCCGCCGGAAAGGATGAATATTCTCAATGGATAGTTGCA
AGCCAAAGAAGTCTCCTTGAAGTCATGGAAGCATTCCCGTCAGCTAAGCCTTCACTTGG
TGTTTTCTTTGCATCTGTTGCCCCGCGCTTACAACCAAGATACTACTCTATTTCTTCCTC
ACCCAAGATGGCACCGGATAGGATTCATGTTACATGTGCATTAGTCTATGAGAAAACAC
CTGCAGGCCGCATCCACAAAGGAGTTTGTTCAACTTGGATGAAGAACGCAGTGCCTAT
GACCGAGAGTCAAGATTGCAGTTGGGCCCCAATATACGTCCGAACATCCAATTTCAGAC
TACCATCTGACCCTAAGGTCCCGGTTATCATGATTGGACCTGGCACTGGTTTGGCTCCT
TTTAGAGGTTTCCTTCAAGAGCGGTTAGCTTTAAAGGAAGCCGGAACTGACCTCGGTTT
ATCCATTTTATTCTTCGGATGTAGGAATCGCAAAGTGGATTTCATATATGAAAACGAGCT
TAACAACTTTGTGGAGACTGGTGCTCTTTCTGAGCTTATTGTTGCTTTCTCCCGTGAAG
GCCCGACTAAGGAATATGTGCAACACAAGATGAGTGAGAAGGCTTCGGATATCTGGAA
CTTGCTTTCTGAAGGAGCATATTTATACGTATGTGGTGATGCCAAAGGCATGGCCAAAG
ATGTACATCGAACCCTCCACACAATTGTGCAAGAACAGGGATCTCTTGACTCGTCAAAG
GCAGAACTCTACGTGAAGAATCTACAAATGTCAGGAAGATACCTCCGTGACGTTTGGTA
A
SEQ ID NO: 5 (Arabidopsis thaliana Kaurene synthase)
CGTCAGTCATCAAGGCTAATTCGTCGCGAGTTGCTACGACGCCGTTTCGGTTGCTTCTG
GTTTCTTTATGTCTATCAACCTTCGCTCCTCCGGTTGTTCGTCTCCGATCTCAGCTACTT
TGGAACGAGGATTGGACTCAGAAGTACAGACAAGAGCTAACAATGTGAGCTTTGAGCA
AACAAAGGAGAAGATTAGGAAGATGTTGGAGAAAGTGGAGCTTTCTGTTTCGGCCTAC
GATACTAGTTGGGTAGCAATGGTTCCATCACCGAGCTCCCAAAATGCTCCACTTTTCCC
ACAGTGTGTGAAATGGTTATTGGATAATCAACATGAAGATGGATCTTGGGGACTTGATA
ACCATGACCATCAATCTCTTAAGAAGGATGTGTTATCATCTACACTGGCTAGTATCCTCG
CGTTAAAGAAGTGGGGAATTGGTGAAAGACAAATAAACAAGGGTCTCCAGTTTATTGAG
CTGAATTCTGCATTAGTCACTGATGAAACCATACAGAAACCAACAGGGTTTGATATTATA
TTTCCTGGGATGATTAAATATGCTAGAGATTTGAATCTGACGATTCCATTGGGCTCAGAA
GTGGTGGATGACATGATACGAAAAAGAGATCTGGATCTTAAATGTGATAGTGAAAAGTT
TTCAAAGGGAAGAGAAGCATATCTGGCCTATGTTTTAGAGGGGACAAGAAACCTAAAAG
ATTGGGATTTGATAGTCAAATATCAAAGGAAAAATGGGTCACTGTTTGATTCTCCAGCCA
CAACAGCAGCTGCTTTTACTCAGTTTGGGAATGATGGTTGTCTCCGTTATCTCTGTTCTC
TCCTTCAGAAATTCGAGGCTGCAGTTCCTTCAGTTTATCCATTTGATCAATATGCACGCC
TTAGTATAATTGTCACTCTTGAAAGCTTAGGAATTGATAGAGATTTCAAAACCGAAATCA
AAAGCATATTGGATGAAACCTATAGATATTGGCTTCGTGGGGATGAAGAAATATGTTTG
GACTTGGCCACTTGTGCTTTGGCTTTCCGATTATTGCTTGCTCATGGCTATGATGTGTCT
TACGATCCGCTAAAACCATTTGCAGAAGAATCTGGTTTCTCTGATACTTTGGAAGGATAT
GTTAAGAATACGTTTTCTGTGTTAGAATTATTTAAGGCTGCTCAAAGTTATCCACATGAA
TCAGCTTTGAAGAAGCAGTGTTGTTGGACTAAACAATATCTGGAGATGGAATTGTCCAG
CTGGGTTAAGACCTCTGTTCGAGATAAATACCTCAAGAAAGAGGTCGAGGATGCTCTTG
CTTTTCCCTCCTATGCAAGCCTAGAAAGATCAGATCACAGGAGAAAAATACTCAATGGT
TCTGCTGTGGAAAACACCAGAGTTACAAAAACCTCATATCGTTTGCACAATATTTGCACC
TCTGATATCCTGAAGTTAGCTGTGGATGACTTCAATTTCTGCCAGTCCATACACCGTGA
AGAAATGGAACGTCTTGATAGGTGGATTGTGGAGAATAGATTGCAGGAACTGAAATTTG
CCAGACAGAAGCTGGCTTACTGTTATTTCTCTGGGGCTGCAACTTTATTTTCTCCAGAA
CTATCTGATGCTCGTATATCGTGGGCCAAAGGTGGAGTACTTACAACGGTTGTAGACGA
CTTCTTTGATGTTGGAGGGTCCAAAGAAGAACTGGAAAACCTCATACACTTGGTCGAAA
AGTGGGATTTGAACGGTGTTCCTGAGTACAGCTCAGAACATGTTGAGATCATATTCTCA
GTTCTAAGGGACACCATTCTCGAAACAGGAGACAAAGCATTCACCTATCAAGGACGCAA
TGTGACACACCACATTGTGAAAATTTGGTTGGATCTGCTCAAGTCTATGTTGAGAGAAG
CCGAGTGGTCCAGTGACAAGTCAACACCAAGCTTGGAGGATTACATGGAAAATGCGTA
CATATCATTTGCATTAGGACCAATTGTCCTCCCAGCTACCTATCTGATCGGACCTCCACT
TCCAGAGAAGACAGTCGATAGCCACCAATATAATCAGCTCTACAAGCTCGTGAGCACTA
TGGGTCGTCTTCTAAATGACATACAAGGTTTTAAGAGAGAAAGCGCGGAAGGGAAGCT
GAATGCGGTTTCATTGCACATGAAACACGAGAGAGACAATCGCAGCAAAGAAGTGATC
ATAGAATCGATGAAAGGTTTAGCAGAGAGAAAGAGGGAAGAATTGCATAAGCTAGTTTT
GGAGGAGAAAGGAAGTGTGGTTCCAAGGGAATGCAAAGAAGCGTTCTTGAAAATGAGC
AAAGTGTTGAACTTATTTTACAGGAAGGACGATGGATTCACATCAAATGATCTGATGAGT
CTTGTTAAATCAGTGATCTACGAGCCTGTTAGCTTACAGAAAGAATCTTTAACTTGATCC
AAGTTGATCTGGCAGGTAAACTCAGTAAATGAAAATAAGACTTTGGTCTTCTTCTTTGTT
GCTTCAGAACAAGAAGAG
SEQ ID NO: 6 (Stevia rebaudiana KAHe1)
ATGGAAGCCTCTTACCTATACATTTCTATTTTGCTTTTACTGGCATCATACCTGTTCACCA
CTCAACTTAGAAGGAAGAGCGCTAATCTACCACCAACCGTGTTTCCATCAATACCAATC
ATTGGACACTTATACTTACTCAAAAAGCCTCTTTATAGAACTTTAGCAAAAATTGCCGCT
AAGTACGGACCAATACTGCAATTACAACTCGGCTACAGACGTGTTCTGGTGATTTCCTC
ACCATCAGCAGCAGAAGAGTGCTTTACCAATAACGATGTAATCTTCGCAAATAGACCTA
AGACATTGTTTGGCAAAATAGTGGGTGGAACATCCCTTGGCAGTTTATCCTACGGCGAT
CAATGGCGTAATCTAAGGAGAGTAGCTTCTATCGAAATCCTATCAGTTCATAGGTTGAA
CGAATTTCATGATATCAGAGTGGATGAGAACAGATTGTTAATTAGAAAACTTAGAAGTTC
ATCTTCTCCTGTTACTCTTATAACAGTCTTTTATGCTCTAACATTGAACGTCATTATGAGA
ATGATCTCTGGCAAAAGATATTTCGACAGTGGGGATAGAGAATTGGAGGAGGAAGGTA
AGAGATTTCGAGAAATCTTAGACGAAACGTTGCTTCTAGCCGGTGCTTCTAATGTTGGC
GACTACTTACCAATATTGAACTGGTTGGGAGTTAAGTCTCTTGAAAAGAAATTGATCGCT
TTGCAGAAAAAGAGAGATGACTTTTTCCAGGGTTTGATTGAACAGGTTAGAAAATCTCG
TGGTGCTAAAGTAGGCAAAGGTAGAAAAACGATGATCGAACTCTTATTATCTTTGCAAG
AGTCAGAACCTGAGTACTATACAGATGCTATGATAAGATCTTTTGTCCTAGGTCTGCTG
GCTGCAGGTAGTGATACTTCAGCGGGCACTATGGAATGGGCCATGAGCTTACTGGTCA
ATCACCCACATGTATTGAAGAAAGCTCAAGCTGAAATCGATAGAGTTATCGGTAATAAC
AGATTGATTGACGAGTCAGACATTGGAAATATCCCTTACATCGGGTGTATTATCAATGAA
ACTCTAAGACTCTATCCAGCAGGGCCATTGTTGTTCCCACATGAAAGTTCTGCCGACTG
CGTTATTTCCGGTTACAATATACCTAGAGGTACAATGTTAATCGTAAACCAATGGGCGAT
TCATCACGATCCTAAAGTCTGGGATGATCCTGAAACCTTTAAACCTGAAAGATTTCAAG
GATTAGAAGGAACTAGAGATGGTTTCAAACTTATGCCATTCGGTTCTGGGAGAAGAGGA
TGTCCAGGTGAAGGTTTGGCAATAAGGCTGTTAGGGATGACACTAGGCTCAGTGATCC
AATGTTTTGATTGGGAGAGAGTAGGAGATGAGATGGTTGACATGACAGAAGGTTTGGG
TGTCACACTTCCTAAGGCCGTTCCATTAGTTGCCAAATGTAAGCCACGTTCCGAAATGA
CTAATCTCCTATCCGAACTTTAA
SEQ ID NO: 7 (Stevia KAH (SrKAHe1) amino acid sequence)
MEASYLYISILLLLASYLFTTQLRRKSANLPPTVFPSIPIIGHLYLLKKPLYRTLAKIAAKYGPILQ
LQLGYRRVLVISSPSAAEECFTNNDVIFANRPKTLFGKIVGGTSLGSLSYGDQWRNLRRVAS
IEILSVHRLNEFHDIRVDENRLLIRKLRSSSSPVTLITVFYALTLNVIMRMISGKRYFDSGDREL
EEEGKRFREILDETLLLAGASNVGDYLPILNWLGVKSLEKKLIALQKKRDDFFQGLIEQVRKS
RGAKVGKGRKTMIELLLSLQESEPEYYTDAMIRSFVLGLLAAGSDTSAGTMEWAMSLLVNH
PHVLKKAQAEIDRVIGNNRLIDESDIGNIPYIGCIINETLRLYPAGPLLFPHESSADCVISGYNIP
RGTMLIVNQWAIHHDPKVWDDPETFKPERFQGLEGTRDGFKLMPFGSGRRGCPGEGLAIR
LLGMTLGSVIQCFDWERVGDEMVDMTEGLGVTLPKAVPLVAKCKPRSEMTNLLSEL
SEQ ID NO: 8 (DNA sequence of truncated Zea mays CDPA;
underlined portions removed from native sequence)
ATGGTTTTGTCTTCTTCTTGTACTACAGTACCACACTTATCTTCATTAGCTGTCGTGCAA
CTTGGTCCTTGGAGCAGTAGGATTAAAAAGAAAACCGATACTGTTGCAGTACCAGCCGC
TGCAGGAAGGTGGAGAAGGGCCTTGGCTAGAGCACAGCACACATCAGAATCCGCAGC
TGTCGCAAAGGGCAGCAGTTTGACCCCTATAGTGAGAACTGACGCTGAGTCAAGGAGA
ACAAGATGGCCAACCGATGACGATGACGCCGAACCTTTAGTGGATGAGATCAGGGCAA
TGCTTACTTCCATGTCTGATGGTGACATTTCCGTGAGCGCATACGATACAGCCTGGGTC
GGATTGGTTCCAAGATTAGACGGCGGTGAAGGTCCTCAATTTCCAGCAGCTGTGAGAT
GGATAAGAAATAACCAGTTGCCTGACGGAAGTTGGGGCGATGCCGCATTATTCTCTGC
CTATGACAGGCTTATCAATACCCTTGCCTGCGTTGTAACTTTGACAAGGTGGTCCCTAG
AACCAGAGATGAGAGGTAGAGGACTATCTTTTTTGGGTAGGAACATGTGGAAATTAGCA
ACTGAAGATGAAGAGTCAATGCCTATTGGCTTCGAATTAGCATTTCCATCTTTGATAGAG
CTTGCTAAGAGCCTAGGTGTCCATGACTTCCCTTATGATCACCAGGCCCTACAAGGAAT
CTACTCTTCAAGAGAGATCAAAATGAAGAGGATTCCAAAAGAAGTGATGCATACCGTTC
CAACATCAATATTGCACAGTTTGGAGGGTATGCCTGGCCTAGATTGGGCTAAACTACTT
AAACTACAGAGCAGCGACGGAAGTTTTTTGTTCTCACCAGCTGCCACTGCATATGCTTT
AATGAATACCGGAGATGACAGGTGTTTTAGCTACATCGATAGAACAGTAAAGAAATTCA
ACGGCGGCGTCCCTAATGTTTATCCAGTGGATCTATTTGAACATATTTGGGCCGTTGAT
AGACTTGAAAGATTAGGAATCTCCAGGTACTTCCAAAAGGAGATCGAACAATGCATGGA
TTATGTAAACAGGCATTGGACTGAGGACGGTATTTGTTGGGCAAGGAACTCTGATGTCA
AAGAGGTGGACGACACAGCTATGGCCTTTAGACTTCTTAGGTTGCACGGCTACAGCGT
CAGTCCTGATGTGTTTAAAAACTTCGAAAAGACGGTGAATTTTTCGCATTTGTCGGACA
GTCTAATCAAGCTGTTACCGGTATGTACAACTTAAACAGAGCAAGCCAGATATCCTTCC
CAGGCGAGGATGTGCTTCATAGAGCTGGTGCCTTCTCATATGAGTTCTTGAGGAGAAAA
GAAGCAGAGGGAGCTTTGAGGGACAAGTGGATCATTTCTAAAGATCTACCTGGTGAAG
TTGTGTATACTTTGGATTTTCCATGGTACGGCAACTTACCTAGAGTCGAGGCCAGAGAC
TACCTAGAGCAATACGGAGGTGGTGATGACGTTTGGATTGGCAAGACATTGTATAGGAT
GCCACTTGTAAACAATGATGTATATTTGGAATTGGCAAGAATGGATTTCAACCACTGCCA
GGCTTTGCATCAGTTAGAGTGGCAAGGACTAAAAAGATGGTATACTGAAAATAGGTTGA
TGGACTTTGGTGTCGCCCAAGAAGATGCCCTTAGAGCTTATTTTCTTGCAGCCGCATCT
GTTTACGAGCCTTGTAGAGCTGCCGAGAGGCTTGCATGGGCTAGAGCCGCAATACTAG
CTAACGCCGTGAGCACCCACTTAAGAAATAGCCCATCATTCAGAGAAAGGTTAGAGCAT
TCTCTTAGGTGTAGACCTAGTGAAGAGACAGATGGCTCCTGGTTTAACTCCTCAAGTGG
CTCTGATGCAGTTTTAGTAAAGGCTGTCTTAAGACTTACTGATTCATTAGCCAGGGAAG
CACAGCCAATCCATGGAGGTGACCCAGAAGATATTATACACAAGTTGTTAAGATCTGCT
TGGGCCGAGTGGGTTAGGGAAAAGGCAGACGCTGCCGATAGCGTGTGCAATGGTAGT
TCTGCAGTAGAACAAGAGGGATCAAGAATGGTCCATGATAAACAGACCTGTCTATTATT
GGCTAGAATGATCGAAATTTCTGCCGGTAGGGCAGCTGGTGAAGCAGCCAGTGAGGAC
GGCGATAGAAGAATAATTCAATTAACAGGCTCCATCTGCGACAGTCTTAAGCAAAAAAT
GCTAGTTTCACAGGACCCTGAAAAAAATGAAGAGATGATGTCTCACGTGGATGACGAAT
TGAAGTTGAGGATTAGAGAGTTCGTTCAATATTTGCTTAGACTAGGTGAAAAAAAGACT
GGATCTAGCGAAACCAGGCAAACATTTTTAAGTATAGTGAAATCATGTTACTATGCTGCT
CATTGCCCACCTCATGTCGTTGATAGACACATTAGTAGAGTGATTTTCGAGCCAGTAAG
TGCCGCAAAGTAACCGCGG
SEQ ID NO: 9 (Protein sequence of truncated Zea mays
CDPS; underlined portions absent from truncated version)
MVLSSSCTTVPHLSSLAVVQLGPWSSRIKKKTDTVAVPAAAGRWRRALARAQHTSESAAV
AKGSSLTPIVRTDAESRRTRWPTDDDDAEPLVDEIRAMLTSMSDGDISVSAYDTAWVGLVP
RLDGGEGPQFPAAVRWIRNNQLPDGSWGDAALFSAYDRLINTLACVVTLTRWSLEPEMRG
RGLSFLGRNMWKLATEDEESMPIGFELAFPSLIELAKSLGVHDFPYDHQALQGIYSSREIKM
KRIPKEVMHTVPTSILHSLEGMPGLDWAKLLKLQSSDGSFLFSPAATAYALMNTGDDRCFS
YIDRTVKKFNGGVPNVYPVDLFEHIWAVDRLERLGISRYFQKEIEQCMDYVNRHWTEDGIC
WARNSDVKEVDDTAMAFRLLRLHGYSVSPDVFKNFEKDGEFFAFVGQSNQAVTGMYNLN
RASQISFPGEDVLHRAGAFSYEFLRRKEAEGALRDKWIISKDLPGEVVYTLDFPWYGNLPR
VEARDYLEQYGGGDDVWIGKTLYRMPLVNNDVYLELARMDFNHCQALHQLEWQGLKRWY
TENRLMDFGVAQEDALRAYFLAAASVYEPCRAAERLAWARAAILANAVSTHLRNSPSFRER
LEHSLRCRPSEETDGSWFNSSSGSDAVLVKAVLRLTDSLAREAQPIHGGDPEDIIHKLLRSA
WAEWVREKADAADSVCNGSSAVEQEGSRMVHDKQTCLLLARMIEISAGRAAGEAASEDG
DRRIIQLTGSICDSLKQKMLVSQDPEKNEEMMSHVDDELKLRIREFVQYLLRLGEKKTGSSE
TRQTFLSIVKSCYYAAHCPPHVVDRHISRVIFEPVSAAK
SEQ ID NO: 10 (A. thaliana CPR polypeptide encoded by ATR2)
MSSSSSSSTSMIDLMAAIIKGEPVIVSDPANASAYESVAAELSSMLIENRQFAMIVTTSIAVLIG
CIVMLVWRRSGSGNSKRVEPLKPLVIKPREEEIDDGRKKVTIFFGTQTGTAEGFAKALGEEA
KARYEKTRFKIVDLDDYAADDDEYEEKLKKEDVAFFFLATYGDGEPTDNAARFYKWFTEGN
DRGEWLKNLKYGVFGLGNRQYEHFNKVAKVVDDILVEQGAQRLVQVGLGDDDQCIEDDFT
AWREALWPELDTILREEGDTAVATPYTAAVLEYRVSIHDSEDAKFNDITLANGNGYTVFDAQ
HPYKANVAVKRELHTPESDRSCIHLEFDIAGSGLTMKLGDHVGVLCDNLSETVDEALRLLD
MSPDTYFSLHAEKEDGTPISSSLPPPFPPCNLRTALTRYACLLSSPKKSALVALAAHASDPT
EAERLKHLASPAGKDEYSKWVVESQRSLLEVMAEFPSAKPPLGVFFAGVAPRLQPRFYSIS
SSPKIAETRIHVTCALVYEKMPTGRIHKGVCSTWMKNAVPYEKSEKLFLGRPIFVRQSNFKL
PSDSKVPIIMIGPGTGLAPFRGFLQERLALVESGVELGPSVLFFGCRNRRMDFIYEEELQRF
VESGALAELSVAFSREGPTKEYVQHKMMDKASDIWNMISQGAYLYVCGDAKGMARDVHR
SLHTIAQEQGSMDSTKAEGFVKNLQTSGRYLRDVW
SEQ ID NO: 11 (ATR2 codon optimized by GeneArt)
ATGTCTTCCTCTTCCTCTTCCAGTACCTCTATGATTGATTTGATGGCTGCTATTATTAAAG
GTGAACCAGTTATCGTCTCCGACCCAGCAAATGCCTCTGCTTATGAATCAGTTGCTGCA
GAATTGTCTTCAATGTTGATCGAAAACAGACAATTCGCCATGATCGTAACTACATCAATC
GCTGTTTTGATCGGTTGTATTGTCATGTTGGTATGGAGAAGATCCGGTAGTGGTAATTC
TAAAAGAGTCGAACCTTTGAAACCATTAGTAATTAAGCCAAGAGAAGAAGAAATAGATG
ACGGTAGAAAGAAAGTTACAATATTTTTCGGTACCCAAACTGGTACAGCTGAAGGTTTT
GCAAAAGCCTTAGGTGAAGAAGCTAAGGCAAGATACGAAAAGACTAGATTCAAGATAGT
CGATTTGGATGACTATGCCGCTGATGACGATGAATACGAAGAAAAGTTGAAGAAAGAAG
ATGTTGCATTTTTCTTTTTGGCAACCTATGGTGACGGTGAACCAACTGACAATGCAGCC
AGATTCTACAAATGGTTTACAGAGGGTAATGATCGTGGTGAATGGTTGAAAAACTTAAA
GTACGGTGTTTTCGGTTTGGGTAACAGACAATACGAACATTTCAACAAAGTTGCAAAGG
TTGTCGACGATATTTTGGTCGAACAAGGTGCTCAAAGATTAGTCCAAGTAGGTTTGGGT
GACGATGACCAATGTATAGAAGATGACTTTACTGCCTGGAGAGAAGCTTTGTGGCCTGA
ATTAGACACAATCTTGAGAGAAGAAGGTGACACCGCCGTTGCTACCCCATATACTGCTG
CAGTATTAGAATACAGAGTTTCCATCCATGATAGTGAAGACGCAAAGTTTAATGATATCA
CTTTGGCCAATGGTAACGGTTATACAGTTTTCGATGCACAACACCCTTACAAAGCTAAC
GTTGCAGTCAAGAGAGAATTACATACACCAGAATCCGACAGAAGTTGTATACACTTGGA
ATTTGATATCGCTGGTTCCGGTTTAACCATGAAGTTGGGTGACCATGTAGGTGTTTTAT
GCGACAATTTGTCTGAAACTGTTGATGAAGCATTGAGATTGTTGGATATGTCCCCTGAC
ACTTATTTTAGTTTGCACGCTGAAAAAGAAGATGGTACACCAATTTCCAGTTCTTTACCA
CCTCCATTCCCTCCATGTAACTTAAGAACAGCCTTGACCAGATACGCTTGCTTGTTATCA
TCCCCTAAAAAGTCCGCCTTGGTTGCTTTAGCCGCTCATGCTAGTGATCCTACTGAAGC
AGAAAGATTGAAACACTTAGCATCTCCAGCCGGTAAAGATGAATATTCAAAGTGGGTAG
TTGAATCTCAAAGATCATTGTTAGAAGTTATGGCAGAATTTCCATCTGCCAAGCCTCCAT
TAGGTGTCTTCTTTGCTGGTGTAGCACCTAGATTGCAACCAAGATTCTACTCAATCAGTT
CTTCACCTAAGATCGCTGAAACTAGAATTCATGTTACATGTGCATTAGTCTACGAAAAGA
TGCCAACCGGTAGAATTCACAAGGGTGTATGCTCTACTTGGATGAAAAATGCTGTTCCT
TACGAAAAATCAGAAAAGTTGTTCTTAGGTAGACCAATCTTCGTAAGACAATCAAACTTC
AAGTTGCCTTCTGATTCAAAGGTTCCAATAATCATGATAGGTCCTGGTACAGGTTTAGC
CCCATTCAGAGGTTTCTTGCAAGAAAGATTGGCTTTAGTTGAATCTGGTGTCGAATTAG
GTCCTTCAGTTTTGTTCTTTGGTTGTAGAAACAGAAGAATGGATTTCATCTATGAAGAAG
AATTGCAAAGATTCGTCGAATCTGGTGCATTGGCCGAATTATCTGTAGCTTTTTCAAGAG
AAGGTCCAACTAAGGAATACGTTCAACATAAGATGATGGATAAGGCATCCGACATATGG
AACATGATCAGTCAAGGTGCTTATTTGTACGTTTGCGGTGACGCAAAGGGTATGGCCAG
AGATGTCCATAGATCTTTGCACACAATTGCTCAAGAACAAGGTTCCATGGATAGTACCA
AAGCTGAAGGTTTCGTAAAGAACTTACAAACTTCCGGTAGATACTTGAGAGATGTCTGG
TGA
SEQ ID NO: 12 (Stevia rebaudiana)
ATGGAAAACAAGACCGAAACAACAGTTAGACGTAGGCGTAGAATCATTCTGTTTCCAGT
ACCTTTTCAAGGGCACATCAATCCAATACTACAACTAGCCAACGTTTTGTACTCTAAAGG
TTTTTCTATTACAATCTTTCACACCAATTTCAACAAACCAAAAACATCCAATTACCCACAT
TTCACATTCAGATTCATACTTGATAATGATCCACAAGATGAACGTATTTCAAACTTACCTA
CCCACGGTCCTTTAGCTGGAATGAGAATTCCAATCATCAATGAACATGGTGCCGATGAG
CTTAGAAGAGAATTAGAGTTACTTATGTTGGCATCCGAAGAGGACGAGGAAGTCTCTTG
TCTGATTACTGACGCTCTATGGTACTTTGCCCAATCTGTGGCTGATAGTTTGAATTTGAG
GAGATTGGTACTAATGACATCCAGTCTGTTTAACTTTCACGCTCATGTTAGTTTACCACA
ATTTGACGAATTGGGATACTTGGACCCTGATGACAAGACTAGGTTAGAGGAACAGGCCT
CTGGTTTTCCTATGTTGAAAGTCAAAGATATCAAGTCTGCCTATTCTAATTGGCAAATCT
TGAAAGAGATCTTAGGAAAGATGATCAAACAGACAAAGGCTTCATCTGGAGTGATTTGG
AACAGTTTCAAAGAGTTAGAAGAGTCTGAATTGGAGACTGTAATCAGAGAAATTCCAGC
ACCTTCATTCCTGATACCATTACCAAAACATTTGACTGCTTCCTCTTCCTCTTTGTTGGAT
CATGACAGAACAGTTTTTCAATGGTTGGACCAACAACCACCTAGTTCTGTTTTGTACGTG
TCATTTGGTAGTACTTCTGAAGTCGATGAAAAGGACTTCCTTGAAATCGCAAGAGGCTT
AGTCGATAGTAAGCAGTCATTCCTTTGGGTCGTGCGTCCAGGTTTCGTGAAAGGCTCAA
CATGGGTCGAACCACTTCCAGATGGTTTTCTAGGCGAAAGAGGTAGAATAGTCAAATGG
GTTCCTCAACAGGAAGTTTTAGCTCATGGCGCTATTGGGGCATTCTGGACTCATTCCGG
ATGGAATTCAACTTTAGAATCAGTATGCGAAGGGGTACCTATGATCTTTTCAGATTTTGG
TCTTGATCAACCACTGAACGCAAGATACATGTCTGATGTTTTGAAAGTGGGTGTATATCT
AGAAAATGGCTGGGAAAGGGGTGAAATAGCTAATGCAATAAGACGTGTTATGGTTGATG
AAGAGGGGGAGTATATCAGACAAAACGCAAGAGTGCTGAAGCAAAAGGCCGACGTTTC
TCTAATGAAGGGAGGCTCTTCATACGAATCCTTAGAATCTCTTGTTTCCTACATTTCATC
ACTGTAA
SEQ ID NO: 13 (Stevia rebaudiana)
MATSDSIVDDRKQLHVATFPWLAFGHILPYLQLSKLIAEKGHKVSFLSTTRNIQRLSSHISPLI
NVVQLTLPRVQELPEDAEATTDVHPEDIPYLKKASDGLQPEVTRFLEQHSPDWIIYDYTHYW
LPSIAASLGISRAHFSVTTPWAIAYMGPSADAMINGSDGRTTVEDLTTPPKWFPFPTKVCWR
KHDLARLVPYKAPGISDGYRMGLVLKGSDCLLSKCYHEFGTQWLPLLETLHQVPVVPVGLL
PPEIPGDEKDETWVSIKKWLDGKQKGSVVYVALGSEVLVSQTEVVELALGLELSGLPFVWA
YRKPKGPAKSDSVELPDGFVERTRDRGLVWTSWAPQLRILSHESVCGFLTHCGSGSIVEG
LMFGHPLIMLPIFGDQPLNARLLEDKQVGIEIPRNEEDGCLTKESVARSLRSVVVEKEGEIYK
ANARELSKIYNDTKVEKEYVSQFVDYLEKNARAVAIDHES

### Example 2

### Fermentation Production of Gram-Scale Samples of Rebaudioside A

The cultivations of strain EFSC2772 were performed in 4 BRAUN fermenters with a total volume of 13 liters and a maximal working volume of 10 liters. The fermenters were controlled by 4 X-Controller units (INFORS). Monitored parameters included temperature, pH, dissolved oxygen (DO), stirring speed, and antifoam addition.

Shake flask cultures (one-stage) were grown in Synthetic Complete Drop-Out Media (SC)+10 g/L succinate and used to seed the fermenters. Approximately 10% v/v from the seed cultures was inoculated into a fermenter and grown in the appropriate Synthetic Complete Drop-Out Media (SC). The pH setpoint was 5 and the temperature setpoint was 30° C., 1 vvm aeration or higher was utilized with high rpms to maintain aerobic conditions. The cultivations were started with a 3.75 liter working volume in a batch mode for about 21 hours. Then the feed of about 5 liters (total) was carried out over approximately 100 hours. The feed contained glucose as the sole carbon and energy source combined with trace metals, vitamins, salts, and amino acids. The feeding rate changed daily to target steadily decreasing growth rates, minimizing ethanol formation.

Immediately following the fermentations, the entire culture broth was harvested and centrifuged for 20 to 25 minutes at 12,500×g. The supernatant was filtered through a 0.45 μm filter, pooled for the next working step and refrigerated at 4° C. Approximately 100 g/L cell dry weight (CDW) biomass titer was reached in successful fermentations which maintained aerobic conditions and were not subjected to extreme pH drops.

### Example 3

### HP-20 Column Enrichment of Rebaudioside A (RebA)

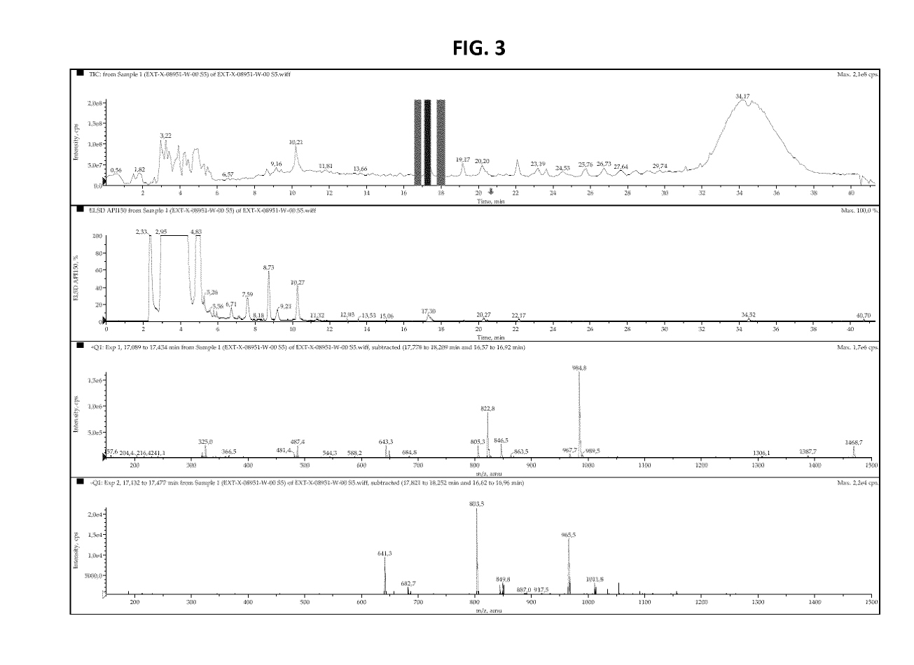

The LC-MS analysis of the fermentation broth showed that the largest contaminants were polar compounds (FIG. 3), and therefore de-fatting with hexane and removal of polyphenols were not necessary in the first isolation step. Reb-A eluted at 17.30 min together with a closely co-eluting by-product visible in the HPLC analysis.

As a result of the analysis it was determined that the first step should be the adsorption of the supernatant on the resin HP-20L (DIAION®, Mitsubishi Chem.). This resin is a synthetic polyaromatic gel used often for adsorption of large molecules in natural products separations due to the large pore size. Polymeric resins such as Amberlite XAD can also be good alternatives for similar separations.

The elution protocol for the HP-20L column was first established with a 500 mL volume (Table 10). After HPLC analysis of the fractions, the target compound was identified in the fraction eluting with 60-70% methanol (G and H).

TABLE 10
Scheme of test runs of the HP-20 column

| ID | Fraction (Elution) |
|---|---|
| C-1702-A | Pre-run |
| C-1702-B | 10% MeOH |
| C-1702-C | 20% MeOH |
| C-1702-D | 30% MeOH |
| C-1702-E | 40% MeOH |
| C-1702-F | 50% MeOH |
| C-1702-G | 60% MeOH |
| C-1702-H | 70% MeOH |
| C-1702-I | 80% MeOH |
| C-1702-J | 90% MeOH |
| C-1702-K | 100% MeOH |
| C-1702-L | 100% Acetonitrile |

This method was scaled up for the larger volumes coming from the fermentation. A glass column (1100 mm length, 100 mm inner diameter) filled with 5 liters of HP-20L was used. The filtered supernatant from fermentation (36 L) was applied to the column. Then the loaded resin was washed and eluted with 0%, 25%, 45%, 65%, 75% and 100% methanol/water solutions, (percent methanol indicated). Samples of each fraction were analyzed by HPLC.

After stepwise elution of the column (Table 11), 22.2 g containing the main amount of Reb A were yielded in 18 L volume (fraction C-1704-E). Ethanol is another solvent that is used to elute molecules from this adsorbent. It is expected that alternate methods could be developed that utilize ethanol or other solvents.

TABLE 11
Scheme of eluting the HP-20 column

| ID | Fraction (Elution) | Amount [g] | Comment |
|---|---|---|---|
| C-1704-A | Pre-run | 855.78 | |
| C-1704-B | Rinse | 45.41 | |
| C-1704-C | 25% MeOH | 51.79 | |
| C-1704-D | 45% MeOH | 55.40 | Reb A (minor) |
| C-1704-E | 65% MeOH | 22.20 | Reb A |
| C-1704-F | 75% MeOH | 1.19 | Reb A (minor) |
| C-1704-G | 100% MeOH | 3.93 | |
| C-1704-H | 100% Acetonitrile | 2.58 | |
| C-1704-I | Concentrated—Pre-run | 0.00 | |
| C-1704-J | Concentrated—Reb A | 22.20 | Reb A |

To remove the water, the entire 18 L volume was applied a second time to the HP-20 column, this time rinsed with 100% water (fraction C-1704-I) and 100% methanol (fraction C-1704-J), which contained the further enriched Reb A fraction, now in 10 L pure methanol (FIG. 4). The solvent was slowly removed by rotary evaporation, not exceeding a temperature of 40° C., until a concentrated solution in methanol (approx. 2 L) was achieved. It is expected that other types of evaporation methods would give equivalent results.

### Example 4

### Enrichment of RebA Using MPLC Fractionation

The concentrated fraction C-1704-J was adsorbed onto a carrier material (Celite 560, particle size>148 μm, SAF) to be further purified by MPLC (medium pressure liquid chromatography) using a gradient of acetonitrile and water.

The MPLC system used was from Kronlab GmbH (Prepcon 4.47 Data System). The stationary phase was Polygoprep 60-50 RP-18 (Macherey-Nagel). This reversed-phase resin has a 60 Å average pore size and particle sizes of 40-63 μm. It is a silica-based resin with an octadecyl phase. It is expected that other C18-based silica resins can also be used for this purification step with the proper solvent and gradients.

The mobile phase consisted of distilled water (A), acetonitrile p.a. (B) and Isopropanol (C). Twenty-two grams were loaded on the column. The gradient employed was as follows:

0-5 minutes, a flow rate of 100 ml/min of 100% A

5.1-10 minutes, flow rate of 130 ml/min of 100% A

10.1-18 minutes, flow rate of 100 ml/min of 80% A: 20% B

18-51 minutes, flow rate of 100 ml/min, gradient from 80% A to 55% A, balance was B. Fractionation started every 2 minutes.

51-61 minutes, % A was decreased linearly from 55 to 10% A (balance B).

At 61-66 minutes the flow rate was increased to 150 ml/min and the system was flushed with 100% B. Fractionation stopped at 66 minutes.

From 61.1 to 70 minutes the system was flushed with 100% C at 30 ml/min.

From 70.1 minutes to 74 minutes the system was washed with 100% C at 75 ml/min.

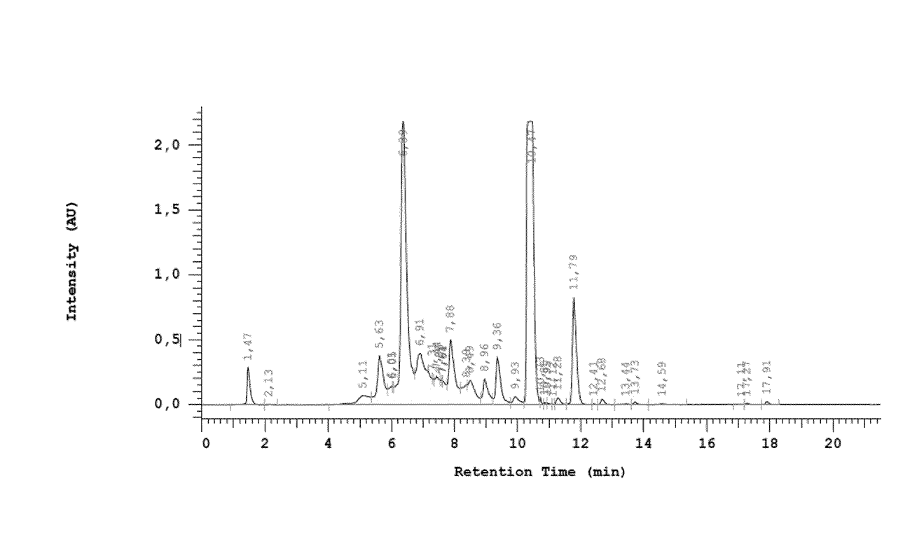

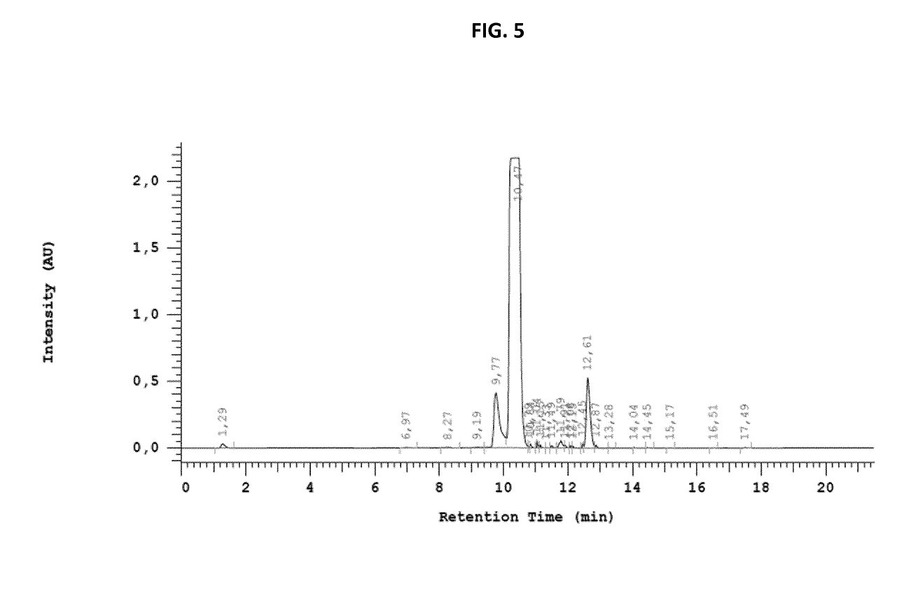

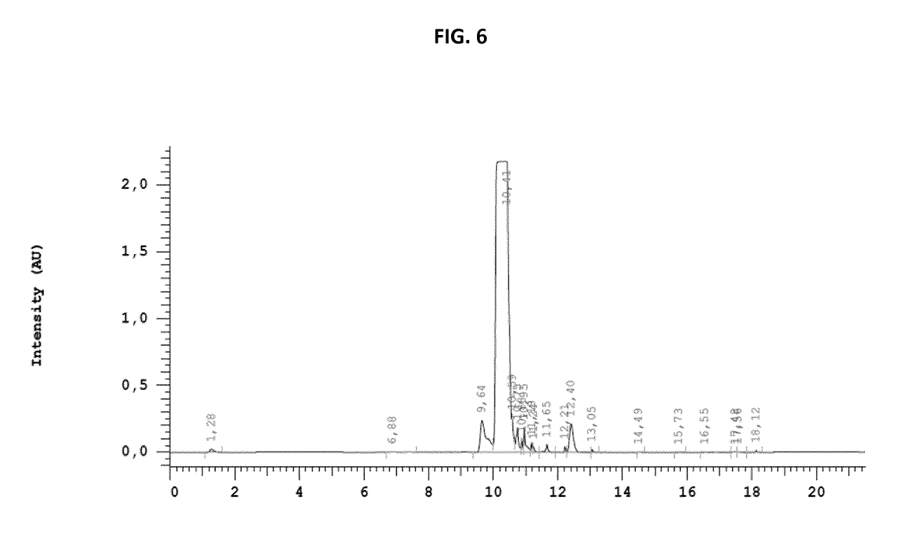

After analyzing the fractions by method 2 (see Example 7), fractions C-1713-11 and C-1713-12 were identified as the main fractions containing Reb A. FIGS. 5 and 6 show the status of the enrichment.

TABLE 12
Yield in grams after HP-20 and MPLC steps

| Sample ID | Description | Amount (g) |
|---|---|---|
| C-1704-J | Post HP-20 enrichment step | 22.2 |
| C-1713-11 | MPLC fraction | 1.44 |
| C-1713-12 | MPLC fraction | 2.51 |
| C-1713-10 | MPLC fraction | 0.61 |
| C-1713-13 | MPLC fraction | 0.53 |
| C-1713-14 | MPLC fraction | 0.08 |

### Example 5

### Crystallization of RebA

Fractions C-1713-11 and -12 were combined for further purification by crystallization (in approximately 40% or higher acetonitrile). Fractions C-1713-10, -13, and -14 also contained Reb A as the major compound, but contained more impurities than 11 and 12. The impurities were not visible in reversed-phase chromatography. The assessment of co-eluting impurities was done by proton-NMR.

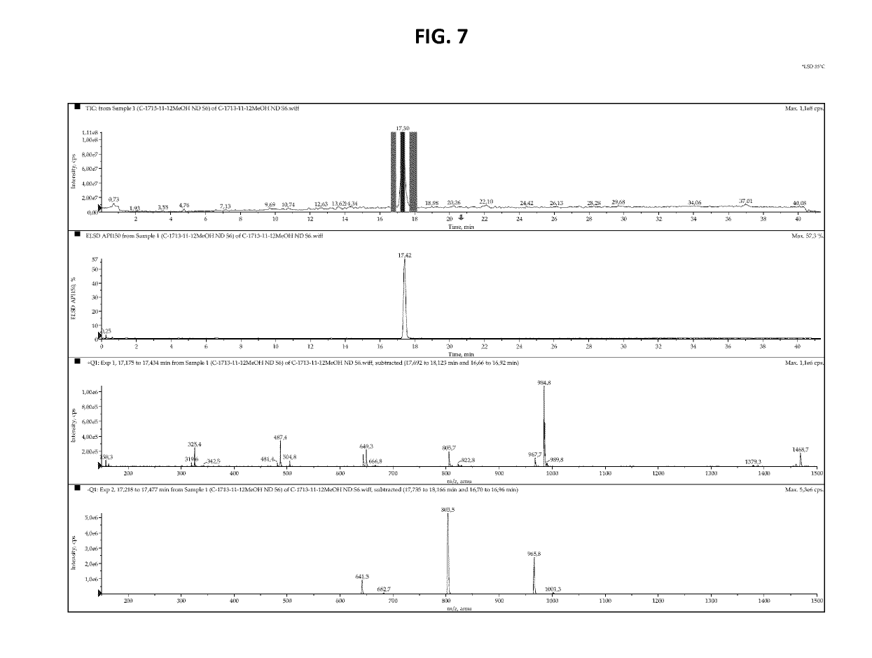

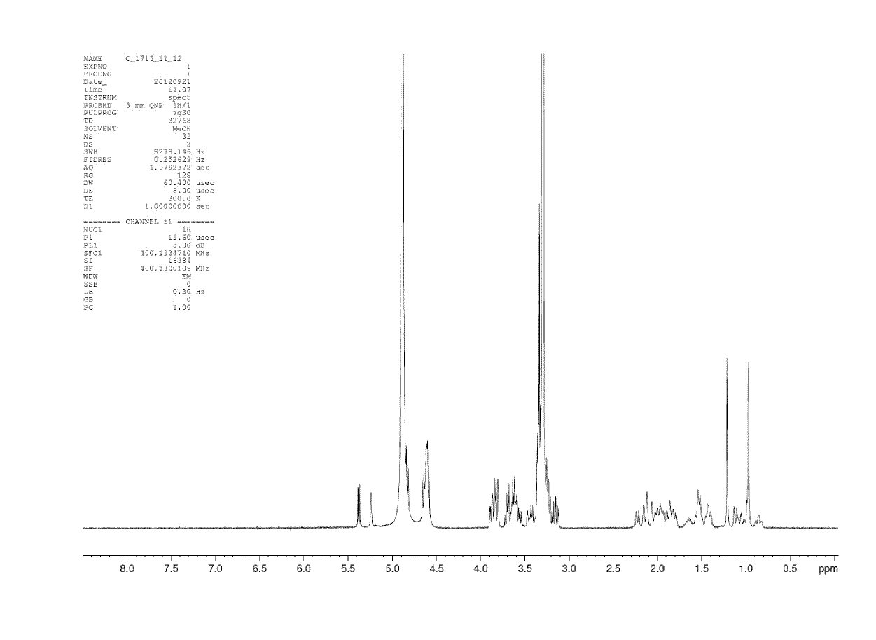

Small amounts of the combined pool of fractions C-1713-11 and -12 were evaporated to remove all acetonitrile, and lyophilized. These samples were used to test the crystallization of Reb A. Solvents used were methanol, acetonitrile, and dioxane. Methanol was chosen as the solvent to use for the crystallization. Briefly, the dried sample was redissolved in approximately 2L of methanol (p.a. grade, 99.9% purity). Evaporation of methanol was done using a Rotavap system until approximately one-half of the methanol remained, followed by resting of the remaining approx. 1,000 ml overnight, which resulted in a white precipitation. 2.16 g of Reb A with a purity of >=95% were obtained. The chromatogram and a ¹H-NMR were used to estimate purity (FIGS. 7 and 8).

### Example 6

### Analytical Methods Used

TABLE 13
Method 1 (HPLC/ELSD/UV/MS); fingerprint

| HPLC System | PE Series 200 | | |
|---|---|---|---|
| MS System | Applied Biosystems API 150, 165 or 365 | | |
| Data System | Analyst 1.3 or Masschrom 1.2.1. | | |
| Stationary | Merck Select B 250 × 4 mm, 5 m | | |
| Phase | | | |
| Flow Rate | 1 ml/min | | |
| Detection | (+/(−)-ESI, Fast-Switching-Mode | | |
| | ELSD (Sedex 75) | | |
| | UV (Merck, 254 nm) | | |
| Sample | 1 to 10 mg/ml in DMSO or methanol | | |
| Concentration | | | |
| Injection | 5 to 30 μl | | |
| Volume | | | |
| Mobile Phase | A: 5 mM ammoniumformiate and 0.1% formic acid | | |
| | B: acetonitrile/methanol = 1:1, 5 mM ammonium | | |
| | formate and 0.1% formic acid (pH 3) | | |
| Gradient | Time [min] | % A | % B |
| | 00.0 | 85 | 15 |
| | 30.0 | 0 | 100 |
| | 35.0 | 0 | 100 |

TABLE 14
Method 2 [Purity Analysis of fractions (HPLC/ELSD)]

| HPLC System | Merck Hitachi | | |
|---|---|---|---|
| Data System | HPLC-Manager D-7000 HSM | | |
| Stationary | Merck Superspher 60 RP-select B 125 × 4 mm, | | |
| Phase | 4 μm | | |
| Flow Rate | 1 ml/min | | |
| Detection | ELSD (Sedex 75); UV(254 nm) | | |
| Injection | 20 μl | | |
| Volume | | | |
| Mobile Phase | A: 5 mM ammonium formate and 0.1% formic acid | | |
| | B: acetonitrile/methanol = 1:1, 5 mM ammonium | | |
| | formate and 0.1% formic acid (pH 3) | | |
| Gradient | time [min] | % A | % B |
| | 0 | 85 | 15 |
| | 15.0 | 0 | 100 |
| | 18.0 | 0 | 100 |

### Example 7

### Sensory Analysis

A sensory evaluation of the sample isolated in Example 5 was conducted by a commercial consumer research and sensory analysis company, using a rapid profiling protocol. The sensory profile of the yeast fermented Reb A product was compared to that of a commercially available RebA product by a panel of 9 tasters (8 trained sensory panellists and 1 Evolve employee), over a two hour period. The commercial product was RebA97, available from PureCircle, Negeri Sembilan, Malaysia, and had a concentration of about 98% w/w RebA. Both products were dissolved in Highland Springs bottled mineral water (Highland Spring Group, Blackford, Pertishire, United Kingdom) at 0.0385% w/v, which is approximately equivalent in sweetness to 8% sucrose.

An appropriate vocabulary was determined using the commercial Reb A solution as a reference sample. As a starting point, the panel used an attribute vocabulary agreed to for previous sweetener sensory analyses, including sweetness onset time, sweetness build, sweetness, bitterness, etc. Panelists discussed and agreed which of the attributes were present in the reference sample and also agreed on approximate intensity scores on a 0-100 scale for each attribute. Table 15 describes each characteristic that was assessed and how each characteristic was scored for the reference sample.

TABLE 15

| | | Score | Score after | Score after | Score after |
|---|---|---|---|---|---|
| Attribute and | | after | 3 d sip | 3 d sip | 3 d sip |
| Attribute Type | Attribute Description | 1st sip | (immediate) | (20 sec) | (2 min) |
| Sweetness onset | Represents how quickly sweetness is first perceived. | 5 | NA | NA | NA |
| (Flavor) | Scored from “no time to sweetness being perceived” | | | | |
| | to “a much delayed time for sweetness.” No time = | | | | |
| | Immediate | | | | |
| Sweetness build | Represents the time to maximum sweetness | 30 | NA | NA | NA |
| (Flavor) | intensity. Scored from no build (i.e. immediately full | | | | |
| | sweetness) to build over very lonq time. | | | | |
| Overall | Overall Intensity of basic taste of sweetness. Scored | NA | 70 | 50 | 50 |
| Sweetness | from no sweetness to very intense sweetness. | | | | |
| (Flavor/Aftertaste) | | | | | |
| Artificial | Intensity of flavour associated with artificial | NA | 55 | 35 | 20 |
| sweetness | sweeteners. The intensity of sweetness of the | | | | |
| (Flavor/Aftertaste) | solution that you would NOT attribute to the basic | | | | |
| | taste found in sucrose. Scored from no perception to | | | | |
| | very intense. | | | | |
| Acidic | Intensity of basic taste of citric acid. Scored from no | NA | 10 | 10 | 10 |
| (Flavor/Aftertaste) | perception to very intense. | | | | |
| Bitter | Intensity of basic taste of caffeine. Scored from no | NA | 10 | 20 | 20 |
| (Flavor/Aftertaste) | perception to very intense. | | | | |
| Liquorice | Intensity of flavor/sensation associated with liquorice | NA | 30 | 35 | 30 |
| (Flavor/Aftertaste) | sweets. Scored from no perception to very intense. | | | | |
| Body/thickness | A measure of how thick or syrupy the solution is. | NA | 40 | NA | NA |
| (Mouthfeel) | Scored from no body to very thick body. (Not = No | | | | |
| | body). Assessed with tongue and soft tissue of | | | | |
| | mouth | | | | |
| Tingling | The degree to which the solution produces the | NA | NA | 10 | 5 |
| (Mouthfeel) | tingling sensation of the tongue and soft tissue of | | | | |
| | mouth. Assessed with tongue and soft tissue of | | | | |
| | mouth | | | | |
| Mouth drying | The degree to which the solution produces a drying | NA | NA | 35 | 35 |
| (Mouthfeel) | sensation in the mouth. Scored from not drying to | | | | |
| | very drying. Assessed with tongue and soft tissue of | | | | |
| | mouth. | | | | |
| Mouth coating | The degree to which the solution coats or adheres to | NA | NA | 30 | 30 |
| (Mouthfeel) | surfaces of the mouth. Scored from not mouth | | | | |
| | coating to very mouth coating. Assessed with tongue | | | | |
| | and soft tissue of mouth. | | | | |
| Mouth watering | The degree to which the solution evokes saliva | NA | NA | 0 | 20 |
| (Mouthfeel) | production in the mouth. Scored from not mouth | | | | |
| | watering to very mouth watering. Assessed with | | | | |
| | tongue and soft tissue of mouth. | | | | |

After a break, the panelists were presented one blinded sample of the yeast-derived RebA solution and one blinded sample of the commercial RebA solution. Sample were presented in a balanced order to the panelists. For each blinded sample, panelists carried out the following tasting procedure. Panelists were instructed take one sip of the sample and score the Sweetness Build and Sweetness Onset attributes. A second and third sip of the sample were then taken, and panelists were instructed to score Flavor Attributes and Mouth Feel attribute Body/thickness. Each panelist then took a final sip of the sample and was asked to score Aftertaste and Mouth Sensations at 20 seconds and 120 seconds after the final sip.

All panellist scores were entered into a computer using Compusense® data collection software (Compusense, Guelph, Ontario, Canada). Agreed reference scores were marked on the scales as a guide. A rest period of 5 minutes duration was observed between samples and palate cleansers of Carr's® water crackers (Kellogg's, Battle Creek, Mich.) and Highland Spring bottled mineral water applied during this time.

After one replicate of testing had been completed, panelists discussed differences between products. Panelists thought there were some subtle differences between products but no new flavors, off flavors, or mouth sensations were experienced. It was therefore decided to carry out a second replicate testing of the two Reb A samples using the same vocabulary and different blinding codes. The same tasting procedure was used for the second replicate as that used for the first replicate.

Mean scores were calculated for each attribute and sample over the two replicates (Rep 1 and Rep 2). Table 16 provides the mean scores for each attribute. Scores in bold shown a statistically significant difference between the two RebA samples.

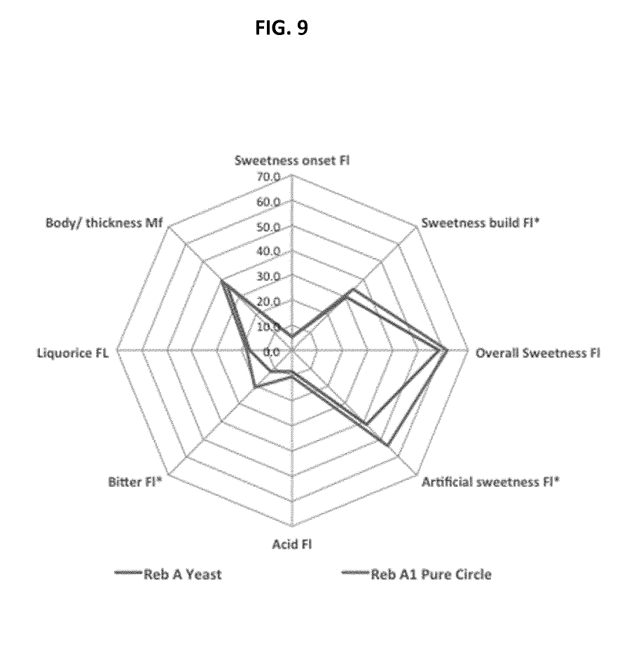

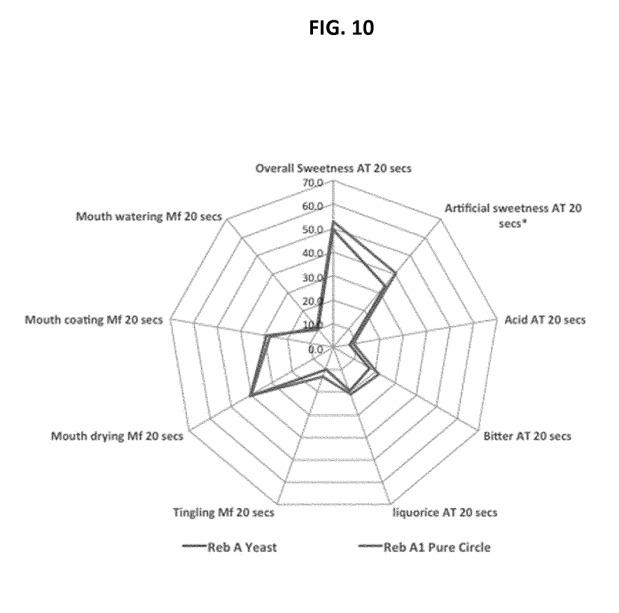

Spider plots (FIGS. 9 and 10) were drawn to compare the profiles of the two samples.

Based on sensory evaluation by the rapid profiling protocol described above, both RebA samples had a relatively immediate sweetness onset. However, the commercial RebA sample had a sweetness build score that was significantly longer than that of the yeast derived RebA sample, i.e., the commercial product took significantly longer to peak in sweetness intensity than Reb A from yeast. The yeast-derived Reb A sample had an artificial sweetness score at 20 seconds that was significantly less than those of the commercial RebA sample. The yeast-derived RebA sample also had a bitter score that was lower at 20 seconds that that of the commercial RebA sample, although not statistically significantly different. Both samples had a slight liquorice flavour and there was no statistically significant difference in liquorice flavour intensity. There was no significant difference for overall sweetness intensity or mouthfeel/body. There was no significant difference in attribute scores at 120 seconds. See FIG. 10.

There was no significant difference between the two samples in mouthfeel/mouth sensations, both samples being described by panellists as somewhat mouthdrying and mouthcoating. There were no reports of off flavors present in the yeast derived RebA product.

TABLE 16

| | Yeast derived | Commercial | | |
|---|---|---|---|---|
| Attribute | Reb A | RebA | LSD | Prob |
| Sweetness onset Fl. | 5.5 | 5.6 | 3.8 | 0.9485 |
| Sweetness build Fl. | 30.2 | 34.5 | 3.9 | 0.0345 |
| Overall Sweetness Fl. | 58.5 | 61.7 | 8.0 | 0.3840 |
| Artificial sweetness Fl. | 41.8 | 54.0 | 8.8 | 0.0127 |
| Acid Fl. | 8.3 | 10.6 | 6.2 | 0.4189 |
| Bitter Fl. | 11.8 | 20.7 | 8.6 | 0.0448 |
| Liquorice FL | 17.2 | 18.0 | 9.9 | 0.8562 |
| Body/ thickness Mf. | 37.1 | 39.4 | 4.1 | 0.2330 |
| Overall Sweetness At. | 49.6 | 52.8 | 5.5 | 0.2096 |
| Artificial sweetness At. | 33.8 | 40.8 | 5.8 | 0.0240 |
| Acid At. | 6.9 | 8.5 | 3.8 | 0.3617 |
| Bitter At. | 17.2 | 22.1 | 7.6 | 0.1707 |
| liquorice At. | 19.2 | 21.1 | 10.1 | 0.6776 |
| Tingling Mf. | 9.8 | 13.0 | 4.4 | 0.1212 |
| Mouth drying Mf. | 40.8 | 40.1 | 5.8 | 0.8144 |
| Mouth coating Mf. | 28.9 | 27.4 | 6.8 | 0.6260 |
| Mouth watering Mf. | 10.1 | 10.9 | 6.4 | 0.7848 |
| Overall Sweetness At. | 46.2 | 49.6 | 5.1 | 0.1639 |
| Artificial sweetness At. | 30.0 | 32.6 | 3.3 | 0.1036 |
| Acid At. | 5.5 | 8.9 | 2.9 | 0.0269 |
| Bitter At. | 18.1 | 20.6 | 5.3 | 0.3150 |
| Liquorice At. | 16.2 | 16.3 | 6.6 | 0.9850 |
| Tingling Mf. | 8.8 | 12.9 | 5.4 | 0.1178 |
| Mouth drying Mf. | 39.8 | 42.7 | 6.9 | 0.3643 |
| Mouth coating Mf. | 24.7 | 26.3 | 3.8 | 0.3771 |
| Mouth watering Mf. | 15.7 | 14.7 | 6.0 | 0.7021 |

“Fl” refers to “flavor.”
“Mf” refers to “mouthfeel.”
“At” refers to “aftertaste.”
“LSD” refers to “least significant difference.”
“Prob” refers to the likelihood of a statically significant difference at the 95% confidence level.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A composition comprising at least 90% w/w rebaudioside A, said composition having one or more of the following:
a) a statistically significant decrease in a sweetness build score relative to a Stevia-derived rebaudioside A composition;
b) a statistically significant decrease in an artificial sweetness score relative to a Stevia-derived rebaudioside A composition;
c) a statistically significant decrease in a bitterness score relative to a Stevia-derived rebaudioside A composition; and
d) a statistically significant decrease in two-minute acid score relative to a Stevia-derived rebaudioside A composition, said scores determined in a standardized sensory panel evaluation.

2. The composition of claim 1, wherein said rebaudioside A is produced in a recombinant microorganism.

3. The composition of claim 2, wherein said recombinant microorganism is a yeast.

4. The composition of claim 3, wherein said yeast is Saccharomyces cerevisiae.

5. A food product comprising the composition of claim 1.

6. A method for producing a steviol glycoside product, comprising:
a) fermenting a recombinant microorganism capable of producing at least 1 g/L of said steviol glycoside in a culture medium or carrying out biocatalysis in a reaction mixture with one or more of the enzymes listed in Sections I-A, I-B, I-C, or I-D of the specification, to produce the steviol glycoside; and
b) purifying said steviol glycoside from said culture medium or from said reaction mixture, using one or more purification steps selected from the group consisting of:
(i) fractionation on an adsorbent resin;
(ii) fractionation on a reversed phase resin;
(iii) crystallization; and
(iv) a drying step,
thereby producing said steviol glycoside product,
said steviol glycoside product having a statistically significant difference in at least one sensory attribute relative to a Stevia-derived steviol glycoside product, said sensory attribute evaluated in a standardized sensory panel evaluation.

7. The method of claim 6, wherein said steviol glycoside product comprises at least 90% w/w rebaudioside A.

8. The method of claim 6, wherein said steviol glycoside product comprises at least 95% w/w rebaudioside A.

9. The method of claim 6, wherein said steviol glycoside product comprises at least 98% w/w rebaudioside A.

10. The method of any one of claims 6 to 9, wherein said microorganism is Saccharomyces cerevisiae.

11. The method of any one of claims 6 to 10, said method comprising a crystallization step selected from the group consisting of anti-solvent crystallization, temperature-based crystallization, and evaporative crystallization.

12. The method of any one of claims 6 to 11, said method comprising fractionation on a synthetic polyaromatic gel.

13. The method of any one of claims 6 to 12, said method comprising fractionation on said reversed phase resin using medium pressure liquid chromatography.
